(19) United States
(12) Patent Application Publication
(10) Pub. No.: US 2024/0261414 A1
(43) Pub. Date: Aug. 8, 2024

(54) FATTY ACID AMIDE HYDROLASE (FAAH) CLEAVABLE PRODRUGS OF BRAIN TARGETING ACTIVES AND COMBINATION WITH PERIPHERALLY RESTRICTED FAAH INHIBITORS

(71) Applicant: Autobahn Therapeutics, Inc., San Diego, CA (US)

(72) Inventors: Brian Andrew STEARNS, San Diego, CA (US); Jill Melissa BACCEI, San Diego, CA (US); Jason Randall HARRIS, San Diego, CA (US)

(21) Appl. No.: 18/558,709

(22) PCT Filed: May 6, 2022
(86) PCT No.: PCT/US2022/028164

§ 371 Date: Nov. 2, 2023

Related U.S. Application Data
(60) Provisional application No. 63/185,253, filed on May 6, 2021.

(51) Int. Cl. A61K 47/54; A61K 31/4545

(52) U.S. Cl. CPC A61K 47/542; A61K 31/4545; A61K 47/545

(57) ABSTRACT

Provided herein are fatty acid amide (FAAH) cleavable prodrugs of compounds that modulate a target in the brain including sphingosine-1-phosphate receptor (S1P1), lysophosphatidic acid receptor 1 (LPA1), G-protein coupled receptor 120 (GPR120), prostacyclin (PGI2), and transthyretin (TTR). Pharmaceutical compositions comprising these prodrugs, including in combination with a peripherally restricted FAAH inhibitor, and at least one pharmaceutically acceptable excipient, are also provided, and the use of these compounds and compositions in the treatment of CNS diseases or disorders.

### CROSS-REFERENCE

This application claims the benefit of U.S. Provisional Application No. 63/185,253 filed on May 6, 2021, which is incorporated herein by reference in its entirety.

### BACKGROUND OF THE INVENTION

The blood-brain barrier is composed of tightly linked endothelial cells that limit the passage of pathogens and specific types of small and large molecules from the blood into the brain. This critical protective function also restricts the diffusion of therapeutics into the brain representing a major challenge to the development of new medicines for CNS diseases.

### SUMMARY OF THE INVENTION

In one aspect provided herein is a pharmaceutical composition comprising a fatty acid amide hydrolase (FAAH) cleavable prodrug of Formula (I), or a pharmaceutically acceptable salt or solvate thereof:

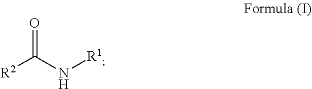

wherein:
R¹ is an amide prodrug moiety, wherein the prodrug of Formula (I) is enzymatically cleaved by fatty acid amide hydrolase (FAAH) in the brain to release

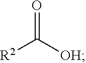

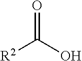

is a moiety that modulates a target in the brain; and a pharmaceutically acceptable excipient; further comprising a peripherally restricted FAAH inhibitor.

In some embodiments, the target is selected from sphingosine-1-phosphate receptor 1 (S1P1), lysophosphatidic acid receptor 1 (LPA1), G-protein coupled receptor 120 (GPR120), prostacyclin (PGI2), and transthyretin (TTR).

In some embodiments, the target is S1P1. In some embodiments, the target is S1P1, wherein R² is selected from

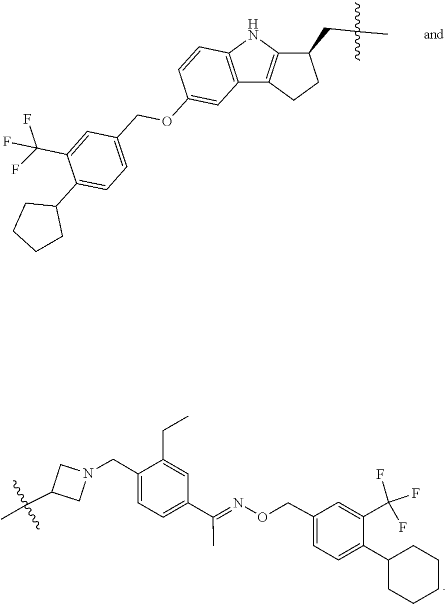 [SMILES: *C[C@H]1CCc2c1[nH]c1ccc(OCc3ccc(C4CCCC4)c(C(F)(F)F)c3)cc21 ; *C1CN(Cc2ccc(/C(C)=N/OCc3ccc(C4CCCCC4)c(C(F)(F)F)c3)cc2CC)C1]

In some embodiments, the target is LPA1. In some embodiments, the target is LPA1, wherein R² is selected from

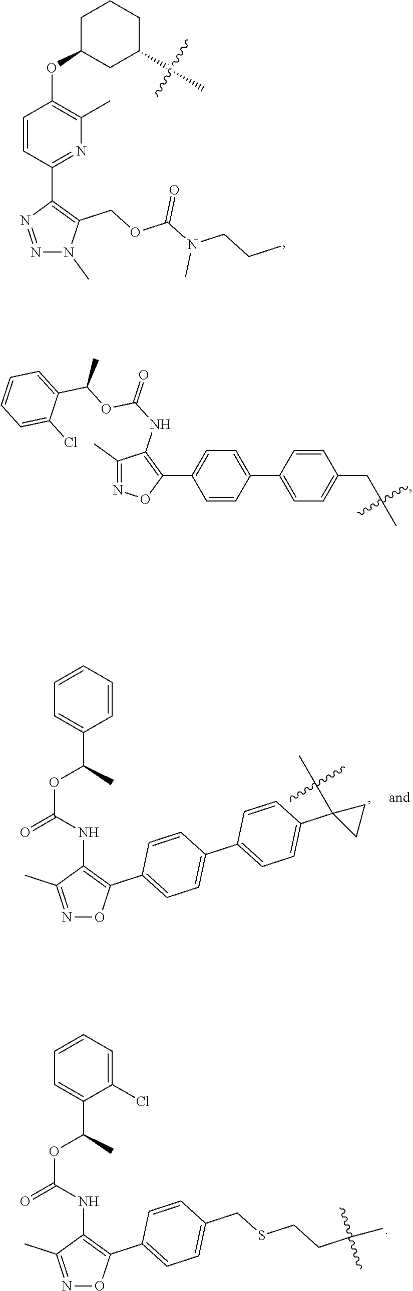

In some embodiments, the target is GPR120. In some embodiments, the target is GPR120, wherein R² is

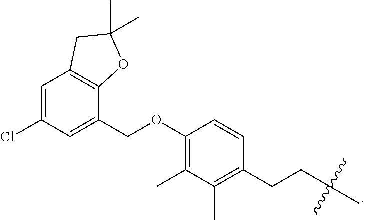 [SMILES: *CCc1ccc(OCc2cc(Cl)cc3c2OC(C)(C)C3)c(C)c1C]

In some embodiments, the target is TTR. In some embodiments, the target is TTR, wherein R² is selected from

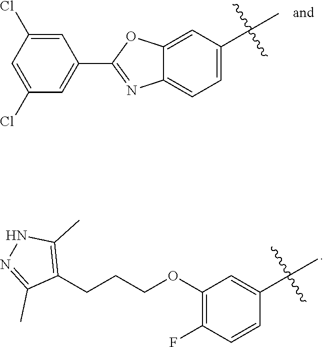 [SMILES: *c1ccc2nc(-c3cc(Cl)cc(Cl)c3)oc2c1 ; *c1ccc(F)c(OCCCc2c(C)n[nH]c2C)c1]

In some embodiments, the target is PGI2. In some embodiments, the target is PGI2, wherein R² is

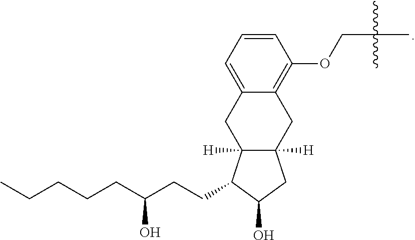 [SMILES: *COc1cccc2c1C[C@H]1C[C@@H](O)[C@H](CC[C@@H](O)CCCCC)[C@H]1C2]

In some embodiments, R¹ is selected from optionally substituted C₁₋₆alkyl, optionally substituted C₃₋₆cycloalkyl, optionally substituted C₂₋₉heterocycloalkyl, optionally substituted C₆₋₁₀aryl, and optionally substituted C₁₋₉heteroaryl. In some embodiments, R¹ is selected from optionally substituted C₁₋₆alkyl, optionally substituted C₃₋₆cycloalkyl, optionally substituted C₂₋₉heterocycloalkyl, optionally substituted C₆₋₁₀aryl, and optionally substituted C₁₋₉heteroaryl, wherein C₁₋₆alkyl, C₃₋₆cycloalkyl, C₂₋₉heterocycloalkyl, C₆₋₁₀aryl, C₁₋₉heteroaryl are optionally substituted with 1, 2, 3, 4, or 5 groups independently selected from halogen, —CN, —OH, —NH₂, —N(H)(C₁₋₆alkyl), N(C₁₋₆alkyl)₂, —C(O)OH, —C(O)O—C₁₋₆alkyl, —C(O)NH₂, —C(O)N(H)(C₁₋₆alkyl), —C(O)N(C₁₋₆alkyl)₂, —C(O)C₁₋₆alkyl, —S(O)₂C₁₋₆alkyl, —S(O)₂NH₂, —S(O)₂N(H)(C₁₋₆alkyl), —S(O)₂N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments, R¹ is selected from unsubstituted C₁₋₆alkyl, unsubstituted C₃₋₆cycloalkyl, unsubstituted C₂₋₉heterocycloalkyl, unsubstituted C₆₋₁₀aryl, and unsubstituted C₁₋₉heteroaryl. In some embodiments, R¹ is selected from

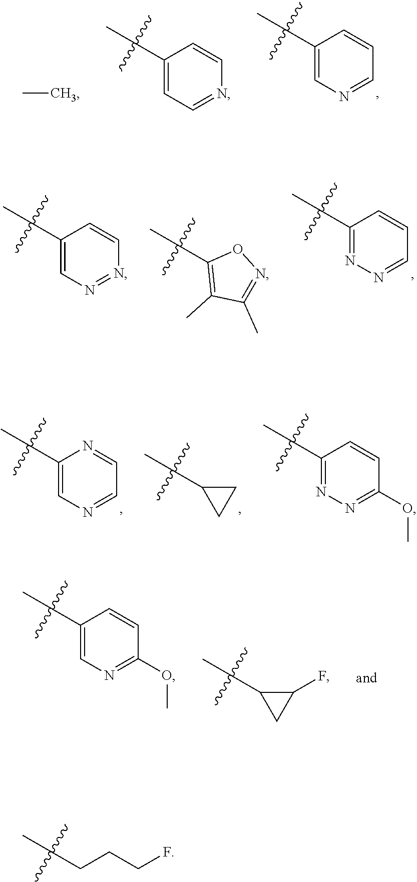

In some embodiments, the peripherally restricted FAAH inhibitor is ASP-3652.

In another aspect is a method of treating a CNS disease or disorder in a patient in need thereof comprising administering to the patient a therapeutically effective amount of a pharmaceutical composition described herein. In some embodiments, the CNS disease or disorder is selected from multiple sclerosis, amyotrophic lateral sclerosis, Huntington's disease, Parkinson's disease, and Alzheimer's disease. In some embodiments, the CNS disease or disorder is selected from epilepsy, ischemic stroke, traumatic brain injury, and autoimmune encephalomyelitis. In some embodiments, the CNS disease or disorder is selected from ischemic stroke, schizophrenia, depression, mood disorders, attention deficit hyperactivity disorder, post-traumatic stress disorder, and Alzheimer-type dementia. In some embodiments, the CNS disease or disorder is selected from familial amyloidotic polyneuropathy, familial leptomeningeal amyloidosis, Alzheimer's disease, stroke, dementia, transitory focal neurological episodes, cognitive dysfunction, and CNS amyloidosis. In some embodiments, the CNS disease or disorder is selected from Degos disease, reversible cerebral vasoconstriction syndrome, Sneddon's syndrome, amyloid-beta-related angiopathy, Susac syndrome, and neurosarcoidosis.

In another aspect is a method of increasing the concentration of

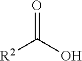

in the brain of a patient comprising administering to the patient a pharmaceutical composition described herein.

In another aspect described herein is a compound, or a pharmaceutically acceptable salt or solvate thereof, of Formula (II):

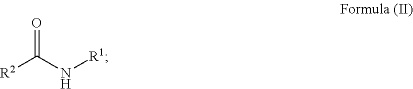

wherein:
R¹ is an amide prodrug moiety, wherein the amide prodrug moiety is enzymatically cleaved by fatty acid amide hydrolase (FAAH) in the brain to release

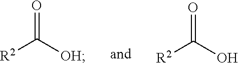

is a moiety that modulates S1P1 in the brain.

In some embodiments is a compound of Formula (II), wherein R² is selected from

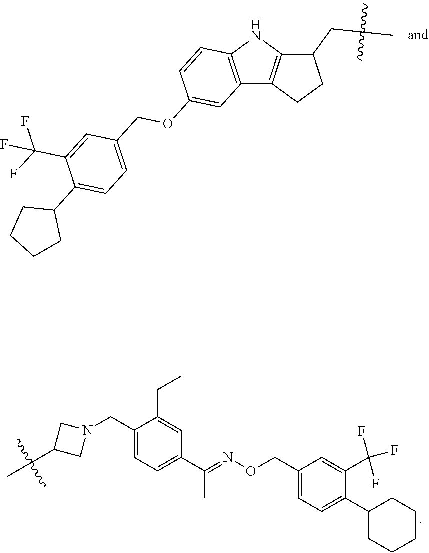 [SMILES: *CC1CCc2c1[nH]c1ccc(OCc3ccc(C4CCCC4)c(C(F)(F)F)c3)cc21 ; *C1CN(Cc2ccc(/C(C)=N/OCc3ccc(C4CCCCC4)c(C(F)(F)F)c3)cc2CC)C1]

In another aspect described herein is a compound, or a pharmaceutically acceptable salt or solvate thereof, of Formula (III):

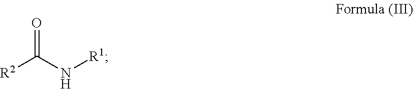

wherein:
R¹ is an amide prodrug moiety, wherein the amide prodrug moiety is enzymatically cleaved by fatty acid amide hydrolase (FAAH) in the brain to release

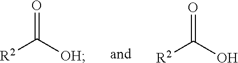

is a moiety that modulates LPA1 in the brain.

In some embodiments is a compound of Formula (III), wherein R² is selected from

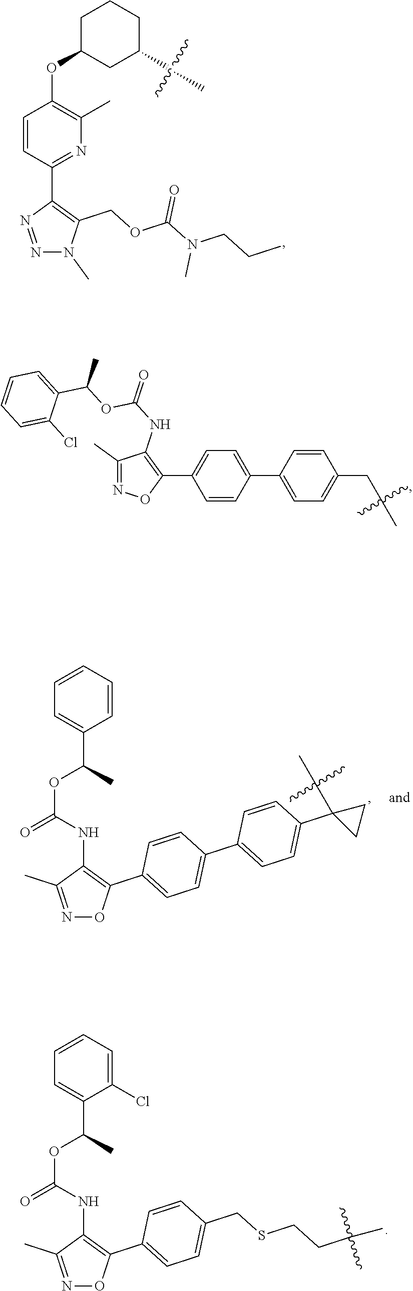

In another aspect described herein is a compound, or a pharmaceutically acceptable salt or solvate thereof, of Formula (IV):

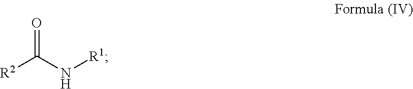

wherein:
R¹ is an amide prodrug moiety, wherein the amide prodrug moiety is enzymatically cleaved by fatty acid amide hydrolase (FAAH) in the brain to release

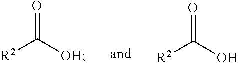

is a moiety that modulates GPR120 in the brain.

In some embodiments is a compound of Formula (IV), wherein R² is

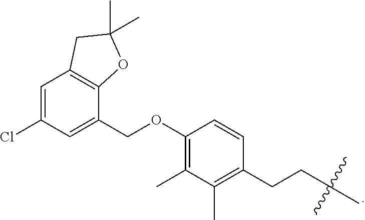 [SMILES: *CCc1ccc(OCc2cc(Cl)cc3c2OC(C)(C)C3)c(C)c1C]

In some embodiments is a compound of Formula (II), (III), or (IV), wherein R¹ is selected from optionally substituted C₁₋₆alkyl, optionally substituted C₃₋₆cycloalkyl, optionally substituted C₂₋₉heterocycloalkyl, optionally substituted C₆₋₁₀aryl, and optionally substituted C₁₋₉ heteroaryl. In some embodiments is a compound of Formula (II), (III), or (IV), wherein R¹ is selected from C₁₋₆alkyl, C₃₋₆cycloalkyl, C₂₋₉heterocycloalkyl, C₆₋₁₀aryl, and C₁₋₉heteroaryl, wherein C₁₋₆alkyl, C₃₋₆cycloalkyl, C₂₋₉heterocycloalkyl, C₆₋₁₀aryl, and C₁₋₉heteroaryl are optionally substituted with 1, 2, 3, 4, or 5 groups independently selected from halogen, —CN, —OH, —NH₂, —N(H)(C₁₋₆alkyl), N(C₁₋₆alkyl)₂, —C(O)OH, —C(O)O—C₁₋₆alkyl, —C(O)NH₂, —C(O)N(H)(C₁₋₆alkyl), —C(O)N(C₁₋₆alkyl)₂, —C(O)C₁₋₆alkyl, —S(O)₂C₁₋₆alkyl, —S(O)₂NH₂, —S(O)₂N(H)(C₁₋₆alkyl), —S(O)₂N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments is a compound of Formula (II), (III), or (IV), wherein R¹ is selected from unsubstituted C₁₋₆alkyl, unsubstituted C₃₋₆ cycloalkyl, unsubstituted C₂₋₉heterocycloalkyl, unsubstituted C₆₋₁₀aryl, and unsubstituted C₁₋₉heteroaryl.

In another aspect described herein is a compound, or a pharmaceutically acceptable salt or solvate thereof, of Formula (V):

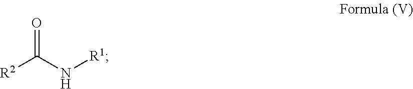

wherein:
R¹ is an amide prodrug moiety, wherein the amide prodrug moiety is enzymatically cleaved by fatty acid amide hydrolase (FAAH) in the brain to release

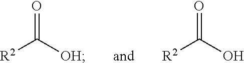

is a moiety that modulates TTR in the brain.

In some embodiments is a compound of Formula (V), wherein R² is

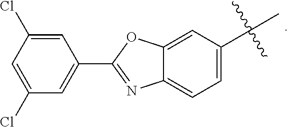 [SMILES: *c1ccc2nc(-c3cc(Cl)cc(Cl)c3)oc2c1]

In some embodiments is a compound of Formula (V), wherein R¹ is selected from C₁₋₄alkyl, C₁₋₄haloalkyl, C₃₋₆cycloalkyl, C₆₋₁₀aryl, and C₁₋₉heteroaryl, wherein C₃₋₆cycloalkyl, C₆₋₁₀aryl, and C₁₋₉heteroaryl are optionally substituted with 1, 2, 3, 4, or 5 groups independently selected from halogen, —CN, —OH, —NH₂, —N(H)(C₁₋₆alkyl), N(C₁₋₆alkyl)₂, —C(O)OH, —C(O)O—C₁₋₆alkyl, —C(O)NH₂, —C(O)N(H)(C₁₋₆alkyl), —C(O)N(C₁₋₆alkyl)₂, —C(O)C₁₋₆alkyl, —S(O)₂C₁₋₆alkyl, —S(O)₂NH₂, —S(O)₂N(H)(C₁₋₆alkyl), —S(O)₂N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments is a compound of Formula (V), wherein R¹ is selected from unsubstituted C₁₋₄alkyl, unsubstituted C₃₋₆cycloalkyl, unsubstituted C₆₋₁₀aryl, and unsubstituted C₁₋₉heteroaryl.

In some embodiments is a compound of Formula (V), wherein R² is

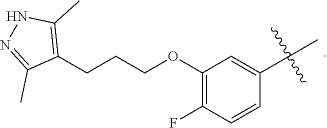 [SMILES: *c1ccc(F)c(OCCCc2c(C)n[nH]c2C)c1]

In some embodiments is a compound of Formula (V), wherein R¹ is selected from —CH₃, C₁₋₆haloalkyl, C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with 1, 2, 3, 4, or 5 groups independently selected from halogen, —CN, —OH, —NH₂, —N(H)(C₁₋₆alkyl), N(C₁₋₆alkyl)₂, —C(O)OH, —C(O)O—C₁₋₆alkyl, —C(O)NH₂, —C(O)N(H)(C₁₋₆alkyl), —C(O)N(C₁₋₆alkyl)₂, —C(O)C₁₋₆alkyl, —S(O)₂C₁₋₆alkyl, —S(O)₂NH₂, —S(O)₂N(H)(C₁₋₆alkyl), —S(O)₂N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments is a compound of Formula (V), wherein R¹ is selected from —CH₃, unsubstituted C₃₋₆cycloalkyl, unsubstituted C₂₋₉heterocycloalkyl, unsubstituted C₆₋₁₀aryl, and unsubstituted C₁₋₉heteroaryl.

In another aspect described herein is a compound, or a pharmaceutically acceptable salt or solvate thereof, of Formula (VI):

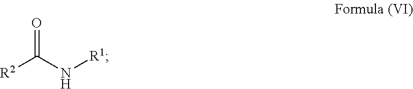

wherein:
R¹ is an amide prodrug moiety, wherein the amide prodrug moiety is enzymatically cleaved by fatty acid amide hydrolase (FAAH) in the brain to release

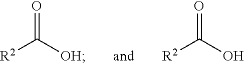

is a moiety that modulates PGI2 in the brain.

In some embodiments is a compound of Formula (VI), wherein R² is

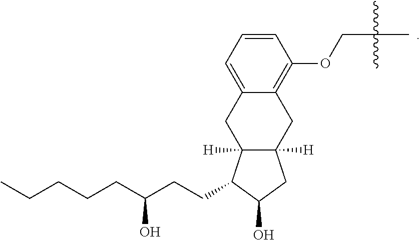 [SMILES: *COc1cccc2c1C[C@H]1C[C@@H](O)[C@H](CC[C@@H](O)CCCCC)[C@H]1C2]

In some embodiments is a compound of Formula (VI), wherein R¹ is selected from —CH₃, optionally substituted C₃₋₆cycloalkyl, optionally substituted C₂₋₉heterocycloalkyl, optionally substituted C₆₋₁₀aryl, and optionally substituted C₂₋₉heteroaryl, wherein C₃₋₆cycloalkyl, C₂₋₉heterocycloalkyl, C₆₋₁₀aryl, C₁₋₉heteroaryl are optionally substituted with 1, 2, 3, 4, or 5 groups independently selected from halogen, —CN, —OH, —NH₂, —N(H)(C₁₋₆alkyl), N(C₁₋₆alkyl)₂, —C(O)OH, —C(O)O—C₁₋₆alkyl, —C(O)NH₂, —C(O)N(H)(C₁₋₆alkyl), —C(O)N(C₁₋₆alkyl)₂, —C(O)C₁₋₆alkyl, —S(O)₂C₁₋₆alkyl, —S(O)₂NH₂, —S(O)₂N(H)(C₁₋₆alkyl), —S(O)₂N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments is a compound of Formula (VI), wherein R¹ is selected from —CH₃, unsubstituted C₃₋₆cycloalkyl, unsubstituted C₂₋₉heterocycloalkyl, unsubstituted C₆₋₁₀aryl, and unsubstituted C₁₋₉heteroaryl.

In some embodiments is a compound of Formula (II), (III), (IV), (V), or (VI), wherein R¹ is selected from

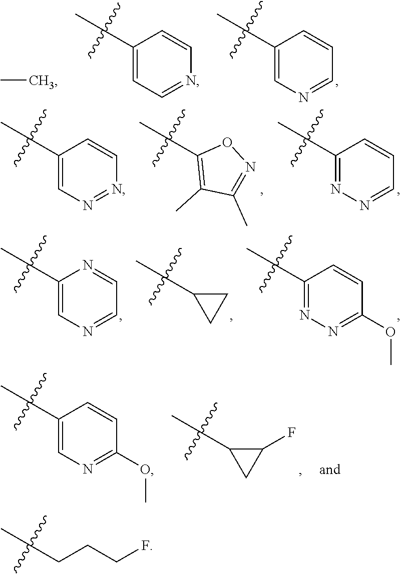

In another aspect is a pharmaceutical composition comprising a compound of Formula (II), (III), (IV), (V), or (VI), or a pharmaceutically acceptable salt or solvate thereof, and a pharmaceutically acceptable excipient. In some embodiments, the pharmaceutical composition comprises a peripherally restricted FAAH inhibitor. In some embodiments, the pharmaceutical composition comprises a peripherally restricted FAAH inhibitor, wherein the peripherally restricted FAAH inhibitor is ASP-3652.

In another aspect is a method of treating a CNS disease or disorder in a patient in need thereof comprising administering to the patient a compound of Formula (II), (III), (IV), (V), or (VI), or a pharmaceutically acceptable salt or solvate thereof. In another aspect is a method of treating a CNS disease or disorder in a patient in need thereof comprising administering to the patient a pharmaceutical composition comprising a compound of Formula (II), (III), (IV), (V), or (VI), or a pharmaceutically acceptable salt or solvate thereof, and a pharmaceutically acceptable excipient. In some embodiments, the CNS disease or disorder is selected from multiple sclerosis, amyotrophic lateral sclerosis, Huntington's disease, Parkinson's disease, and Alzheimer's disease. In some embodiments, the CNS disease or disorder is selected from epilepsy, ischemic stroke, traumatic brain injury, and autoimmune encephalomyelitis. In some embodiments, the CNS disease or disorder is selected from ischemic stroke, schizophrenia, depression, mood disorders, attention deficit hyperactivity disorder, post-traumatic stress disorder, and Alzheimer-type dementia. In some embodiments, the CNS disease or disorder is selected from familial amyloidotic polyneuropathy, familial leptomeningeal amyloidosis, Alzheimer's disease, stroke, dementia, transitory focal neurological episodes, cognitive dysfunction, and CNS amyloidosis. In some embodiments, the CNS disease or disorder is selected from Degos disease, reversible cerebral vasoconstriction syndrome, Sneddon's syndrome, amyloid-beta-related angiopathy, Susac syndrome, and neurosarcoidosis.

In another aspect is a method of increasing the concentration of

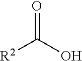

in the brain of a patient comprising administering to the patient a compound described herein.

### DETAILED DESCRIPTION OF THE INVENTION

Fatty acid amide hydrolase (FAAH) is an integral membrane serine hydrolase that degrades the fatty acid amide family of signaling lipids and can hydrolyze select amide prodrugs. FAAH is highly conserved between species and is expressed in many tissues, including the central nervous system (CNS), to varying degrees. Select carboxylic acids can be converted to more permeable amide prodrugs which are then capable of passing through the blood brain barrier where they can be converted to active molecules through the action of FAAH upon the prodrug. This results in the delivery of higher amounts of the carboxylic acid to the CNS as compared to dosing the parent alone. However, peripherally expressed FAAH simultaneously hydrolyzes the prodrug resulting in a considerable amount of non-productive prodrug conversion. Co-administration of a peripherally restricted FAAH inhibitor with a CNS permeable FAAH convertible prodrug increases the selectivity of prodrug delivery to the CNS. It also results in lower exposures of the parent molecule in plasma and peripheral tissue than what is observed when dosing the prodrug alone.

### Certain Terminology

The singular forms “a,” “an,” and “the” include plural referents unless the context clearly dictates otherwise. Thus, for example, reference to “a drug” includes reference to one or more of such drugs, and reference to “an excipient” includes reference to one or more of such excipients. When ranges are used herein, all combinations and sub-combinations of ranges and specific embodiments therein are intended to be included. The term “about” when referring to a number or a numerical range means that the number or numerical range referred to is an approximation within experimental variability (or within statistical experimental error), and thus the number or numerical range varies between 1% and 15% of the stated number or numerical range.

The terms “formulation” and “composition,” as used herein, are used interchangeably and refer to a mixture of two or more compounds, elements, or molecules. In some aspects the terms “formulation” and “composition” may be used to refer to a mixture of one or more active agents with a carrier or other excipients.

The terms “active agent,” “active pharmaceutical agent,” “drug,” “active ingredient,” and variants thereof are used interchangeably to refer to an agent or substance that has measurable specified or selected physiologic activity when administered to a subject in a significant or effective amount.

“Pharmaceutically acceptable salt” includes both acid and base addition salts. A pharmaceutically acceptable salt of any one of the compounds described herein is intended to encompass any and all pharmaceutically suitable salt forms. Preferred pharmaceutically acceptable salts of the compounds described herein are pharmaceutically acceptable acid addition salts, and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are prepared by contacting the free base forms with a sufficient amount of the desired acid to produce the salt.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. In some embodiments, pharmaceutically acceptable base addition salts are formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts, and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins, and the like. See Berge et al., supra.

It should be understood that a reference to a pharmaceutically acceptable salt includes the solvent addition forms (solvates). Solvates contain either stoichiometric or non-stoichiometric amounts of a solvent, and are formed during the process of product formation or isolation with pharmaceutically acceptable solvents such as water, ethanol, methanol, methyl tert-butyl ether (MTBE), diisopropyl ether (DIPE), ethyl acetate, isopropyl acetate, isopropyl alcohol, methyl isobutyl ketone (MIBK), methyl ethyl ketone (MEK), acetone, nitromethane, tetrahydrofuran (THF), dichloromethane (DCM), dioxane, heptanes, toluene, anisole, acetonitrile, and the like. In one aspect, solvates are formed using, but not limited to, Class 3 solvent(s). Categories of solvents are defined in, for example, the International Conference on Harmonization of Technical Requirements for Registration of Pharmaceuticals for Human Use (ICH), “Impurities: Guidelines for Residual Solvents, Q3C(R3), (November 2005). Hydrates are formed when the solvent is water, or alcoholates are formed when the solvent is alcohol.

The terms “effective amount” or “therapeutically effective amount” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in a disease. An appropriate “effective” amount in any individual case may be determined using techniques, such as a dose escalation study.

The terms “subject,” “individual,” and “patient” are used interchangeably herein to refer to a mammal. Mammals include, but are not limited to, murines, simians, humans, farm animals, sport animals, and pets.

The term “peripherally restricted FAAH inhibitor” as used herein, refers to a fatty acid amide hydrolase (FAAH) inhibitor that inhibits FAAH to a greater extent in the periphery than in the central nervous system from a systemic dose. In some embodiments, the peripherally restricted FAAH inhibitor is 60% peripherally restricted. In some embodiments, the peripherally restricted FAAH inhibitor is 70% peripherally restricted. In some embodiments, the peripherally restricted FAAH inhibitor is 80% peripherally restricted. In some embodiments, the peripherally restricted FAAH inhibitor is 90% peripherally restricted. In some embodiments, the peripherally restricted FAAH inhibitor is 95% peripherally restricted.

The term “target in the brain” as used herein, refers to a biological target wherein the biological target is activated in the brain, but the target itself is present in the CNS or both the CNS and periphery. In some embodiments, the target is in the CNS. In some embodiments, the target is in the CNS and periphery. As a result of target activation in the brain, the activated target may elicit a biological effect in the CNS, periphery, or both the CNS and periphery. In some embodiments, the activated target elicits a biological effect in the CNS. In some embodiments, the activated target elicits a biological effect predominantly in the CNS. In some embodiments, the activated target elicits a biological effect in the periphery. In some embodiments, the activated target elicits a biological effect in both the CNS and periphery. In some embodiments, the term “target in the brain” refers to a target in a mammal brain. In some embodiments, the term “target in the brain” refers to a target in a mammal brain, wherein the mammal is a human.

### Targets

### S1P1

Sphingosine-1-phosphate receptor 1 (S1P receptor 1 or S1P1), also known as endothelial differentiation gene 1 (EDG1) is a Class A G-protein coupled receptor expressed on lymphocytes, neural cells, and the endothelium. There are five GPCRs (S1P1-5) in the family which recognize sphingolipid shinosine-1phosphate (S1P) and perform a variety of functions. S1P1 regulates vascular development and lymphocyte trafficking and agonists thereof have been approved for the treatment of relapsing forms of multiple sclerosis. Sustained activation (agonism) of S1P1 expressed on lymphocytes results in receptor internalization and proteasomal degradation, resulting in “functional antagonism” of S1P1. It has been reported that S1P1 agonists that can efficiently penetrate the CNS can induce receptor signaling and degradation of S1P1 expressed on neurons and astrocytes, resulting in reduced disease severity in experimental autoimmune encephalomyelitis (EAE) in mice.

### LPA1

Lysophosphatidic acid receptor 1 (LPA1) is a G protein-coupled receptor that binds extracellular lysophosphatidic acid (LPA) activating second messenger pathways and eliciting a number of cellular responses that regulate cellular activity, cell motility, cytoskeletal rearrangement and cell growth. LPA1 activation induces microglial activation in the CNS and the receptor is a key regulator of neuroinflammation. LPA1 activation also plays a key role in the induction of demyelination. Inhibition of LPA1 activity in the CNS may have beneficial effects in multiple CNS diseases which involve neuroinflammation and demyelination.

### GPR120

GPR120/FFAR4 is a receptor of unsaturated long-chain fatty acids expressed in macrophages, eosinophils, and adipose tissue reported to mediate anti-inflammatory mechanisms. In C₅₇BL/6 models of middle cerebral artery occlusion (MCAO) and an in vitro model of oxygen-glucose deprivation (OGD), increased GPR120 expression was observed in microglia and neurons following MCAO-induced ischemia. Treatment with RAR agonists inhibited OGD-induced inflammatory response in primary microglia and murine microglial BV2 cells, whereas silencing of GPR120 strongly exacerbated the inflammation induced by OGD and abolished the anti-inflammatory effects. Additionally, knockdown of GPR120 impaired the antiapoptotic effect of RAR agonists in OGD-induced rat pheochromocytoma (PC12) cells. GPR120 activation has also been reported to protect against focal cerebral ischemic injury by preventing inflammation and apoptosis.

### PGI2 Analogues or Prostaglandin I2 (IP) Receptor Agonists

Prostacyclin (prostaglandin I₂) is a metabolite of arachidonic acid or prostaglandin endoperoxides and is released from vascular endothelial cells. The I-type prostaglandin receptor (IP3 receptor) is a G protein-coupled receptor that is coupled to the activation of adenylate cyclase, which catalyzes the formation of 3′,5′ cyclic adenosine monophosphate (cAMP), a second messenger involved in vascular tone. IP receptors are expressed on vascular smooth muscle and platelets and serve as a vasorelaxant in smooth muscle and an inhibitor of platelet aggregation. Prostacyclin has also been reported to promote axonal remodeling of injured neuronal networks after CNS inflammation. Additionally, studies under pathological conditions revealed that after occlusion of the middle cerebral artery, a stable analog of prostacyclin reduced brain edema. It is thought that prostacyclin signaling directly acts on endothelial cells and enhances endothelial barrier function, reducing edema formation. Signaling in perivascular cells, such as pericytes, contributes to reducing capillary hydraulic permeability under pathological conditions in the adult CNS. Prostacyclin receptor signaling inhibitors have been reported to impair motor recovery, IP receptor agonists promote axonal remodeling and motor recovery after the induction of EAE. These findings revealed that angiogenesis plays an important role in neuronal rewiring and suggest that prostacyclin is a promising molecule for enhancing functional recovery in CNS diseases.

### TTR Stabilizer Analogues

Transthyretin (TTR) is a homo-tetramer composed of 127 amino acid subunits that carries thyroxine and holo-retinol binding protein (holo-RBP) in the blood. It is secreted by liver into the blood at a steady state concentration of about 3-6 μM and by the choroid plexus (CP) into the cerebrospinal fluid (CSF) at a steady state concentration of approx. 300 nM. Misfolding, aggregation, and deposition (amyloidogenesis) of TTR is linked to amyloid diseases, including senile systemic amyloidosis, familial amyloid polyneuropathy or cardiomyopathy. The TTR tetramer is non-amyloidogenic, but undergoes dissociation, monomer misfolding, and misassembly into numerous aggregated structures including amyloid under partially denaturing conditions. TTR has also been reported to counteract the neurotoxic effects of A3 peptides by reducing their aggregation and enhancing clearance of the oligomers and plaques in the brain.

### Pharmaceutical Compositions and Compounds

In some embodiments described herein is a pharmaceutical composition comprising a fatty acid amide hydrolase (FAAH) cleavable prodrug of Formula (I), or a pharmaceutically acceptable salt or solvate thereof:

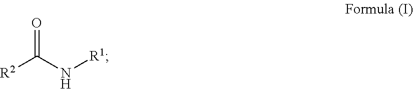

wherein:
R¹ is an amide prodrug moiety, wherein the prodrug of Formula (I) is enzymatically cleaved by fatty acid amide hydrolase (FAAH) in the brain to release

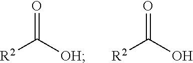

is a moiety that modulates a target in the brain; and a pharmaceutically acceptable excipient; further comprising a peripherally restricted FAAH inhibitor.

In some embodiments, the target is selected from sphingosine-1-phosphate receptor 1 (S1P1), lysophosphatidic acid receptor 1 (LPA1), G-protein coupled receptor 120 (GPR120), prostacyclin (PGI2), and transthyretin (TTR).

In some embodiments, the target is S1P1. In some embodiments, the target is S1P1, wherein R² is selected from

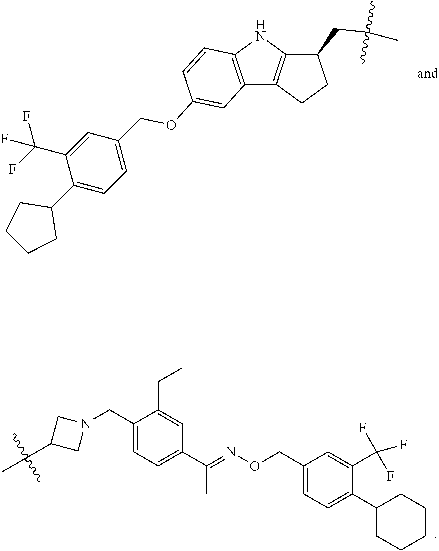 [SMILES: *C[C@H]1CCc2c1[nH]c1ccc(OCc3ccc(C4CCCC4)c(C(F)(F)F)c3)cc21 ; *C1CN(Cc2ccc(/C(C)=N/OCc3ccc(C4CCCCC4)c(C(F)(F)F)c3)cc2CC)C1]

In some embodiments, the target is LPA1. In some embodiments, the target is LPA1, wherein R² is selected from

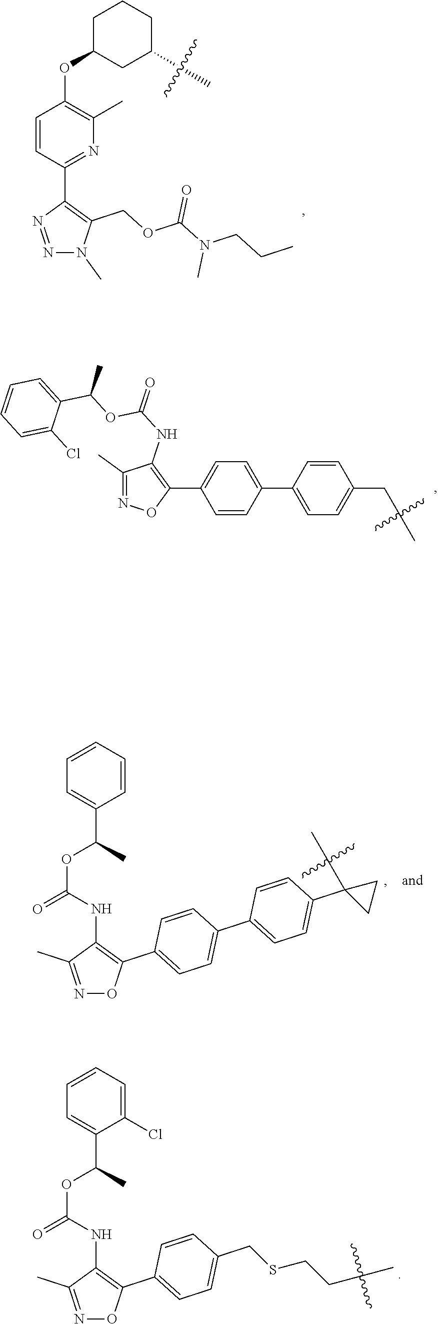

In some embodiments, the target is GPR120. In some embodiments, the target is GPR120, wherein R² is

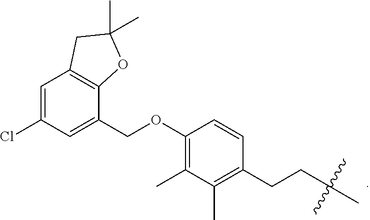 [SMILES: *CCc1ccc(OCc2cc(Cl)cc3c2OC(C)(C)C3)c(C)c1C]

In some embodiments, the target is TTR. In some embodiments, the target is TTR, wherein R² is selected from

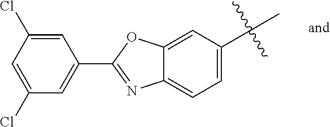 [SMILES: *c1ccc2nc(-c3cc(Cl)cc(Cl)c3)oc2c1]

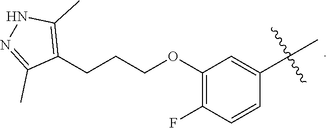 [SMILES: *c1ccc(F)c(OCCCc2c(C)n[nH]c2C)c1]

In some embodiments, the target is PGI2. In some embodiments, the target is PGI2, wherein R² is

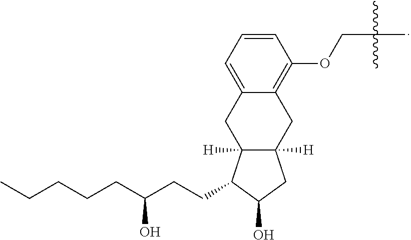

In some embodiments, R¹ is optionally substituted C₁₋₆alkyl. In some embodiments, R¹ is C₁₋₆alkyl optionally substituted with 1, 2, 3, 4, or 5 groups independently selected from halogen, —CN, —OH, —NH₂, —N(H)(C₁₋₆alkyl), N(C₁₋₆alkyl)₂, —C(O)OH, —C(O)O—C₁₋₆alkyl, —C(O)NH₂, —C(O)N(H)(C₁₋₆alkyl), —C(O)N(C₁₋₆alkyl)₂, —C(O)C₁₋₆alkyl, —S(O)₂C₁₋₆alkyl, —S(O)₂NH₂, —S(O)₂N(H)(C₁₋₆alkyl), —S(O)₂N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments, R¹ is C₁₋₆ alkyl optionally substituted with 1, 2, 3, 4, or 5 groups independently selected from halogen, —OH, —NH₂, —N(H)(C₁₋₆alkyl), N(C₁₋₆alkyl)₂, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments, R¹ is C₁₋₆alkyl optionally substituted with 1, 2, 3, 4, or 5 groups independently selected from halogen, —OH, —NH₂, —N(H)(C₁₋₆alkyl), N(C₁₋₆alkyl)₂, C₁₋₆haloalkyl, and C₁₋₆alkoxy. In some embodiments, R¹ is unsubstituted C₁₋₆alkyl.

In some embodiments, R¹ is optionally substituted C₃₋₆cycloalkyl. In some embodiments, R² is C₃₋₆cycloalkyl optionally substituted with 1, 2, 3, 4, or 5 groups independently selected from halogen, —CN, —OH, —NH₂, —N(H)(C₁₋₆alkyl), N(C₁₋₆alkyl)₂, —C(O)OH, —C(O)O—C₁₋₆alkyl, —C(O)NH₂, —C(O)N(H)(C₁₋₆alkyl), —C(O)N(C₁₋₆alkyl)₂, —C(O)C₁₋₆alkyl, —S(O)₂C₁₋₆alkyl, —S(O)₂NH₂, —S(O)₂N(H)(C₁₋₆alkyl), —S(O)₂N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments, R¹ is C₃₋₆cycloalkyl optionally substituted with 1, 2, 3, 4, or 5 groups independently selected from halogen, —OH, —NH₂, —N(H)(C₁₋₆alkyl), N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments, R¹ is C₃₋₆cycloalkyl optionally substituted with 1, 2, 3, 4, or 5 groups independently selected from halogen, —OH, —NH₂, —N(H)(C₁₋₆alkyl), N(C₁₋₆alkyl)₂, C₁₋₆alkyl, C₁₋₆ haloalkyl, and C₁₋₆alkoxy. In some embodiments, R¹ is unsubstituted C₃₋₆cycloalkyl.

In some embodiments, R¹ is optionally substituted C₂₋₉heterocycloalkyl. In some embodiments, R¹ is C₂₋₉heterocycloalkyl optionally substituted with 1, 2, 3, 4, or 5 groups independently selected from halogen, —CN, —OH, —NH₂, —N(H)(C₁₋₆alkyl), N(C₁₋₆alkyl)₂, —C(O)OH, —C(O)O—C₁₋₆alkyl, —C(O)NH₂, —C(O)N(H)(C₁₋₆alkyl), —C(O)N(C₁₋₆alkyl)₂, —C(O)C₁₋₆alkyl, —S(O)₂C₁₋₆alkyl, —S(O)₂NH₂, —S(O)₂N(H)(C₁₋₆alkyl), —S(O)₂N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments, R¹ is C₂₋₉heterocycloalkyl optionally substituted with 1, 2, 3, 4, or 5 groups independently selected from halogen, —OH, —NH₂, —N(H)(C₁₋₆alkyl), N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments, R¹ is C₂₋₉heterocycloalkyl optionally substituted with 1, 2, 3, 4, or 5 groups independently selected from halogen, —OH, —NH₂, —N(H)(C₁₋₆alkyl), N(C₁₋₆alkyl)₂, C₁₋₆alkyl, C₁₋₆ haloalkyl, and C₁₋₆alkoxy. In some embodiments, R¹ is unsubstituted C₂₋₉heterocycloalkyl.

In some embodiments, R¹ is optionally substituted C₆₋₁₀aryl. In some embodiments, R¹ is C₆₋₁₀aryl optionally substituted with 1, 2, 3, 4, or 5 groups independently selected from halogen, —CN, —OH, —NH₂, —N(H)(C₁₋₆alkyl), N(C₁₋₆alkyl)₂, —C(O)OH, —C(O)O—C₁₋₆alkyl, —C(O)NH₂, —C(O)N(H)(C₁₋₆alkyl), —C(O)N(C₁₋₆alkyl)₂, —C(O)C₁₋₆alkyl, —S(O)₂C₁₋₆alkyl, —S(O)₂NH₂, —S(O)₂N(H)(C₁₋₆alkyl), —S(O)₂N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments, R¹ is C₆₋₁₀aryl optionally substituted with 1, 2, 3, 4, or 5 groups independently selected from halogen, —OH, —NH₂, —N(H)(C₁₋₆alkyl), N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments, R¹ is C₆₋₁₀aryl optionally substituted with 1, 2, 3, 4, or 5 groups independently selected from halogen, —OH, —NH₂, —N(H)(C₁₋₆alkyl), N(C₁₋₆alkyl)₂, C₁₋₆alkyl, C₁₋₆haloalkyl, and C₁₋₆alkoxy. In some embodiments, R¹ is unsubstituted C₆₋₁₀aryl.

In some embodiments, R¹ is optionally substituted C₁₋₉heteroaryl. In some embodiments, R¹ is C₁₋₉heteroaryl optionally substituted with 1, 2, 3, 4, or 5 groups independently selected from halogen, —CN, —OH, —NH₂, —N(H)(C₁₋₆alkyl), N(C₁₋₆alkyl)₂, —C(O)OH, —C(O)O—C₁₋₆alkyl, —C(O)NH₂, —C(O)N(H)(C₁₋₆alkyl), —C(O)N(C₁₋₆alkyl)₂, —C(O)C₁₋₆alkyl, —S(O)₂C₁₋₆alkyl, —S(O)₂NH₂, —S(O)₂N(H)(C₁₋₆alkyl), —S(O)₂N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments, R¹ is C₁₋₉heteroaryl optionally substituted with 1, 2, 3, 4, or 5 groups independently selected from halogen, —OH, —NH₂, —N(H)(C₁₋₆alkyl), N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments, R¹ is C₁₋₉heteroaryl optionally substituted with 1, 2, 3, 4, or 5 groups independently selected from halogen, —OH, —NH₂, —N(H)(C₁₋₆alkyl), N(C₁₋₆alkyl)₂, C₁₋₆alkyl, C₁₋₆ haloalkyl, and C₁₋₆alkoxy. In some embodiments, R¹ is unsubstituted C₁₋₉heteroaryl.

In some embodiments, R¹ is —CH₃. In some embodiments, R¹ is —CH₂CH₃. In some embodiments, R¹ is —CH₂CH₂CH₃. In some embodiments, R¹ is —CHCH₃)₂. In some embodiments, R¹ is

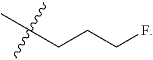 [SMILES: *CCCF]

In some embodiments, R¹ is

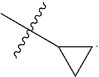 [SMILES: *C1CC1]

In some embodiments, R¹ is

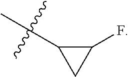 [SMILES: *C1CC1F]

In some embodiments, R¹ is

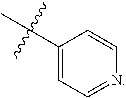 [SMILES: *c1ccncc1]

In some embodiments, R¹ is

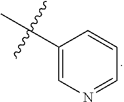 [SMILES: *c1cccnc1]

In some embodiments, R¹ is

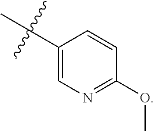 [SMILES: *c1ccc(OC)nc1]

In some embodiments, R¹ is

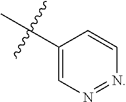 [SMILES: *c1ccnnc1]

In some embodiments, R¹ is

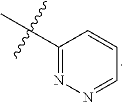 [SMILES: *c1cccnn1]

In some embodiments, R¹ is

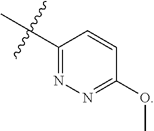 [SMILES: *c1ccc(OC)nn1]

In some embodiments, R¹ is

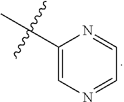 [SMILES: *c1cnccn1]

In some embodiments, R¹ is

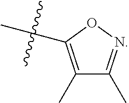 [SMILES: *c1onc(C)c1C]

In some embodiments, the fatty acid amide hydrolase (FAAH) cleavable prodrug of Formula (I) is selected from:

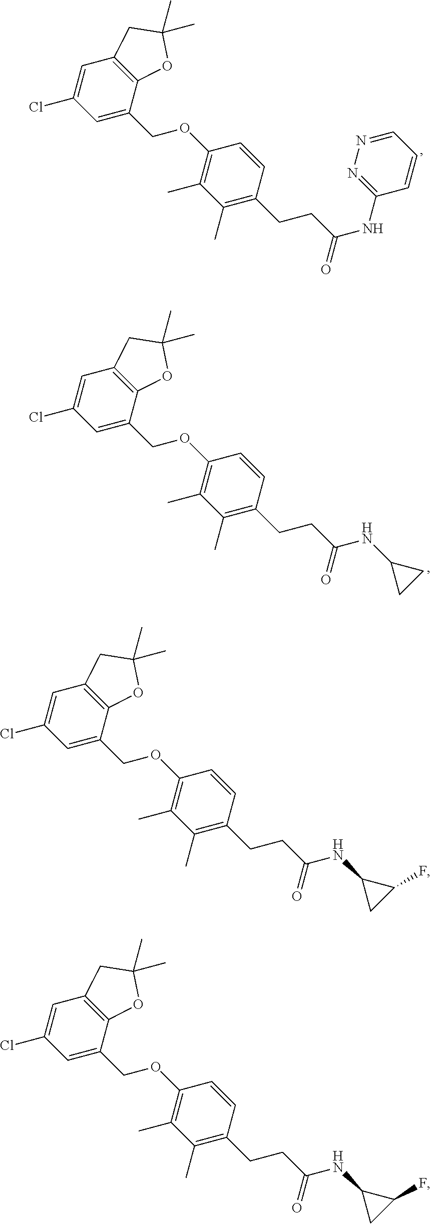 [SMILES: Cc1c(CCC(=O)Nc2cccnn2)ccc(OCc2cc(Cl)cc3c2OC(C)(C)C3)c1C ; Cc1c(CCC(=O)NC2CC2)ccc(OCc2cc(Cl)cc3c2OC(C)(C)C3)c1C ; Cc1c(CCC(=O)N[C@@H]2C[C@H]2F)ccc(OCc2cc(Cl)cc3c2OC(C)(C)C3)c1C ; Cc1c(CCC(=O)N[C@@H]2C[C@@H]2F)ccc(OCc2cc(Cl)cc3c2OC(C)(C)C3)c1C]

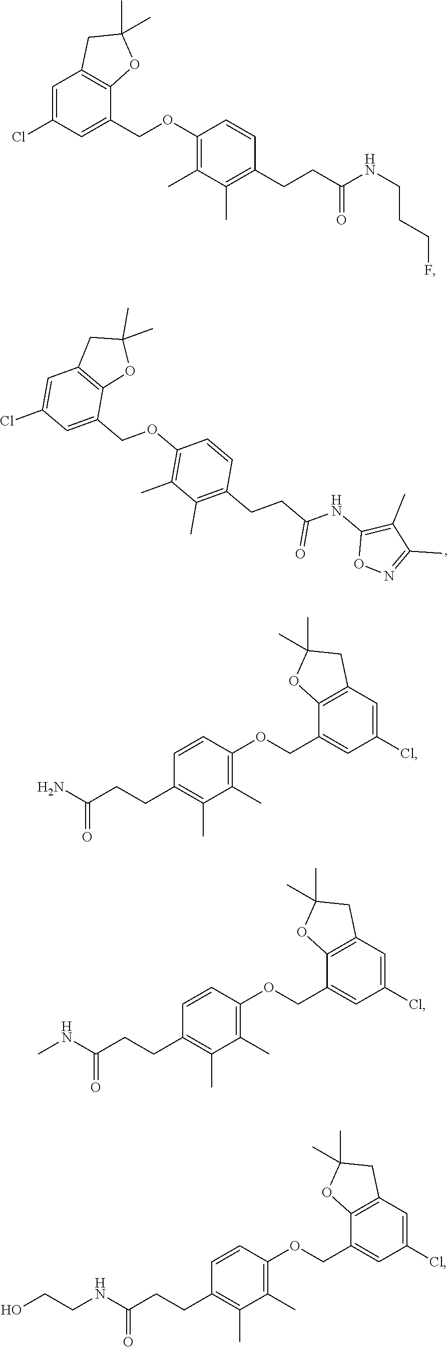

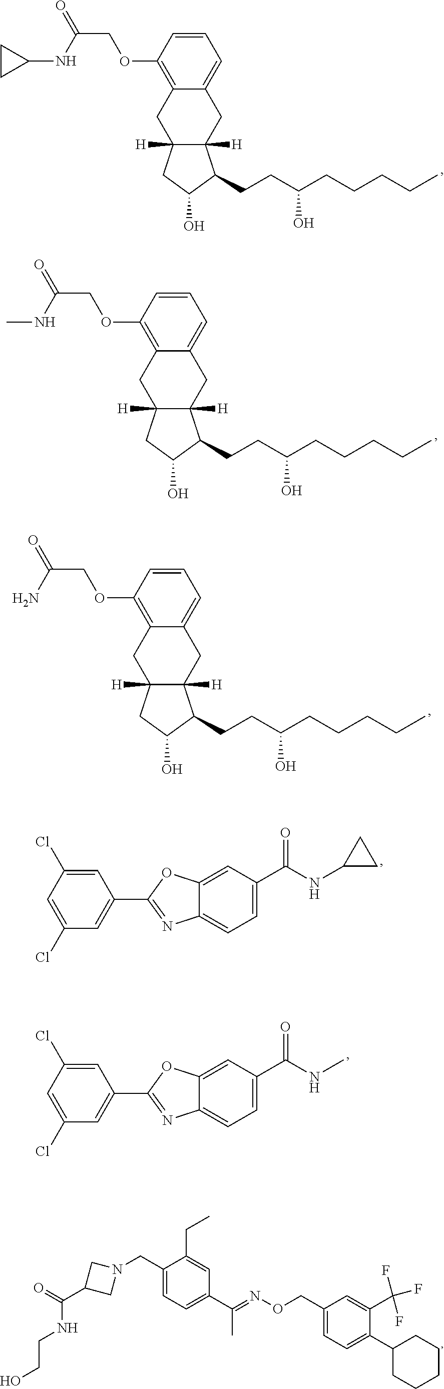

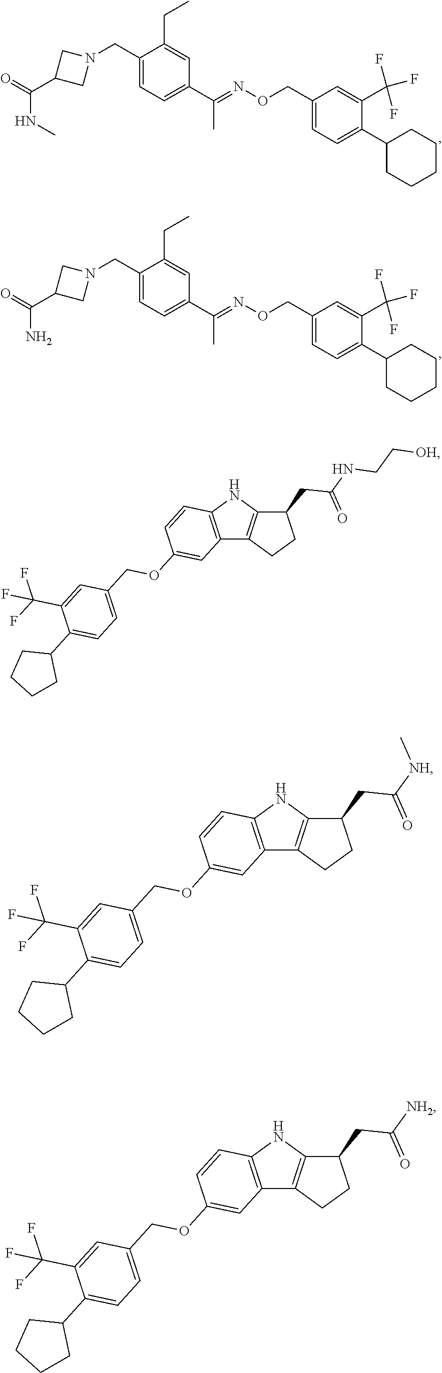 [SMILES: CCc1cc(/C(C)=N/OCc2ccc(C3CCCCC3)c(C(F)(F)F)c2)ccc1CN1CC(C(=O)NC)C1 ; CCc1cc(/C(C)=N/OCc2ccc(C3CCCCC3)c(C(F)(F)F)c2)ccc1CN1CC(C(N)=O)C1 ; O=C(C[C@H]1CCc2c1[nH]c1ccc(OCc3ccc(C4CCCC4)c(C(F)(F)F)c3)cc21)NCCO ; CNC(=O)C[C@H]1CCc2c1[nH]c1ccc(OCc3ccc(C4CCCC4)c(C(F)(F)F)c3)cc21 ; NC(=O)C[C@H]1CCc2c1[nH]c1ccc(OCc3ccc(C4CCCC4)c(C(F)(F)F)c3)cc21]

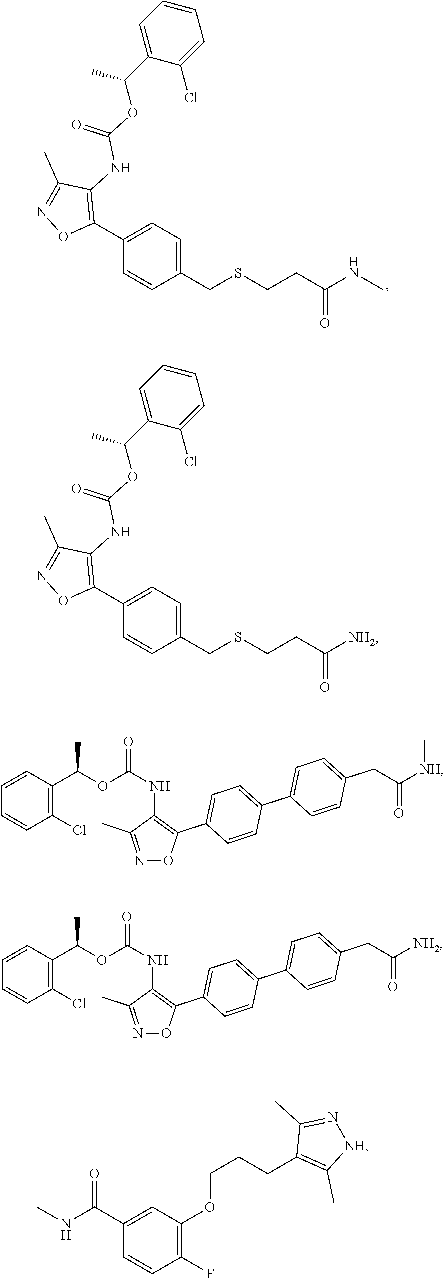

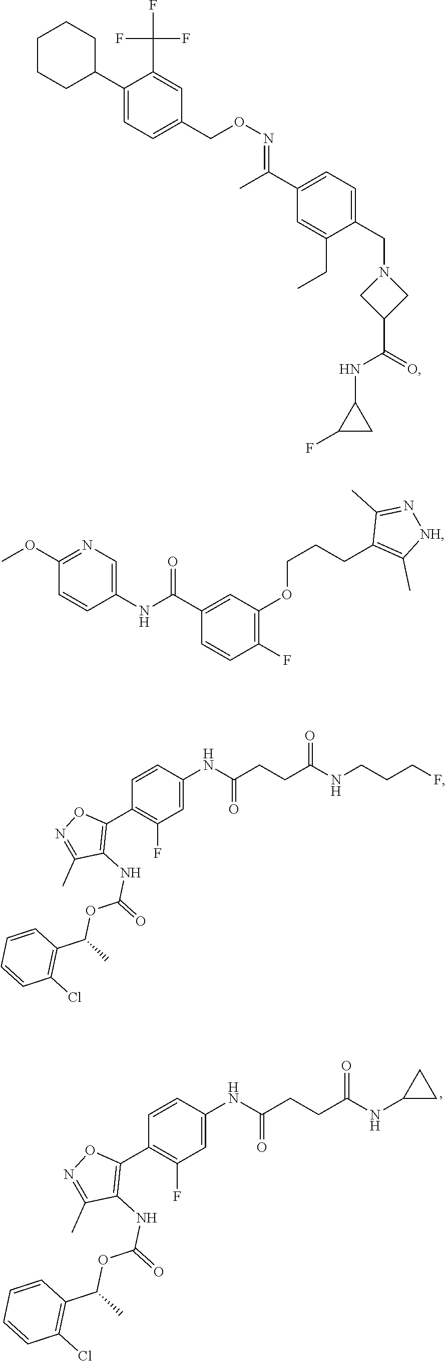 [SMILES: CCc1cc(/C(C)=N/OCc2ccc(C3CCCCC3)c(C(F)(F)F)c2)ccc1CN1CC(C(=O)NC2CC2F)C1 ; COc1ccc(NC(=O)c2ccc(F)c(OCCCc3c(C)n[nH]c3C)c2)cn1 ; Cc1noc(-c2ccc(NC(=O)CCC(=O)NCCCF)cc2F)c1NC(=O)O[C@H](C)c1ccccc1Cl ; Cc1noc(-c2ccc(NC(=O)CCC(=O)NC3CC3)cc2F)c1NC(=O)O[C@H](C)c1ccccc1Cl]

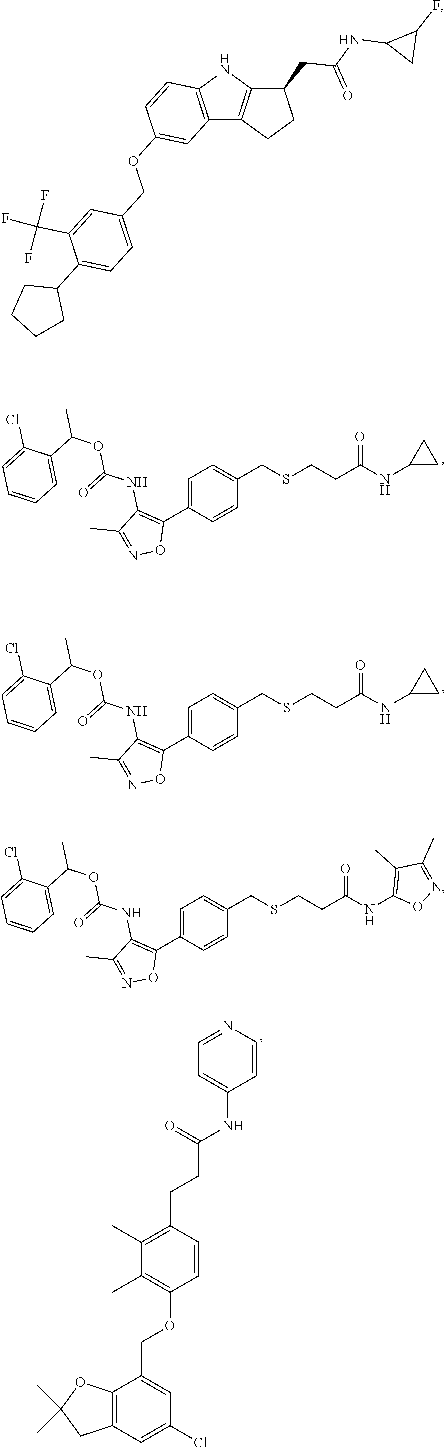 [SMILES: O=C(C[C@H]1CCc2c1[nH]c1ccc(OCc3ccc(C4CCCC4)c(C(F)(F)F)c3)cc21)NC1CC1F ; Cc1noc(-c2ccc(CSCCC(=O)NC3CC3)cc2)c1NC(=O)OC(C)c1ccccc1Cl ; Cc1noc(-c2ccc(CSCCC(=O)NC3CC3)cc2)c1NC(=O)OC(C)c1ccccc1Cl ; Cc1noc(NC(=O)CCSCc2ccc(-c3onc(C)c3NC(=O)OC(C)c3ccccc3Cl)cc2)c1C ; Cc1c(CCC(=O)Nc2ccncc2)ccc(OCc2cc(Cl)cc3c2OC(C)(C)C3)c1C]

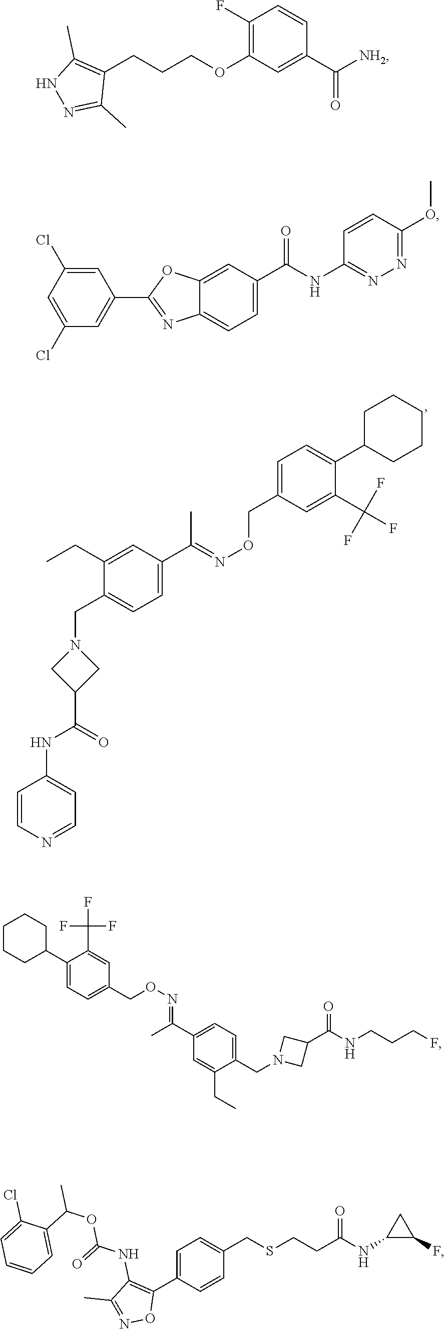 [SMILES: Cc1n[nH]c(C)c1CCCOc1cc(C(N)=O)ccc1F ; COc1ccc(NC(=O)c2ccc3nc(-c4cc(Cl)cc(Cl)c4)oc3c2)nn1 ; CCc1cc(/C(C)=N/OCc2ccc(C3CCCCC3)c(C(F)(F)F)c2)ccc1CN1CC(C(=O)Nc2ccncc2)C1 ; CCc1cc(/C(C)=N/OCc2ccc(C3CCCCC3)c(C(F)(F)F)c2)ccc1CN1CC(C(=O)NCCCF)C1 ; Cc1noc(-c2ccc(CSCCC(=O)N[C@@H]3C[C@H]3F)cc2)c1NC(=O)OC(C)c1ccccc1Cl]

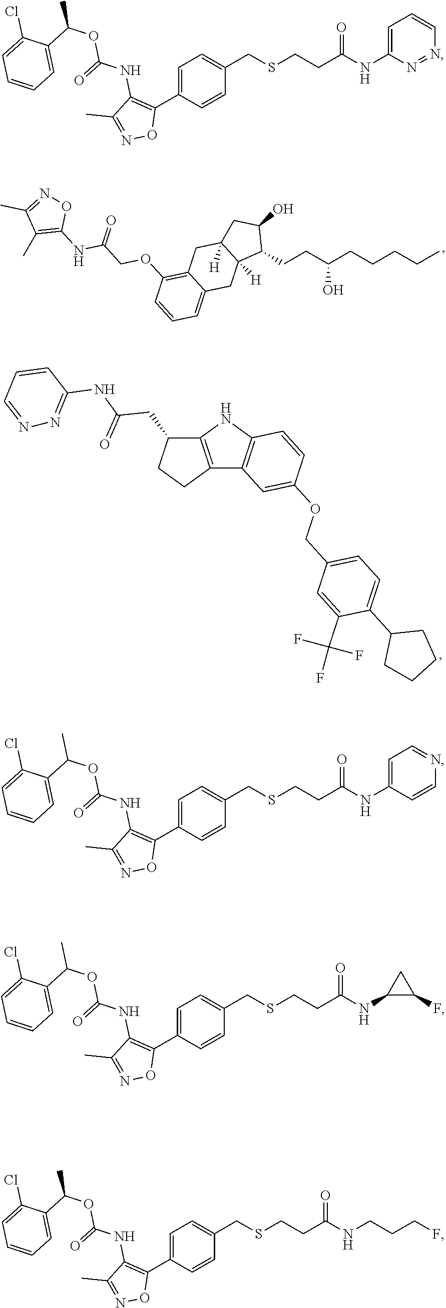

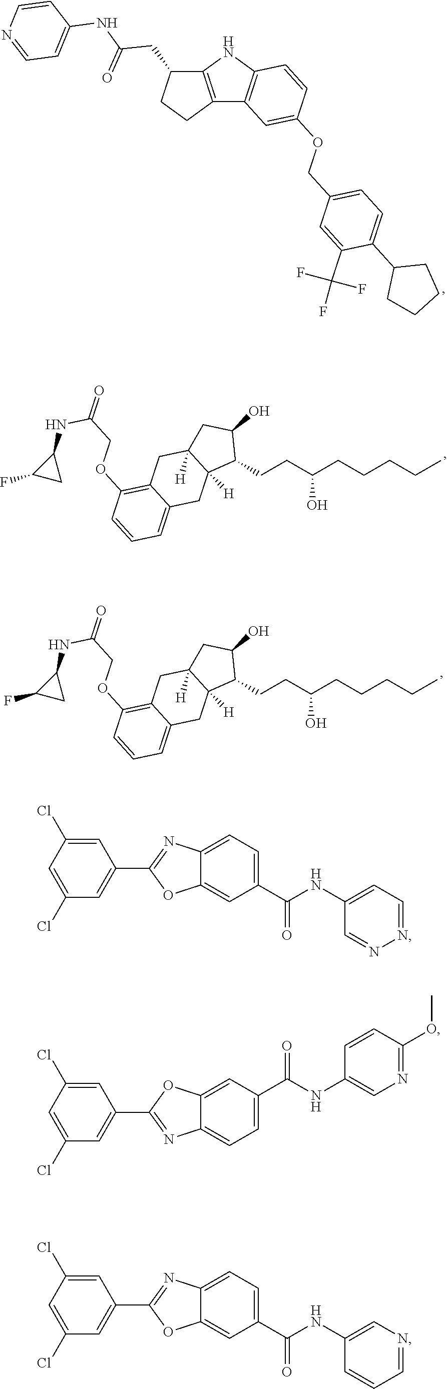

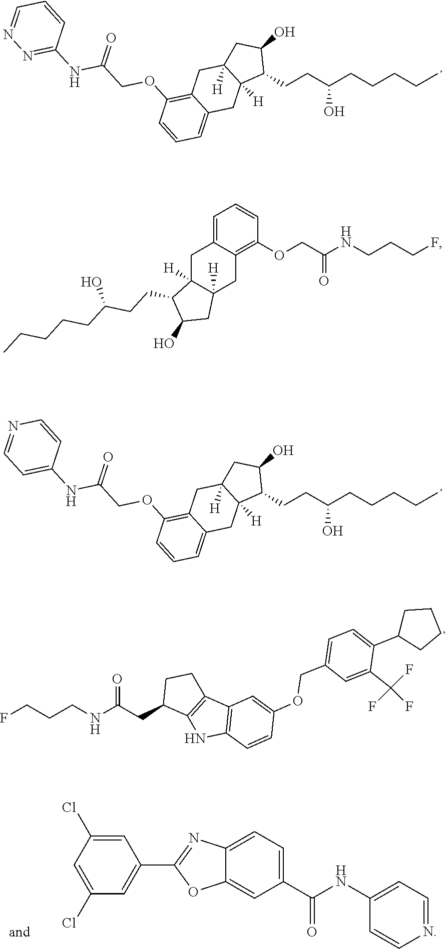

In some embodiments of the pharmaceutical compositions described herein, the peripherally restricted FAAH inhibitor is ASP-3652.

In some embodiments described herein is a compound, or a pharmaceutically acceptable salt or solvate thereof, of Formula (II):

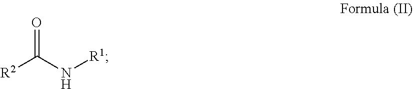

wherein:
R¹ is an amide prodrug moiety, wherein the amide prodrug moiety is enzymatically cleaved by fatty acid amide hydrolase (FAAH) in the brain to release

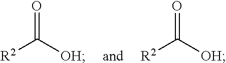

is a moiety that modulates S1P1 in the brain.

In some embodiments is a compound of Formula (II), wherein R² is selected from

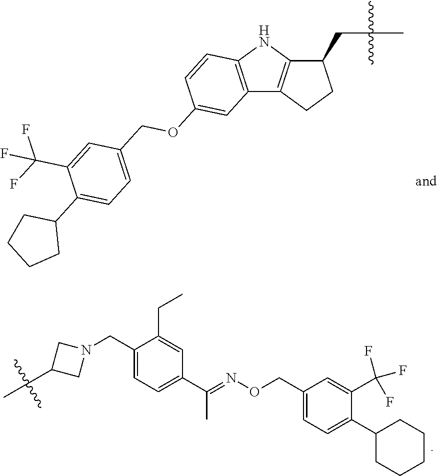 [SMILES: *C[C@H]1CCc2c1[nH]c1ccc(OCc3ccc(C4CCCC4)c(C(F)(F)F)c3)cc21 ; *C1CN(Cc2ccc(/C(C)=N/OCc3ccc(C4CCCCC4)c(C(F)(F)F)c3)cc2CC)C1]

In some embodiments is a compound of Formula (II), wherein R² is

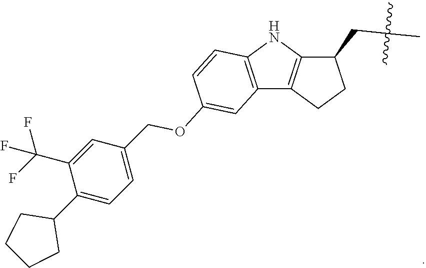 [SMILES: *C[C@H]1CCc2c1[nH]c1ccc(OCc3ccc(C4CCCC4)c(C(F)(F)F)c3)cc21]

In some embodiments is a compound of Formula (II), wherein R² is

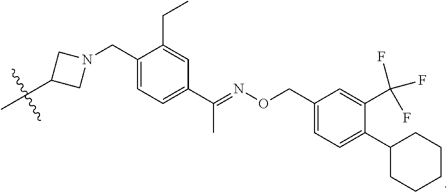 [SMILES: *C1CN(Cc2ccc(/C(C)=N/OCc3ccc(C4CCCCC4)c(C(F)(F)F)c3)cc2CC)C1]

In some embodiments is a compound of Formula (II), wherein R¹ is optionally substituted C₁₋₆alkyl. In some embodiments is a compound of Formula (II), wherein R¹ is C₁₋₆ alkyl optionally substituted with 1, 2, 3, 4, or 5 groups independently selected from halogen, —CN, —OH, —NH₂, —N(H)(C₁₋₆alkyl), N(C₁₋₆alkyl)₂, —C(O)OH, —C(O)O—C₁₋₆alkyl, —C(O)NH₂, —C(O)N(H)(C₁₋₆alkyl), —C(O)N(C₁₋₆alkyl)₂, —C(O)C₁₋₆alkyl, —S(O)₂C₁₋₆alkyl, —S(O)₂NH₂, —S(O)₂N(H)(C₁₋₆alkyl), —S(O)₂N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments is a compound of Formula (II), wherein R¹ is C₁₋₆alkyl optionally substituted with 1, 2, 3, 4, or 5 groups independently selected from halogen, —OH, —NH₂, —N(H)(C₁₋₆alkyl), N(C₁₋₆alkyl)₂, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments is a compound of Formula (II), wherein R¹ is C₁₋₆alkyl optionally substituted with 1, 2, 3, 4, or 5 groups independently selected from halogen, —OH, —NH₂, —N(H)(C₁₋₆alkyl), N(C₁₋₆alkyl)₂, C₁₋₆haloalkyl, and C₁₋₆alkoxy. In some embodiments is a compound of Formula (II), wherein R¹ is unsubstituted C₁₋₆alkyl.

In some embodiments is a compound of Formula (II), wherein R¹ is optionally substituted C₃₋₆cycloalkyl. In some embodiments is a compound of Formula (II), wherein R¹ is C₃₋₆cycloalkyl optionally substituted with 1, 2, 3, 4, or 5 groups independently selected from halogen, —CN, —OH, —NH₂, —N(H)(C₁₋₆alkyl), N(C₁₋₆alkyl)₂, —C(O)OH, —C(O)O—C₁₋₆alkyl, —C(O)NH₂, —C(O)N(H)(C₁₋₆alkyl), —C(O)N(C₁₋₆alkyl)₂, —C(O)C₁₋₆alkyl, —S(O)₂C₁₋₆alkyl, —S(O)₂NH₂, —S(O)₂N(H)(C₁₋₆alkyl), —S(O)₂N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments is a compound of Formula (II), wherein R¹ is C₃₋₆cycloalkyl optionally substituted with 1, 2, 3, 4, or 5 groups independently selected from halogen, —OH, —NH₂, —N(H)(C₁₋₆alkyl), N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉ heteroaryl. In some embodiments is a compound of Formula (II), wherein R¹ is C₃₋₆cycloalkyl optionally substituted with 1, 2, 3, 4, or 5 groups independently selected from halogen, —OH, —NH₂, —N(H)(C₁₋₆alkyl), N(C₁₋₆alkyl)₂, C₁₋₆alkyl, C₁₋₆haloalkyl, and C₁₋₆alkoxy. In some embodiments is a compound of Formula (II), wherein R¹ is unsubstituted C₃₋₆cycloalkyl.

In some embodiments is a compound of Formula (II), wherein R¹ is optionally substituted C₂₋₉heterocycloalkyl. In some embodiments is a compound of Formula (II), wherein R² is C₂₋₉heterocycloalkyl optionally substituted with 1, 2, 3, 4, or 5 groups independently selected from halogen, —CN, —OH, —NH₂, —N(H)(C₁₋₆alkyl), N(C₁₋₆alkyl)₂, —C(O)OH, —C(O)O—C₁₋₆alkyl, —C(O)NH₂, —C(O)N(H)(C₁₋₆alkyl), —C(O)N(C₁₋₆alkyl)₂, —C(O)C₁₋₆alkyl, —S(O)₂C₁₋₆alkyl, —S(O)₂NH₂, —S(O)₂N(H)(C₁₋₆alkyl), —S(O)₂N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments is a compound of Formula (II), wherein R¹ is C₂₋₉heterocycloalkyl optionally substituted with 1, 2, 3, 4, or 5 groups independently selected from halogen, —OH, —NH₂, —N(H)(C₁₋₆alkyl), N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉ heteroaryl. In some embodiments is a compound of Formula (II), wherein R¹ is C₂₋₉heterocycloalkyl optionally substituted with 1, 2, 3, 4, or 5 groups independently selected from halogen, —OH, —NH₂, —N(H)(C₁₋₆alkyl), N(C₁₋₆alkyl)₂, C₁₋₆alkyl, C₁₋₆haloalkyl, and C₁₋₆alkoxy.

In some embodiments is a compound of Formula (II), wherein R¹ is unsubstituted C₂₋₉heterocycloalkyl.

In some embodiments is a compound of Formula (II), wherein R¹ is optionally substituted C₆₋₁₀aryl. In some embodiments is a compound of Formula (II), wherein R¹ is C₆₋₁₀aryl optionally substituted with 1, 2, 3, 4, or 5 groups independently selected from halogen, —CN, —OH, —NH₂, —N(H)(C₁₋₆alkyl), N(C₁₋₆alkyl)₂, —C(O)OH, —C(O)O—C₁₋₆alkyl, —C(O)NH₂, —C(O)N(H)(C₁₋₆alkyl), —C(O)N(C₁₋₆alkyl)₂, —C(O)C₁₋₆alkyl, —S(O)₂C₁₋₆alkyl, —S(O)₂NH₂, —S(O)₂N(H)(C₁₋₆alkyl), —S(O)₂N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments is a compound of Formula (II), wherein R¹ is C₆₋₁₀aryl optionally substituted with 1, 2, 3, 4, or 5 groups independently selected from halogen, —OH, —NH₂, —N(H)(C₁₋₆alkyl), N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments is a compound of Formula (II), wherein R¹ is C₆₋₁₀aryl optionally substituted with 1, 2, 3, 4, or 5 groups independently selected from halogen, —OH, —NH₂, —N(H)(C₁₋₆alkyl), N(C₁₋₆alkyl)₂, C₁₋₆alkyl, C₁₋₆haloalkyl, and C₁₋₆alkoxy. In some embodiments is a compound of Formula (II), wherein R¹ is unsubstituted C₆₋₁₀aryl.

In some embodiments is a compound of Formula (II), wherein R¹ is optionally substituted C₁₋₉heteroaryl. In some embodiments is a compound of Formula (II), wherein R¹ is C₁₋₉heteroaryl optionally substituted with 1, 2, 3, 4, or 5 groups independently selected from halogen, —CN, —OH, —NH₂, —N(H)(C₁₋₆alkyl), N(C₁₋₆alkyl)₂, —C(O)OH, —C(O)O—C₁₋₆alkyl, —C(O)NH₂, —C(O)N(H)(C₁₋₆alkyl), —C(O)N(C₁₋₆alkyl)₂, —C(O)C₁₋₆alkyl, —S(O)₂C₁₋₆alkyl, —S(O)₂NH₂, —S(O)₂N(H)(C₁₋₆alkyl), —S(O)₂N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments is a compound of Formula (II), wherein R¹ is C₁₋₉heteroaryl optionally substituted with 1, 2, 3, 4, or 5 groups independently selected from halogen, —OH, —NH₂, —N(H)(C₁₋₆alkyl), N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉ heteroaryl. In some embodiments is a compound of Formula (II), wherein R¹ is C₁₋₉heteroaryl optionally substituted with 1, 2, 3, 4, or 5 groups independently selected from halogen, —OH, —NH₂, —N(H)(C₁₋₆alkyl), N(C₁₋₆alkyl)₂, C₁₋₆alkyl, C₁₋₆haloalkyl, and C₁₋₆alkoxy. In some embodiments is a compound of Formula (II), wherein R¹ is unsubstituted C₁₋₉heteroaryl.

In some embodiments is a compound of Formula (II), wherein R¹ is —CH₃. In some embodiments is a compound of Formula (II), wherein R¹ is —CH₂CH₃. In some embodiments is a compound of Formula (II), wherein R¹ is —CH₂CH₂CH₃. In some embodiments is a compound of Formula (II), wherein R² is —CHCH₃)₂. In some embodiments is a compound of Formula (II), wherein R¹ is

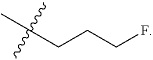 [SMILES: *CCCF]

In some embodiments is a compound of Formula (II), wherein R¹ is

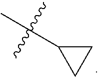 [SMILES: *C1CC1]

In some embodiments is a compound of Formula (II), wherein R¹ is

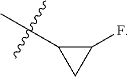 [SMILES: *C1CC1F]

In some embodiments is a compound of Formula (II), wherein R¹ is

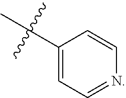 [SMILES: *c1ccncc1]

In some embodiments is a compound of Formula (II), wherein R¹ is

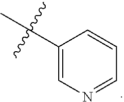 [SMILES: *c1cccnc1]

In some embodiments is a compound of Formula (II), wherein R¹ is

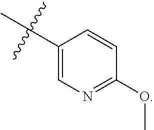 [SMILES: *c1ccc(OC)nc1]

In some embodiments is a compound of Formula (II), wherein R¹ is

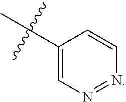 [SMILES: *c1ccnnc1]

In some embodiments is a compound of Formula (II), wherein R¹ is

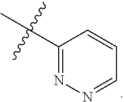 [SMILES: *c1cccnn1]

In some embodiments is a compound of Formula (II), wherein R¹ is

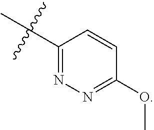 [SMILES: *c1ccc(OC)nn1]

In some embodiments is a compound of Formula (II), wherein R¹ is

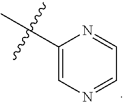 [SMILES: *c1cnccn1]

In some embodiments is a compound of Formula (II), wherein R¹ is

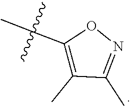 [SMILES: *c1onc(C)c1C]

In some embodiments the compound of Formula (II) is selected from:

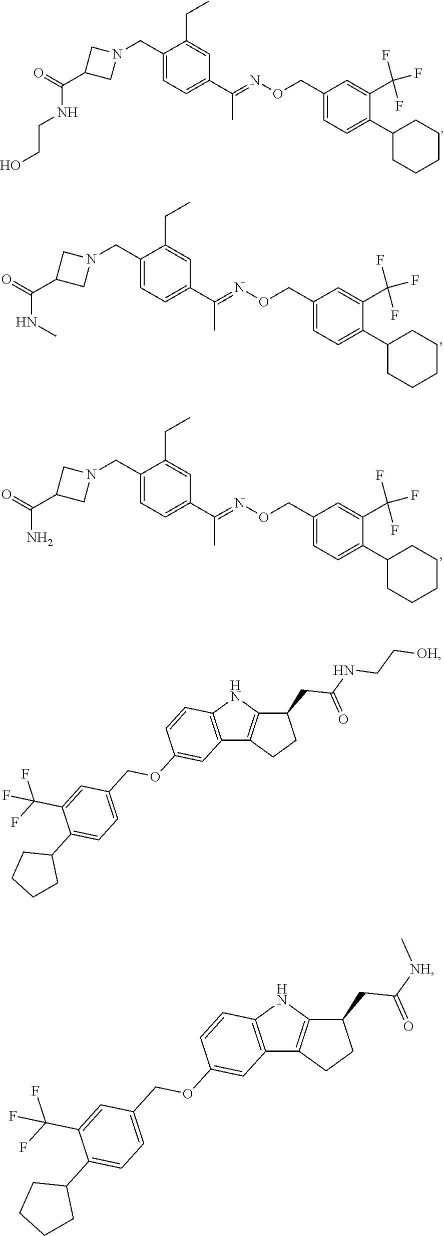 [SMILES: CCc1cc(/C(C)=N/OCc2ccc(C3CCCCC3)c(C(F)(F)F)c2)ccc1CN1CC(C(=O)NCCO)C1 ; CCc1cc(/C(C)=N/OCc2ccc(C3CCCCC3)c(C(F)(F)F)c2)ccc1CN1CC(C(=O)NC)C1 ; CCc1cc(/C(C)=N/OCc2ccc(C3CCCCC3)c(C(F)(F)F)c2)ccc1CN1CC(C(N)=O)C1 ; O=C(C[C@H]1CCc2c1[nH]c1ccc(OCc3ccc(C4CCCC4)c(C(F)(F)F)c3)cc21)NCCO ; CNC(=O)C[C@H]1CCc2c1[nH]c1ccc(OCc3ccc(C4CCCC4)c(C(F)(F)F)c3)cc21]

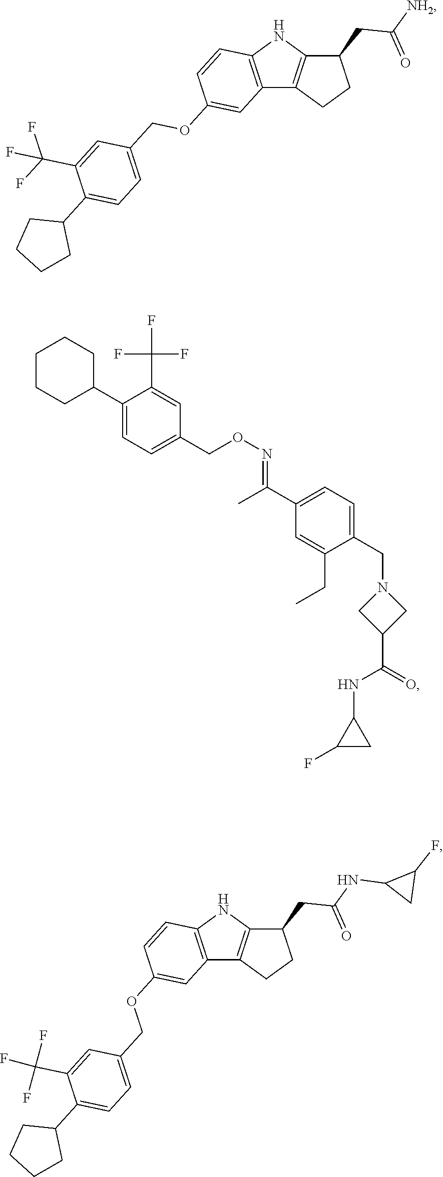 [SMILES: NC(=O)C[C@H]1CCc2c1[nH]c1ccc(OCc3ccc(C4CCCC4)c(C(F)(F)F)c3)cc21 ; CCc1cc(/C(C)=N/OCc2ccc(C3CCCCC3)c(C(F)(F)F)c2)ccc1CN1CC(C(=O)NC2CC2F)C1 ; O=C(C[C@H]1CCc2c1[nH]c1ccc(OCc3ccc(C4CCCC4)c(C(F)(F)F)c3)cc21)NC1CC1F]

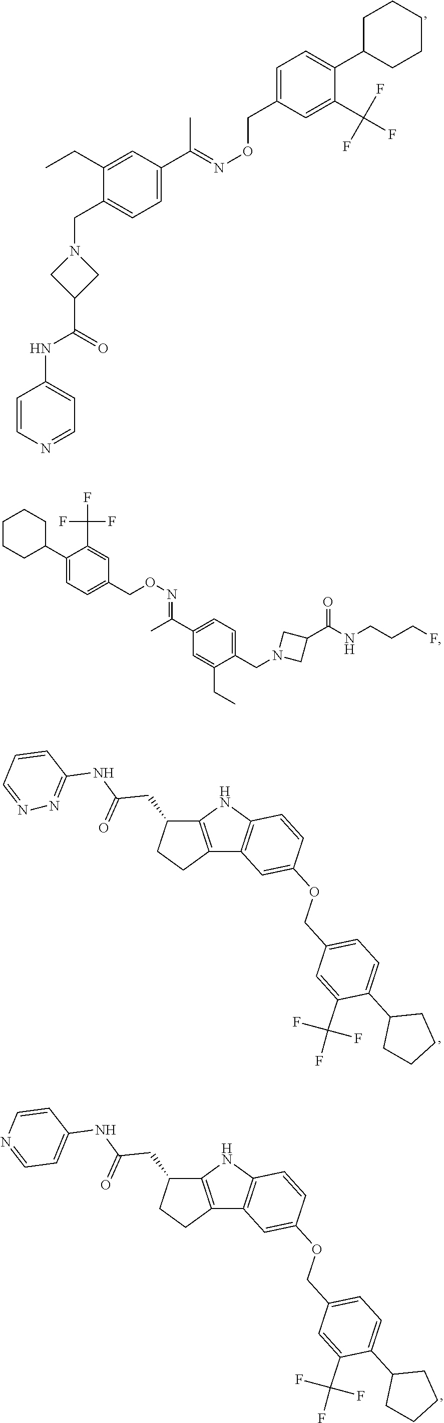 [SMILES: CCc1cc(/C(C)=N/OCc2ccc(C3CCCCC3)c(C(F)(F)F)c2)ccc1CN1CC(C(=O)Nc2ccncc2)C1 ; CCc1cc(/C(C)=N/OCc2ccc(C3CCCCC3)c(C(F)(F)F)c2)ccc1CN1CC(C(=O)NCCCF)C1 ; O=C(C[C@H]1CCc2c1[nH]c1ccc(OCc3ccc(C4CCCC4)c(C(F)(F)F)c3)cc21)Nc1cccnn1 ; O=C(C[C@H]1CCc2c1[nH]c1ccc(OCc3ccc(C4CCCC4)c(C(F)(F)F)c3)cc21)Nc1ccncc1]

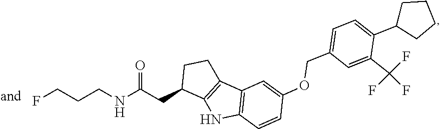 [SMILES: O=C(C[C@H]1CCc2c1[nH]c1ccc(OCc3ccc(C4CCCC4)c(C(F)(F)F)c3)cc21)NCCCF]

In some embodiments described herein is a compound, or a pharmaceutically acceptable salt or solvate thereof, of Formula (III):

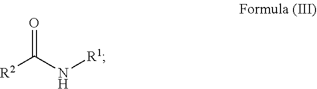

wherein:
R¹ is an amide prodrug moiety, wherein the amide prodrug moiety is enzymatically cleaved by fatty acid amide hydrolase (FAAH) in the brain to release

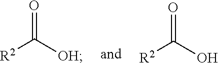

is a moiety that modulates LPA1 in the brain.

In some embodiments is a compound of Formula (III), wherein R² is selected from

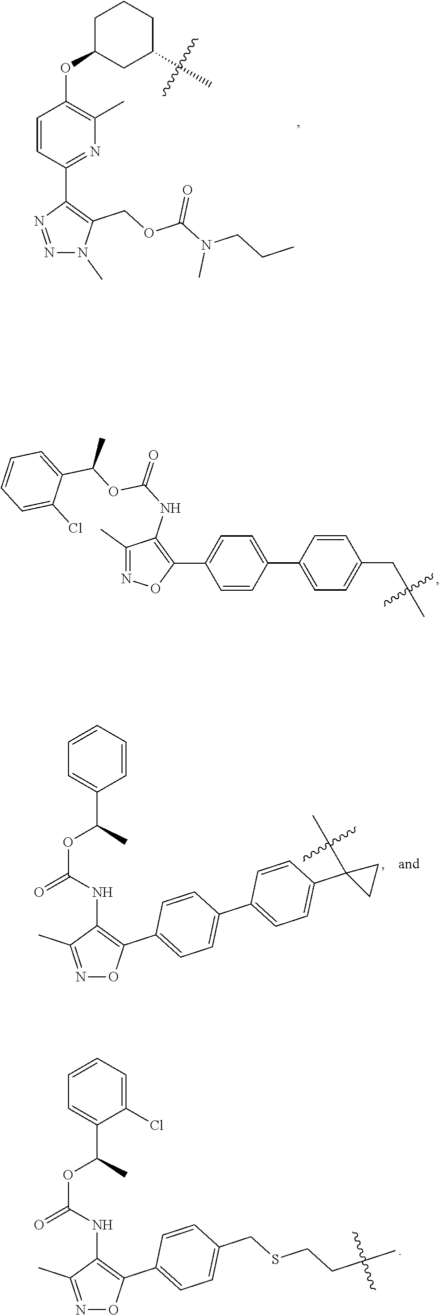

In some embodiments is a compound of Formula (III), wherein R² is

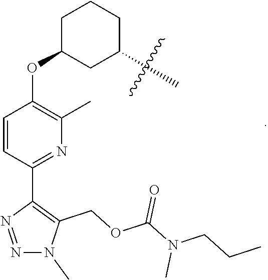 [SMILES: CCCN(C)C(=O)OCc1c(-c2ccc(O[C@H]3CCC[C@H](C)C3)c(C)n2)nnn1C]

In some embodiments is a compound of Formula (III), wherein R² is

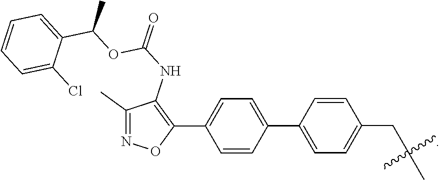 [SMILES: *Cc1ccc(-c2ccc(-c3onc(C)c3NC(=O)O[C@H](C)c3ccccc3Cl)cc2)cc1]

In some embodiments is a compound of Formula (III), wherein R² is

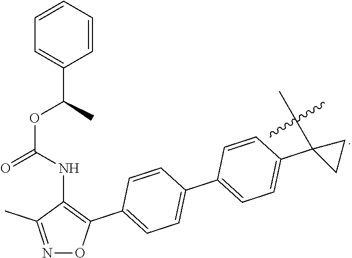 [SMILES: *C1(c2ccc(-c3ccc(-c4onc(C)c4NC(=O)O[C@H](C)c4ccccc4)cc3)cc2)CC1]

In some embodiments is a compound of Formula (III), wherein R² is

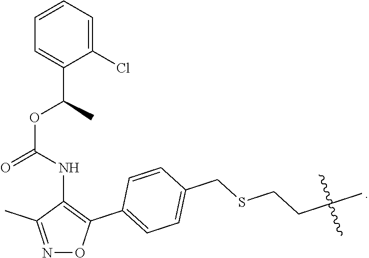 [SMILES: *CCSCc1ccc(-c2onc(C)c2NC(=O)O[C@H](C)c2ccccc2Cl)cc1]

In some embodiments is a compound of Formula (III), wherein R¹ is optionally substituted C₁₋₆alkyl. In some embodiments is a compound of Formula (III), wherein R¹ is C₁₋₆ alkyl optionally substituted with 1, 2, 3, 4, or 5 groups independently selected from halogen, —CN, —OH, —NH₂, —N(H)(C₁₋₆alkyl), N(C₁₋₆alkyl)₂, —C(O)OH, —C(O)O—C₁₋₆alkyl, —C(O)NH₂, —C(O)N(H)(C₁₋₆alkyl), —C(O)N(C₁₋₆alkyl)₂, —C(O)C₁₋₆alkyl, —S(O)₂C₁₋₆alkyl, —S(O)₂NH₂, —S(O)₂N(H)(C₁₋₆alkyl), —S(O)₂N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments is a compound of Formula (III), wherein R¹ is C₁₋₆alkyl optionally substituted with 1, 2, 3, 4, or 5 groups independently selected from halogen, —OH, —NH₂, —N(H)(C₁₋₆alkyl), N(C₁₋₆alkyl)₂, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments is a compound of Formula (III), wherein R¹ is C₁₋₆alkyl optionally substituted with 1, 2, 3, 4, or 5 groups independently selected from halogen, —OH, —NH₂, —N(H)(C₁₋₆alkyl), N(C₁₋₆alkyl)₂, C₁₋₆haloalkyl, and C₁₋₆alkoxy. In some embodiments is a compound of Formula (III), wherein R¹ is unsubstituted C₁₋₆alkyl.

In some embodiments is a compound of Formula (III), wherein R¹ is optionally substituted C₃₋₆cycloalkyl. In some embodiments is a compound of Formula (III), wherein R¹ is C₃₋₆cycloalkyl optionally substituted with 1, 2, 3, 4, or 5 groups independently selected from halogen, —CN, —OH, —NH₂, —N(H)(C₁₋₆alkyl), N(C₁₋₆alkyl)₂, —C(O)OH, —C(O)O—C₁₋₆alkyl, —C(O)NH₂, —C(O)N(H)(C₁₋₆alkyl), —C(O)N(C₁₋₆alkyl)₂, —C(O)C₁₋₆alkyl, —S(O)₂C₁₋₆alkyl, —S(O)₂NH₂, —S(O)₂N(H)(C₁₋₆alkyl), —S(O)₂N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments is a compound of Formula (III), wherein R¹ is C₃₋₆cycloalkyl optionally substituted with 1, 2, 3, 4, or 5 groups independently selected from halogen, —OH, —NH₂, —N(H)(C₁₋₆alkyl), N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉ heteroaryl. In some embodiments is a compound of Formula (III), wherein R¹ is C₃₋₆cycloalkyl optionally substituted with 1, 2, 3, 4, or 5 groups independently selected from halogen, —OH, —NH₂, —N(H)(C₁₋₆alkyl), N(C₁₋₆alkyl)₂, C₁₋₆alkyl, C₁₋₆haloalkyl, and C₁₋₆alkoxy. In some embodiments is a compound of Formula (III), wherein R¹ is unsubstituted C₃₋₆cycloalkyl.

In some embodiments is a compound of Formula (III), wherein R¹ is optionally substituted C₂₋₉heterocycloalkyl. In some embodiments is a compound of Formula (III), wherein R¹ is C₂₋₉heterocycloalkyl optionally substituted with 1, 2, 3, 4, or 5 groups independently selected from halogen, —CN, —OH, —NH₂, —N(H)(C₁₋₆alkyl), N(C₁₋₆alkyl)₂, —C(O)OH, —C(O)O—C₁₋₆alkyl, —C(O)NH₂, —C(O)N(H)(C₁₋₆alkyl), —C(O)N(C₁₋₆alkyl)₂, —C(O)C₁₋₆alkyl, —S(O)₂C₁₋₆alkyl, —S(O)₂NH₂, —S(O)₂N(H)(C₁₋₆alkyl), —S(O)₂N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments is a compound of Formula (III), wherein R¹ is C₂₋₉heterocycloalkyl optionally substituted with 1, 2, 3, 4, or 5 groups independently selected from halogen, —OH, —NH₂, —N(H)(C₁₋₆alkyl), N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments is a compound of Formula (III), wherein R¹ is C₂₋₉heterocycloalkyl optionally substituted with 1, 2, 3, 4, or 5 groups independently selected from halogen, —OH, —NH₂, —N(H)(C₁₋₆alkyl), N(C₁₋₆alkyl)₂, C₁₋₆alkyl, C₁₋₆haloalkyl, and C₁₋₆alkoxy. In some embodiments is a compound of Formula (III), wherein R¹ is unsubstituted C₂₋₉heterocycloalkyl.

In some embodiments is a compound of Formula (III), wherein R¹ is optionally substituted C₆₋₁₀aryl. In some embodiments is a compound of Formula (III), wherein R¹ is C₆₋₁₀aryl optionally substituted with 1, 2, 3, 4, or 5 groups independently selected from halogen, —CN, —OH, —NH₂, —N(H)(C₁₋₆alkyl), N(C₁₋₆alkyl)₂, —C(O)OH, —C(O)O—C₁₋₆alkyl, —C(O)NH₂, —C(O)N(H)(C₁₋₆alkyl), —C(O)N(C₁₋₆alkyl)₂, —C(O)C₁₋₆alkyl, —S(O)₂C₁₋₆alkyl, —S(O)₂NH₂, —S(O)₂N(H)(C₁₋₆alkyl), —S(O)₂N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments is a compound of Formula (III), wherein R¹ is C₆₋₁₀aryl optionally substituted with 1, 2, 3, 4, or 5 groups independently selected from halogen, —OH, —NH₂, —N(H)(C₁₋₆alkyl), N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments is a compound of Formula (III), wherein R¹ is C₆₋₁₀aryl optionally substituted with 1, 2, 3, 4, or 5 groups independently selected from halogen, —OH, —NH₂, —N(H)(C₁₋₆alkyl), N(C₁₋₆alkyl)₂, C₁₋₆alkyl, C₁₋₆haloalkyl, and C₁₋₆alkoxy. In some embodiments is a compound of Formula (III), wherein R¹ is unsubstituted C₆₋₁₀aryl.

In some embodiments is a compound of Formula (III), wherein R¹ is optionally substituted C₁₋₉heteroaryl. In some embodiments is a compound of Formula (III), wherein R¹ is C₁₋₉heteroaryl optionally substituted with 1, 2, 3, 4, or 5 groups independently selected from halogen, —CN, —OH, —NH₂, —N(H)(C₁₋₆alkyl), N(C₁₋₆alkyl)₂, —C(O)OH, —C(O)O—C₁₋₆alkyl, —C(O)NH₂, —C(O)N(H)(C₁₋₆alkyl), —C(O)N(C₁₋₆alkyl)₂, —C(O)C₁₋₆alkyl, —S(O)₂C₁₋₆alkyl, —S(O)₂NH₂, —S(O)₂N(H)(C₁₋₆alkyl), —S(O)₂N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments is a compound of Formula (III), wherein R¹ is C₁₋₉heteroaryl optionally substituted with 1, 2, 3, 4, or 5 groups independently selected from halogen, —OH, —NH₂, —N(H)(C₁₋₆alkyl), N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉ heteroaryl. In some embodiments is a compound of Formula (III), wherein R¹ is C₁₋₉heteroaryl optionally substituted with 1, 2, 3, 4, or 5 groups independently selected from halogen, —OH, —NH₂, —N(H)(C₁₋₆alkyl), N(C₁₋₆alkyl)₂, C₁₋₆alkyl, C₁₋₆haloalkyl, and C₁₋₆alkoxy. In some embodiments is a compound of Formula (III), wherein R¹ is unsubstituted C₁₋₉heteroaryl.

In some embodiments is a compound of Formula (III), wherein R¹ is —CH₃. In some embodiments is a compound of Formula (III), wherein R¹ is —CH₂CH₃. In some embodiments is a compound of Formula (III), wherein R¹ is —CH₂CH₂CH₃. In some embodiments is a compound of Formula (III), wherein R¹ is —CHCH₃)₂. In some embodiments is a compound of Formula (III), wherein R² is

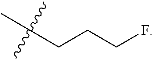 [SMILES: *CCCF]

In some embodiments is a compound of Formula (III), wherein R¹ is

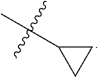 [SMILES: *C1CC1]

In some embodiments is a compound of Formula (III), wherein R¹ is

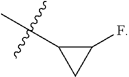 [SMILES: *C1CC1F]

In some embodiments is a compound of Formula (III), wherein R¹ is N

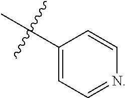 [SMILES: *c1ccncc1]

In some embodiments is a compound of Formula (III), wherein R¹ is

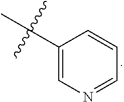 [SMILES: *c1cccnc1]

In some embodiments is a compound of Formula (III), wherein R¹ is

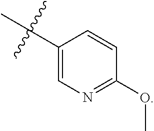 [SMILES: *c1ccc(OC)nc1]

In some embodiments is a compound of Formula (III), wherein R¹ is

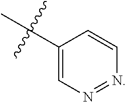 [SMILES: *c1ccnnc1]

In some embodiments is a compound of Formula (III), wherein R² is

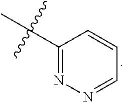 [SMILES: *c1cccnn1]

In some embodiments is a compound of Formula (III), wherein R¹ is

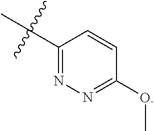 [SMILES: *c1ccc(OC)nn1]

In some embodiments is a compound of Formula (III wherein R¹ is

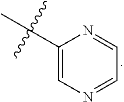 [SMILES: *c1cnccn1]

In some embodiments is a compound of Formula (III), wherein R¹ is

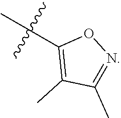 [SMILES: *c1onc(C)c1C]

In some embodiments the compound of Formula (III) is selected from:

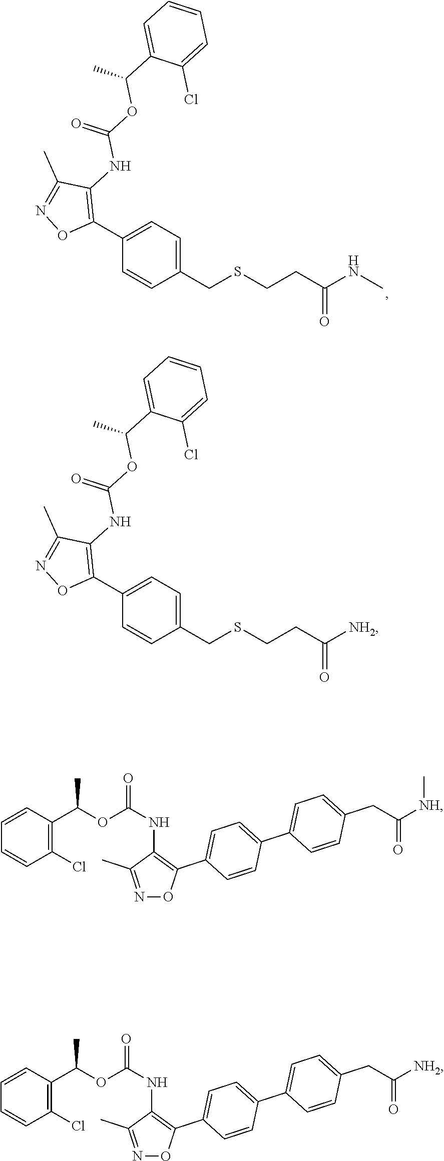 [SMILES: CNC(=O)CCSCc1ccc(-c2onc(C)c2NC(=O)O[C@H](C)c2ccccc2Cl)cc1 ; Cc1noc(-c2ccc(CSCCC(N)=O)cc2)c1NC(=O)O[C@H](C)c1ccccc1Cl ; CNC(=O)Cc1ccc(-c2ccc(-c3onc(C)c3NC(=O)O[C@H](C)c3ccccc3Cl)cc2)cc1 ; Cc1noc(-c2ccc(-c3ccc(CC(N)=O)cc3)cc2)c1NC(=O)O[C@H](C)c1ccccc1Cl]

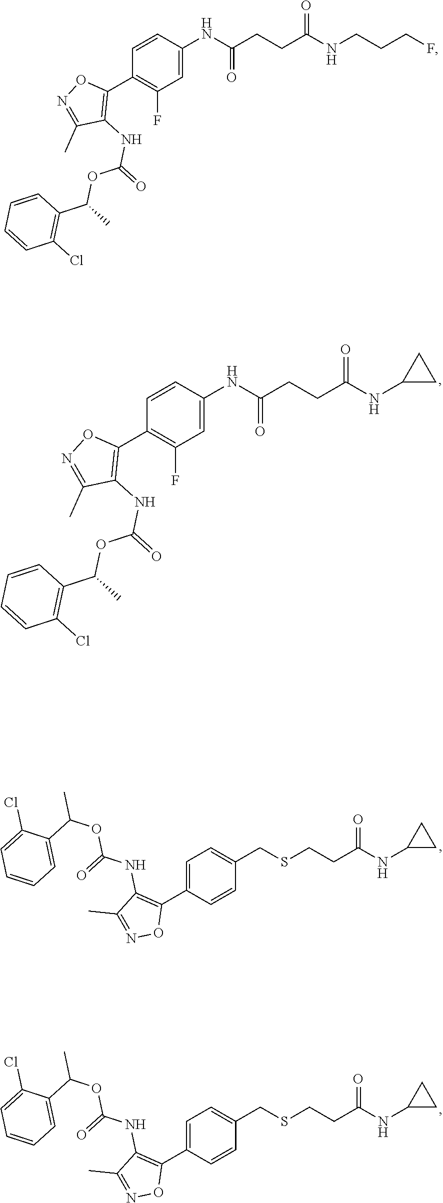 [SMILES: Cc1noc(-c2ccc(NC(=O)CCC(=O)NCCCF)cc2F)c1NC(=O)O[C@H](C)c1ccccc1Cl ; Cc1noc(-c2ccc(NC(=O)CCC(=O)NC3CC3)cc2F)c1NC(=O)O[C@H](C)c1ccccc1Cl ; Cc1noc(-c2ccc(CSCCC(=O)NC3CC3)cc2)c1NC(=O)OC(C)c1ccccc1Cl ; Cc1noc(-c2ccc(CSCCC(=O)NC3CC3)cc2)c1NC(=O)OC(C)c1ccccc1Cl]

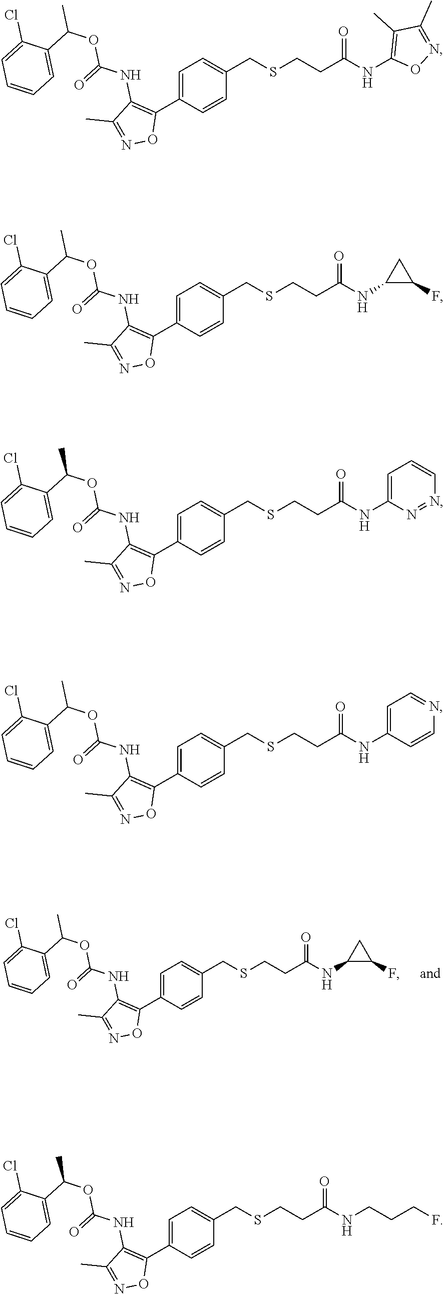 [SMILES: Cc1noc(NC(=O)CCSCc2ccc(-c3onc(C)c3NC(=O)OC(C)c3ccccc3Cl)cc2)c1C ; Cc1noc(-c2ccc(CSCCC(=O)N[C@@H]3C[C@H]3F)cc2)c1NC(=O)OC(C)c1ccccc1Cl ; Cc1noc(-c2ccc(CSCCC(=O)Nc3cccnn3)cc2)c1NC(=O)O[C@H](C)c1ccccc1Cl ; Cc1noc(-c2ccc(CSCCC(=O)Nc3ccncc3)cc2)c1NC(=O)OC(C)c1ccccc1Cl ; Cc1noc(-c2ccc(CSCCC(=O)N[C@H]3C[C@H]3F)cc2)c1NC(=O)OC(C)c1ccccc1Cl ; Cc1noc(-c2ccc(CSCCC(=O)NCCCF)cc2)c1NC(=O)O[C@H](C)c1ccccc1Cl]

In some embodiments described herein is a compound, or a pharmaceutically acceptable salt or solvate thereof, of Formula (IV):

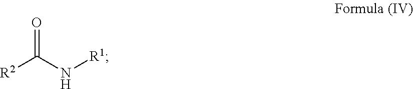

wherein:
R¹ is an amide prodrug moiety, wherein the amide prodrug moiety is enzymatically cleaved by fatty acid amide hydrolase (FAAH) in the brain to release

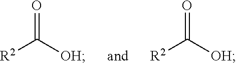

is a moiety that modulates GPR120 in the brain.

In some embodiments is a compound of Formula (IV), wherein R² is

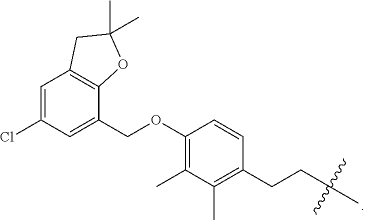 [SMILES: *CCc1ccc(OCc2cc(Cl)cc3c2OC(C)(C)C3)c(C)c1C]

In some embodiments is a compound of Formula (IV), wherein R¹ is optionally substituted C₁₋₆alkyl. In some embodiments is a compound of Formula (IV), wherein R¹ is C₁₋₆ alkyl optionally substituted with 1, 2, 3, 4, or 5 groups independently selected from halogen, —CN, —OH, —NH₂, —N(H)(C₁₋₆alkyl), N(C₁₋₆alkyl)₂, —C(O)OH, —C(O)O—C₁₋₆alkyl, —C(O)NH₂, —C(O)N(H)(C₁₋₆alkyl), —C(O)N(C₁₋₆alkyl)₂, —C(O)C₁₋₆alkyl, —S(O)₂C₁₋₆alkyl, —S(O)₂NH₂, —S(O)₂N(H)(C₁₋₆alkyl), —S(O)₂N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments is a compound of Formula (IV), wherein R¹ is C₁₋₆alkyl optionally substituted with 1, 2, 3, 4, or 5 groups independently selected from halogen, —OH, —NH₂, —N(H)(C₁₋₆alkyl), N(C₁₋₆alkyl)₂, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments is a compound of Formula (IV), wherein R¹ is C₁₋₆alkyl optionally substituted with 1, 2, 3, 4, or 5 groups independently selected from halogen, —OH, —NH₂, —N(H)(C₁₋₆alkyl), N(C₁₋₆alkyl)₂, C₁₋₆haloalkyl, and C₁₋₆alkoxy. In some embodiments is a compound of Formula (IV), wherein R¹ is unsubstituted C₁₋₆alkyl.

In some embodiments is a compound of Formula (IV), wherein R¹ is optionally substituted C₃₋₆cycloalkyl. In some embodiments is a compound of Formula (IV), wherein R¹ is C₃₋₆cycloalkyl optionally substituted with 1, 2, 3, 4, or 5 groups independently selected from halogen, —CN, —OH, —NH₂, —N(H)(C₁₋₆alkyl), N(C₁₋₆alkyl)₂, —C(O)OH, —C(O)O—C₁₋₆alkyl, —C(O)NH₂, —C(O)N(H)(C₁₋₆alkyl), —C(O)N(C₁₋₆alkyl)₂, —C(O)C₁₋₆alkyl, —S(O)₂C₁₋₆alkyl, —S(O)₂NH₂, —S(O)₂N(H)(C₁₋₆alkyl), —S(O)₂N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments is a compound of Formula (IV), wherein R¹ is C₃₋₆cycloalkyl optionally substituted with 1, 2, 3, 4, or 5 groups independently selected from halogen, —OH, —NH₂, —N(H)(C₁₋₆alkyl), N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉ heteroaryl. In some embodiments is a compound of Formula (IV), wherein R¹ is C₃₋₆cycloalkyl optionally substituted with 1, 2, 3, 4, or 5 groups independently selected from halogen, —OH, —NH₂, —N(H)(C₁₋₆alkyl), N(C₁₋₆alkyl)₂, C₁₋₆alkyl, C₁₋₆haloalkyl, and C₁₋₆alkoxy. In some embodiments is a compound of Formula (IV), wherein R¹ is unsubstituted C₃₋₆cycloalkyl.

In some embodiments is a compound of Formula (IV), wherein R¹ is optionally substituted C₂₋₉heterocycloalkyl. In some embodiments is a compound of Formula (IV), wherein R¹ is C₂₋₉heterocycloalkyl optionally substituted with 1, 2, 3, 4, or 5 groups independently selected from halogen, —CN, —OH, —NH₂, —N(H)(C₁₋₆alkyl), N(C₁₋₆alkyl)₂, —C(O)OH, —C(O)O—C₁₋₆alkyl, —C(O)NH₂, —C(O)N(H)(C₁₋₆alkyl), —C(O)N(C₁₋₆alkyl)₂, —C(O)C₁₋₆alkyl, —S(O)₂C₁₋₆alkyl, —S(O)₂NH₂, —S(O)₂N(H)(C₁₋₆alkyl), —S(O)₂N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments is a compound of Formula (IV), wherein R¹ is C₂₋₉heterocycloalkyl optionally substituted with 1, 2, 3, 4, or 5 groups independently selected from halogen, —OH, —NH₂, —N(H)(C₁₋₆alkyl), N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments is a compound of Formula (IV), wherein R¹ is C₂₋₉heterocycloalkyl optionally substituted with 1, 2, 3, 4, or 5 groups independently selected from halogen, —OH, —NH₂, —N(H)(C₁₋₆alkyl), N(C₁₋₆alkyl)₂, C₁₋₆alkyl, C₁₋₆haloalkyl, and C₁₋₆alkoxy. In some embodiments is a compound of Formula (IV), wherein R¹ is unsubstituted C₂₋₉heterocycloalkyl.

In some embodiments is a compound of Formula (IV), wherein R¹ is optionally substituted C₆₋₁₀aryl. In some embodiments is a compound of Formula (IV), wherein R¹ is C₆₋₁₀aryl optionally substituted with 1, 2, 3, 4, or 5 groups independently selected from halogen, —CN, —OH, —NH₂, —N(H)(C₁₋₆alkyl), N(C₁₋₆alkyl)₂, —C(O)OH, —C(O)O—C₁₋₆alkyl, —C(O)NH₂, —C(O)N(H)(C₁₋₆alkyl), —C(O)N(C₁₋₆alkyl)₂, —C(O)C₁₋₆alkyl, —S(O)₂C₁₋₆alkyl, —S(O)₂NH₂, —S(O)₂N(H)(C₁₋₆alkyl), —S(O)₂N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments is a compound of Formula (IV), wherein R¹ is C₆₋₁₀aryl optionally substituted with 1, 2, 3, 4, or 5 groups independently selected from halogen, —OH, —NH₂, —N(H)(C₁₋₆alkyl), N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments is a compound of Formula (IV), wherein R¹ is C₆₋₁₀aryl optionally substituted with 1, 2, 3, 4, or 5 groups independently selected from halogen, —OH, —NH₂, —N(H)(C₁₋₆alkyl), N(C₁₋₆alkyl)₂, C₁₋₆alkyl, C₁₋₆haloalkyl, and C₁₋₆alkoxy. In some embodiments is a compound of Formula (IV), wherein R¹ is unsubstituted C₆₋₁₀aryl.

In some embodiments is a compound of Formula (IV), wherein R¹ is optionally substituted C₁₋₉heteroaryl. In some embodiments is a compound of Formula (IV), wherein R¹ is C₁₋₉heteroaryl optionally substituted with 1, 2, 3, 4, or 5 groups independently selected from halogen, —CN, —OH, —NH₂, —N(H)(C₁₋₆alkyl), N(C₁₋₆alkyl)₂, —C(O)OH, —C(O)O—C₁₋₆alkyl, —C(O)NH₂, —C(O)N(H)(C₁₋₆alkyl), —C(O)N(C₁₋₆alkyl)₂, —C(O)C₁₋₆alkyl, —S(O)₂C₁₋₆alkyl, —S(O)₂NH₂, —S(O)₂N(H)(C₁₋₆alkyl), —S(O)₂N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments is a compound of Formula (IV), wherein R¹ is C₁₋₉heteroaryl optionally substituted with 1, 2, 3, 4, or 5 groups independently selected from halogen, —OH, —NH₂, —N(H)(C₁₋₆alkyl), N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉ heteroaryl. In some embodiments is a compound of Formula (IV), wherein R¹ is C₁₋₉heteroaryl optionally substituted with 1, 2, 3, 4, or 5 groups independently selected from halogen, —OH, —NH₂, —N(H)(C₁₋₆alkyl), N(C₁₋₆alkyl)₂, C₁₋₆alkyl, C₁₋₆haloalkyl, and C₁₋₆alkoxy. In some embodiments is a compound of Formula (IV), wherein R¹ is unsubstituted C₁₋₉heteroaryl.

In some embodiments is a compound of Formula (IV), wherein R¹ is —CH₃. In some embodiments is a compound of Formula (IV), wherein R¹ is —CH₂CH₃. In some embodiments is a compound of Formula (IV), wherein R¹ is —CH₂CH₂CH₃. In some embodiments is a compound of Formula (IV), wherein R¹ is —CHCH₃)₂. In some embodiments is a compound of Formula (IV), wherein R² is

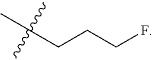 [SMILES: *CCCF]

In some embodiments is a compound of Formula (IV), wherein R¹ is

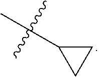 [SMILES: *C1CC1]

In some embodiments is a compound of Formula (IV), wherein R¹ is

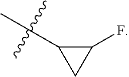 [SMILES: *C1CC1F]

In some embodiments is a compound of Formula (IV) wherein R¹ is

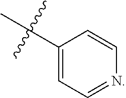 [SMILES: *c1ccncc1]

In some embodiments is a compound of Formula (IV), wherein R¹ is

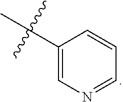 [SMILES: *c1cccnc1]

In some embodiments is a compound of Formula (IV), wherein R¹ is

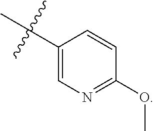 [SMILES: *c1ccc(OC)nc1]

In some embodiments is a compound of Formula (IV), wherein R¹ is

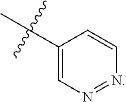 [SMILES: *c1ccnnc1]

In some embodiments is a compound of Formula (IV), wherein R¹ is

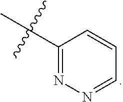 [SMILES: *c1cccnn1]

In some embodiments is a compound of Formula (IV), wherein R¹ is

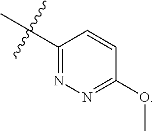 [SMILES: *c1ccc(OC)nn1]

In some embodiments is a compound of Formula (IV), wherein R¹ is

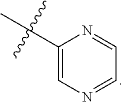 [SMILES: *c1cnccn1]

In some embodiments is a compound of Formula (IV), wherein R¹ is

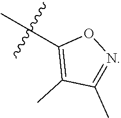 [SMILES: *c1onc(C)c1C]

In some embodiments the compound of Formula (IV) is selected from:

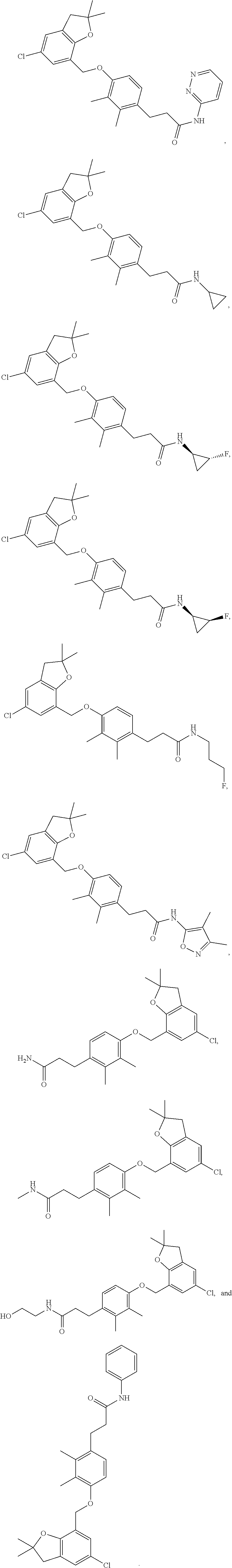

In some embodiments described herein is a compound, or a pharmaceutically acceptable salt or solvate thereof, of Formula (V):

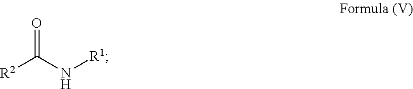

wherein:
R¹ is an amide prodrug moiety, wherein the amide prodrug moiety is enzymatically cleaved by fatty acid amide hydrolase (FAAH) in the brain to release

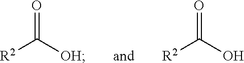

is a moiety that modulates TTR in the brain.

In some embodiments is a compound of Formula (V), wherein R² is

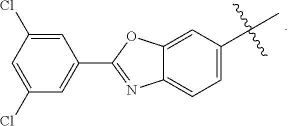 [SMILES: *c1ccc2nc(-c3cc(Cl)cc(Cl)c3)oc2c1]

In some embodiments is a compound of Formula (V), wherein R² is

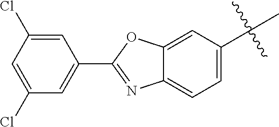 [SMILES: *c1ccc2nc(-c3cc(Cl)cc(Cl)c3)oc2c1]

and R¹ is C₁₋₄alkyl. In some embodiments is a compound of Formula (V), wherein R² is

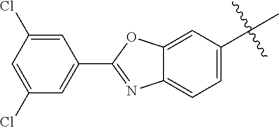 [SMILES: *c1ccc2nc(-c3cc(Cl)cc(Cl)c3)oc2c1]

and R¹ is C₁₋₄haloalkyl.

In some embodiments is a compound of Formula (V), wherein R² is

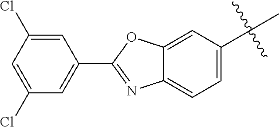 [SMILES: *c1ccc2nc(-c3cc(Cl)cc(Cl)c3)oc2c1]

and R¹ is optionally substituted C₃₋₆cycloalkyl. In some embodiments is a compound of Formula (V), wherein R² is

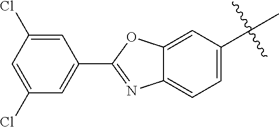 [SMILES: *c1ccc2nc(-c3cc(Cl)cc(Cl)c3)oc2c1]

and R¹ is C₃₋₆cycloalkyl optionally substituted with 1, 2, 3, 4, or 5 groups independently selected from halogen, —CN, —OH, —NH₂, —N(H)(C₁₋₆alkyl), N(C₁₋₆alkyl)₂, —C(O)OH, —C(O)O—C₁₋₆alkyl, —C(O)NH₂, —C(O)N(H)(C₁₋₆alkyl), —C(O)N(C₁₋₆alkyl)₂, —C(O)C₁₋₆alkyl, —S(O)₂C₁₋₆alkyl, —S(O)₂NH₂, —S(O)₂N(H)(C₁₋₆alkyl), —S(O)₂N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments is a compound of Formula (V), wherein R² is

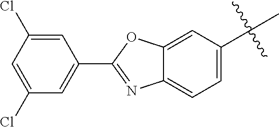 [SMILES: *c1ccc2nc(-c3cc(Cl)cc(Cl)c3)oc2c1]

and R¹ is C₃₋₈cycloalkyl optionally substituted with 1, 2, 3, 4, or 5 groups independently selected from halogen, —OH, —NH₂, —N(H)(C₁₋₆alkyl), N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments is a compound of Formula (V), wherein R² is

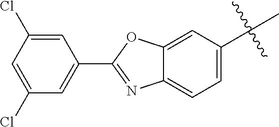 [SMILES: *c1ccc2nc(-c3cc(Cl)cc(Cl)c3)oc2c1]

and R¹ is C₃₋₆cycloalkyl optionally substituted with 1, 2, 3, 4, or 5 groups independently selected from halogen, —OH, —NH₂, —N(H)(C₁₋₆alkyl), N(C₁₋₆alkyl)₂, C₁₋₆alkyl, C₁₋₆haloalkyl, and C₁₋₆alkoxy. In some embodiments is a compound of Formula (V), wherein R² is

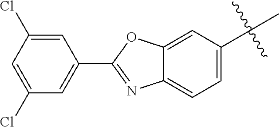 [SMILES: *c1ccc2nc(-c3cc(Cl)cc(Cl)c3)oc2c1]

and R¹ is unsubstituted C₃₋₆cycloalkyl.

In some embodiments is a compound of Formula (V), wherein R² is

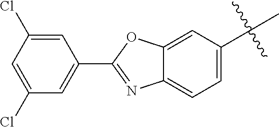 [SMILES: *c1ccc2nc(-c3cc(Cl)cc(Cl)c3)oc2c1]

and R¹ is optionally substituted C₆₋₁₀aryl. In some embodiments is a compound of Formula (V), wherein R² is

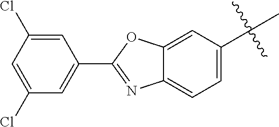 [SMILES: *c1ccc2nc(-c3cc(Cl)cc(Cl)c3)oc2c1]

and R¹ is C₆₋₁₀aryl optionally substituted with 1, 2, 3, 4, or 5 groups independently selected from halogen, —CN, —OH, —NH₂, —N(H)(C₁₋₆alkyl), N(C₁₋₆alkyl)₂, —C(O)OH, —C(O)O—C₁₋₆alkyl, —C(O)NH₂, —C(O)N(H)(C₁₋₆alkyl), —C(O)N(C₁₋₆alkyl)₂, —C(O)C₁₋₆alkyl, —S(O)₂C₁₋₆alkyl, —S(O)₂NH₂, —S(O)₂N(H)(C₁₋₆alkyl), —S(O)₂N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments is a compound of Formula (V), wherein R² is

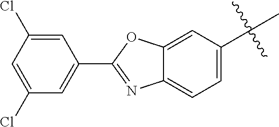 [SMILES: *c1ccc2nc(-c3cc(Cl)cc(Cl)c3)oc2c1]

and R¹ is C₆₋₁₀aryl optionally substituted with 1, 2, 3, 4, or 5 groups independently selected from halogen, —OH, —NH₂, —N(H)(C₁₋₆alkyl), N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments is a compound of Formula (V), wherein R² is

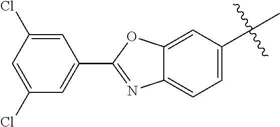 [SMILES: *c1ccc2nc(-c3cc(Cl)cc(Cl)c3)oc2c1]

and R¹ is C₆₋₁₀aryl optionally substituted with 1, 2, 3, 4, or 5 groups independently selected from halogen, —OH, —NH₂, —N(H)(C₁₋₆alkyl), N(C₁₋₆alkyl)₂, C₁₋₆alkyl, C₁₋₆haloalkyl, and C₁₋₆alkoxy. In some embodiments is a compound of Formula (V), wherein R² is

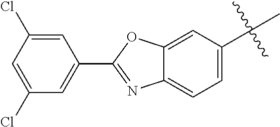 [SMILES: *c1ccc2nc(-c3cc(Cl)cc(Cl)c3)oc2c1]

and R¹ is unsubstituted C₆₋₁₀aryl.

In some embodiments is a compound of Formula (V), wherein R² is

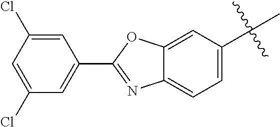 [SMILES: *c1ccc2nc(-c3cc(Cl)cc(Cl)c3)oc2c1]

and R¹ is optionally substituted C₁₋₉heteroaryl. In some embodiments is a compound of Formula (V), wherein R² is

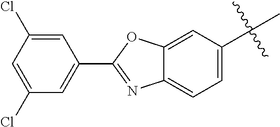 [SMILES: *c1ccc2nc(-c3cc(Cl)cc(Cl)c3)oc2c1]

and R¹ is C₁₋₉heteroaryl optionally substituted with 1, 2, 3, 4, or 5 groups independently selected from halogen, —CN, —OH, —NH₂, —N(H)(C₁₋₆alkyl), N(C₁₋₆alkyl)₂, —C(O)OH, —C(O)O—C₁₋₆alkyl, —C(O)NH₂, —C(O)N(H)(C₁₋₆alkyl), —C(O)N(C₁₋₆alkyl)₂, —C(O)C₁₋₆alkyl, —S(O)₂C₁₋₆alkyl, —S(O)₂NH₂, —S(O)₂N(H)(C₁₋₆alkyl), —S(O)₂N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments is a compound of Formula (V), wherein R² is

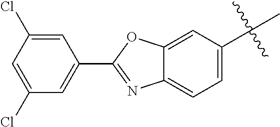 [SMILES: *c1ccc2nc(-c3cc(Cl)cc(Cl)c3)oc2c1]

and R¹ is C₁₋₉heteroaryl optionally substituted with 1, 2, 3, 4, or 5 groups independently selected from halogen, —OH, —NH₂, —N(H)(C₁₋₆alkyl), N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments is a compound of Formula (V), wherein R² is

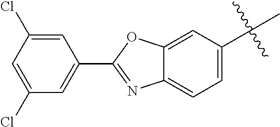 [SMILES: *c1ccc2nc(-c3cc(Cl)cc(Cl)c3)oc2c1]

and R¹ is C₁₋₉heteroaryl optionally substituted with 1, 2, 3, 4, or 5 groups independently selected from halogen, —OH, —NH₂, —N(H)(C₁₋₆alkyl), N(C₁₋₆alkyl)₂, C₁₋₆alkyl, C₁₋₆haloalkyl, and C₁₋₆alkoxy. In some embodiments is a compound of Formula (V), wherein R² is

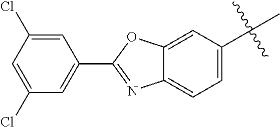 [SMILES: *c1ccc2nc(-c3cc(Cl)cc(Cl)c3)oc2c1]

and R¹ is unsubstituted C₁₋₉heteroaryl.

In some embodiments is a compound of Formula (V), wherein R² is

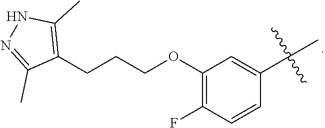 [SMILES: *c1ccc(F)c(OCCCc2c(C)n[nH]c2C)c1]

In some embodiments is a compound of Formula (V), wherein R² is

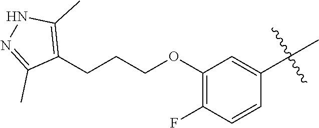 [SMILES: *c1ccc(F)c(OCCCc2c(C)n[nH]c2C)c1]

and R¹ is C₁₋₄alkyl. In some embodiments is a compound of Formula (V), wherein R² is

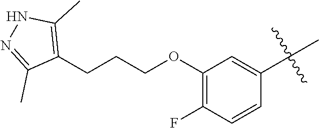 [SMILES: *c1ccc(F)c(OCCCc2c(C)n[nH]c2C)c1]

and R¹ is C₁₋₄haloalkyl.

In some embodiments is a compound of Formula (V), wherein R² is

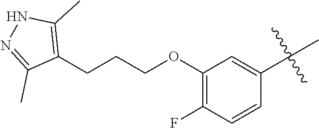 [SMILES: *c1ccc(F)c(OCCCc2c(C)n[nH]c2C)c1]

and R¹ is optionally substituted C₃₋₆cycloalkyl. In some embodiments is a compound of Formula (V), wherein R² is

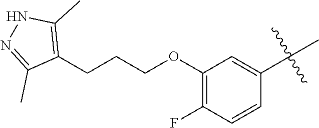 [SMILES: *c1ccc(F)c(OCCCc2c(C)n[nH]c2C)c1]

and R¹ is C₃₋₆cycloalkyl optionally substituted with 1, 2, 3, 4, or 5 groups independently selected from halogen, —CN, —OH, —NH₂, —N(H)(C₁₋₆alkyl), N(C₁₋₆alkyl)₂, —C(O)OH, —C(O)O—C₁₋₆alkyl, —C(O)NH₂, —C(O)N(H)(C₁₋₆alkyl), —C(O)N(C₁₋₆alkyl)₂, —C(O)C₁₋₆alkyl, —S(O)₂C₁₋₆alkyl, —S(O)₂NH₂, —S(O)₂N(H)(C₁₋₆alkyl), —S(O)₂N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments is a compound of Formula (V), wherein R² is

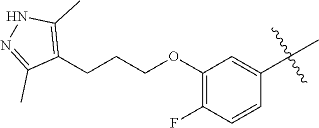 [SMILES: *c1ccc(F)c(OCCCc2c(C)n[nH]c2C)c1]

and R¹ is C₃₋₆cycloalkyl optionally substituted with 1, 2, 3, 4, or 5 groups independently selected from halogen, —OH, —NH₂, —N(H)(C₁₋₆alkyl), N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments is a compound of Formula (V), wherein R² is

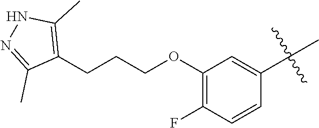 [SMILES: *c1ccc(F)c(OCCCc2c(C)n[nH]c2C)c1]

and R¹ is C₃₋₆cycloalkyl optionally substituted with 1, 2, 3, 4, or 5 groups independently selected from halogen, —OH, —NH₂, —N(H)(C₁₋₆alkyl), N(C₁₋₆alkyl)₂, C₁₋₆alkyl, C₁₋₆haloalkyl, and C₁₋₆alkoxy. In some embodiments is a compound of Formula (V), wherein R² is

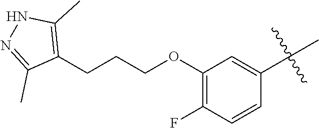 [SMILES: *c1ccc(F)c(OCCCc2c(C)n[nH]c2C)c1]

and R¹ is unsubstituted C₃₋₆cycloalkyl.

In some embodiments is a compound of Formula (V), wherein R² is

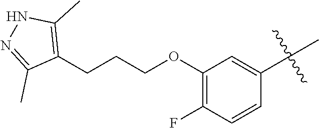 [SMILES: *c1ccc(F)c(OCCCc2c(C)n[nH]c2C)c1]

and R¹ is optionally substituted C₂₋₉heterocycloalkyl. In some embodiments is a compound of Formula (V), wherein R² is

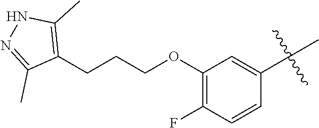 [SMILES: *c1ccc(F)c(OCCCc2c(C)n[nH]c2C)c1]

and R¹ is C₂₋₉heterocycloalkyl optionally substituted with 1, 2, 3, 4, or 5 groups independently selected from halogen, —CN, —OH, —NH₂, —N(H)(C₁₋₆alkyl), N(C₁₋₆alkyl)₂, —C(O)OH, —C(O)O—C₁₋₆alkyl, —C(O)NH₂, —C(O)N(H)(C₁₋₆alkyl), —C(O)N(C₁₋₆alkyl)₂, —C(O)C₁₋₆alkyl, —S(O)₂C₁₋₆alkyl, —S(O)₂NH₂, —S(O)₂N(H)(C₁₋₆alkyl), —S(O)₂N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments is a compound of Formula (V), wherein R² is

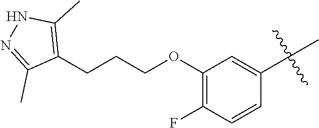 [SMILES: *c1ccc(F)c(OCCCc2c(C)n[nH]c2C)c1]

and R¹ is C₂₋₉heterocycloalkyl optionally substituted with 1, 2, 3, 4, or 5 groups independently selected from halogen, —OH, —NH₂, —N(H)(C₁₋₆alkyl), N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments is a compound of Formula (V), wherein R² is

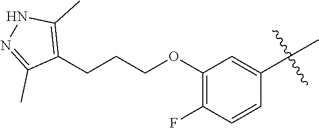 [SMILES: *c1ccc(F)c(OCCCc2c(C)n[nH]c2C)c1]

and R¹ is C₂₋₉heterocycloalkyl optionally substituted with 1, 2, 3, 4, or 5 groups independently selected from halogen, —OH, —NH₂, —N(H)(C₁₋₆alkyl), N(C₁₋₆alkyl)₂, C₁₋₆alkyl, C₁₋₆haloalkyl, and C₁₋₆alkoxy. In some embodiments is a compound of Formula (V), wherein R² is

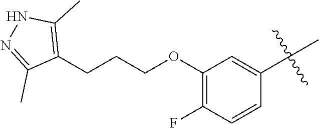 [SMILES: *c1ccc(F)c(OCCCc2c(C)n[nH]c2C)c1]

and R¹ is unsubstituted C₂₋₉heterocycloalkyl.

In some embodiments is a compound of Formula (V), wherein R² is

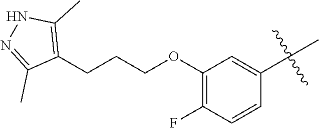 [SMILES: *c1ccc(F)c(OCCCc2c(C)n[nH]c2C)c1]

and R¹ is optionally substituted C₆₋₁₀aryl. In some embodiments is a compound of Formula (V), wherein R² is

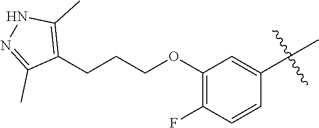 [SMILES: *c1ccc(F)c(OCCCc2c(C)n[nH]c2C)c1]

and R¹ is C₆₋₁₀aryl optionally substituted with 1, 2, 3, 4, or 5 groups independently selected from halogen, —CN, —OH, —NH₂, —N(H)(C₁₋₆alkyl), N(C₁₋₆alkyl)₂, —C(O)OH, —C(O)O—C₁₋₆alkyl, —C(O)NH₂, —C(O)N(H)(C₁₋₆alkyl), —C(O)N(C₁₋₆alkyl)₂, —C(O)C₁₋₆alkyl, —S(O)₂C₁₋₆alkyl, —S(O)₂NH₂, —S(O)₂N(H)(C₁₋₆alkyl), —S(O)₂N(C₁₋₆alkyl)₂, C₁₋₆alkyl, C₁₋₆haloalkyl, C₁₋₆alkoxy, C₃₋₈cycloalkyl, C₂₋₉heterocycloalkyl, C₆₋₁₀aryl, and C₁₋₉heteroaryl. In some embodiments is a compound of Formula (V), wherein R² is

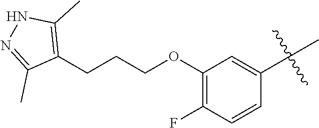 [SMILES: *c1ccc(F)c(OCCCc2c(C)n[nH]c2C)c1]

and R¹ is C₆₋₁₀aryl optionally substituted with 1, 2, 3, 4, or 5 groups independently selected from halogen, —OH, —NH₂, —N(H)(C₁₋₆alkyl), N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments is a compound of Formula (V), wherein R² is

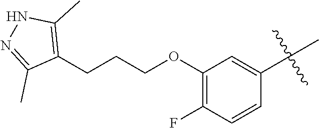 [SMILES: *c1ccc(F)c(OCCCc2c(C)n[nH]c2C)c1]

and R¹ is C₆₋₁₀aryl optionally substituted with 1, 2, 3, 4, or 5 groups independently selected from halogen, —OH, —NH₂, —N(H)(C₁₋₆alkyl), N(C₁₋₆alkyl)₂, C₁₋₆alkyl, C₁₋₆haloalkyl, and C₁₋₆alkoxy. In some embodiments is a compound of Formula (V), wherein R² is

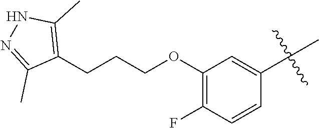 [SMILES: *c1ccc(F)c(OCCCc2c(C)n[nH]c2C)c1]

and R¹ is unsubstituted C₆₋₁₀aryl.

In some embodiments is a compound of Formula (V), wherein R² is

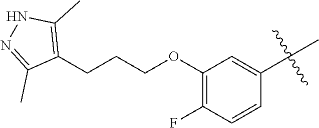 [SMILES: *c1ccc(F)c(OCCCc2c(C)n[nH]c2C)c1]

and R¹ is optionally substituted C₁₋₉heteroaryl. In some embodiments is a compound of Formula (V), wherein R² is

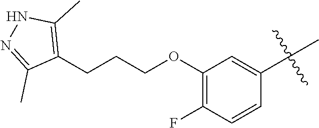 [SMILES: *c1ccc(F)c(OCCCc2c(C)n[nH]c2C)c1]

and R¹ is C₁₋₉heteroaryl optionally substituted with 1, 2, 3, 4, or 5 groups independently selected from halogen, —CN, —OH, —NH₂, —N(H)(C₁₋₆alkyl), N(C₁₋₆alkyl)₂, —C(O)OH, —C(O)O—C₁₋₆alkyl, —C(O)NH₂, —C(O)N(H)(C₁₋₆alkyl), —C(O)N(C₁₋₆alkyl)₂, —C(O)C₁₋₆alkyl, —S(O)₂C₁₋₆alkyl, —S(O)₂NH₂, —S(O)₂N(H)(C₁₋₆alkyl), —S(O)₂N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments is a compound of Formula (V), wherein R² is

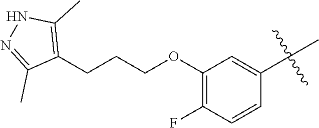 [SMILES: *c1ccc(F)c(OCCCc2c(C)n[nH]c2C)c1]

and R¹ is C₁₋₉heteroaryl optionally substituted with 1, 2, 3, 4, or 5 groups independently selected from halogen, —OH, —NH₂, —N(H)(C₁₋₆alkyl), N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments is a compound of Formula (V), wherein R² is

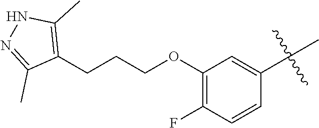 [SMILES: *c1ccc(F)c(OCCCc2c(C)n[nH]c2C)c1]

and R¹ is C₁₋₉heteroaryl optionally substituted with 1, 2, 3, 4, or 5 groups independently selected from halogen, —OH, —NH₂, —N(H)(C₁₋₆alkyl), N(C₁₋₆alkyl)₂, C₁₋₆alkyl, C₁₋₆haloalkyl, and C₁₋₆alkoxy. In some embodiments is a compound of Formula (V), wherein R² is

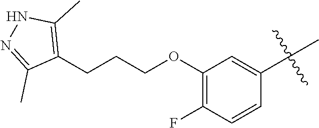 [SMILES: *c1ccc(F)c(OCCCc2c(C)n[nH]c2C)c1]

and R¹ is unsubstituted C₁₋₉heteroaryl.

In some embodiments is a compound of Formula (V), wherein R¹ is —CH₃. In some embodiments is a compound of Formula (V), wherein R¹ is —CH₂CH₃. In some embodiments is a compound of Formula (V), wherein R¹ is —CH₂CH₂CH₃. In some embodiments is a compound of Formula (V), wherein R¹ is —CHCH₃)₂. In some embodiments is a compound of Formula (V), wherein R¹ is

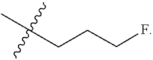 [SMILES: *CCCF]

In some embodiments is a compound of Formula (V), wherein R¹ is

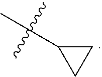 [SMILES: *C1CC1]

In some embodiments is a compound of Formula (V), wherein R¹ is

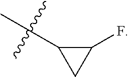 [SMILES: *C1CC1F]

In some embodiments is a compound of Formula (V), wherein R¹ is

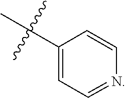 [SMILES: *c1ccncc1]

In some embodiments is a compound of Formula (V), wherein R¹ is

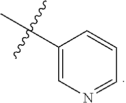 [SMILES: *c1cccnc1]

In some embodiments is a compound of Formula (V), wherein R¹ is

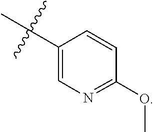 [SMILES: *c1ccc(OC)nc1]

In some embodiments is a compound of Formula (V), wherein R¹ is

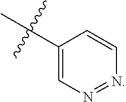 [SMILES: *c1ccnnc1]

In some embodiments is a compound of Formula (V), wherein R¹ is

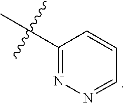 [SMILES: *c1cccnn1]

In some embodiments is a compound of Formula (V), wherein R¹ is

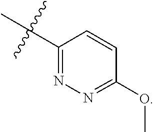 [SMILES: *c1ccc(OC)nn1]

In some embodiments is a compound of Formula (V), wherein R¹ is

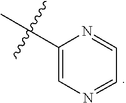 [SMILES: *c1cnccn1]

In some embodiments is a compound of Formula (V), wherein R¹ is

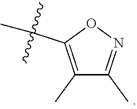 [SMILES: *c1onc(C)c1C]

In some embodiments the compound of Formula (V) is selected from:

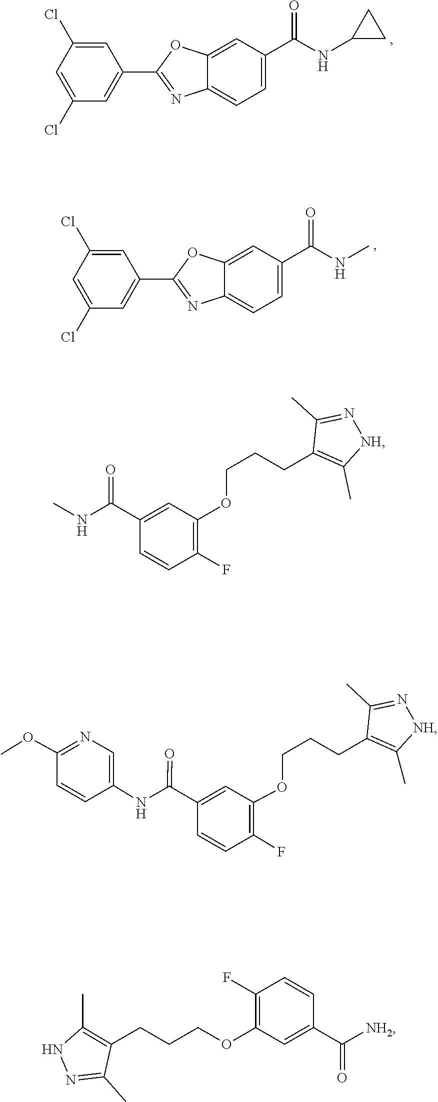

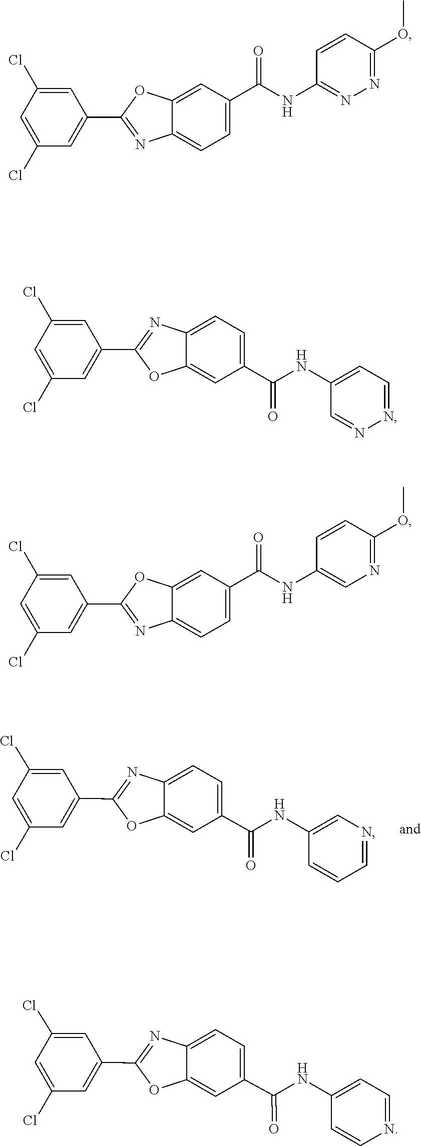 [SMILES: COc1ccc(NC(=O)c2ccc3nc(-c4cc(Cl)cc(Cl)c4)oc3c2)nn1 ; O=C(Nc1ccnnc1)c1ccc2nc(-c3cc(Cl)cc(Cl)c3)oc2c1 ; COc1ccc(NC(=O)c2ccc3nc(-c4cc(Cl)cc(Cl)c4)oc3c2)cn1 ; O=C(Nc1cccnc1)c1ccc2nc(-c3cc(Cl)cc(Cl)c3)oc2c1 ; O=C(Nc1ccncc1)c1ccc2nc(-c3cc(Cl)cc(Cl)c3)oc2c1]

In some embodiments described herein is a compound, or a pharmaceutically acceptable salt or solvate thereof, of Formula (VI):

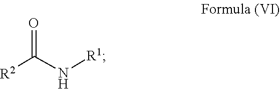

wherein:
R¹ is an amide prodrug moiety, wherein the amide prodrug moiety is enzymatically cleaved by fatty acid amide hydrolase (FAAH) in the brain to release

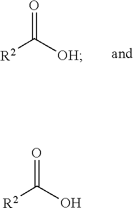

is a moiety that modulates PGI2 in the brain.

In some embodiments is a compound of Formula (VI), wherein R² is

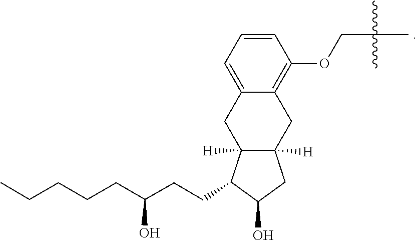

In some embodiments is a compound of Formula (VI), wherein R¹ is —CH₃.

In some embodiments is a compound of Formula (VI), wherein R¹ is optionally substituted C₃₋₆cycloalkyl. In some embodiments is a compound of Formula (VI), wherein R¹ is C₃₋₆cycloalkyl optionally substituted with 1, 2, 3, 4, or 5 groups independently selected from halogen, —CN, —OH, —NH₂, —N(H)(C₁₋₆alkyl), N(C₁₋₆alkyl)₂, —C(O)OH, —C(O)O—C₁₋₆alkyl, —C(O)NH₂, —C(O)N(H)(C₁₋₆alkyl), —C(O)N(C₁₋₆alkyl)₂, —C(O)C₁₋₆alkyl, —S(O)₂C₁₋₆alkyl, —S(O)₂NH₂, —S(O)₂N(H)(C₁₋₆alkyl), —S(O)₂N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments is a compound of Formula (VI), wherein R¹ is C₃₋₆cycloalkyl optionally substituted with 1, 2, 3, 4, or 5 groups independently selected from halogen, —OH, —NH₂, —N(H)(C₁₋₆alkyl), N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉ heteroaryl. In some embodiments is a compound of Formula (VI), wherein R¹ is C₃₋₆cycloalkyl optionally substituted with 1, 2, 3, 4, or 5 groups independently selected from halogen, —OH, —NH₂, —N(H)(C₁₋₆alkyl), N(C₁₋₆alkyl)₂, C₁₋₆alkyl, C₁₋₆haloalkyl, and C₁₋₆alkoxy. In some embodiments is a compound of Formula (VI), wherein R¹ is unsubstituted C₃₋₆cycloalkyl.

In some embodiments is a compound of Formula (VI), wherein R¹ is optionally substituted C₂₋₉heterocycloalkyl. In some embodiments is a compound of Formula (VI), wherein R¹ is C₂₋₉heterocycloalkyl optionally substituted with 1, 2, 3, 4, or 5 groups independently selected from halogen, —CN, —OH, —NH₂, —N(H)(C₁₋₆alkyl), N(C₁₋₆alkyl)₂, —C(O)OH, —C(O)O—C₁₋₆alkyl, —C(O)NH₂, —C(O)N(H)(C₁₋₆alkyl), —C(O)N(C₁₋₆alkyl)₂, —C(O)C₁₋₆alkyl, —S(O)₂C₁₋₆alkyl, —S(O)₂NH₂, —S(O)₂N(H)(C₁₋₆alkyl), —S(O)₂N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments is a compound of Formula (VI), wherein R¹ is C₂₋₉heterocycloalkyl optionally substituted with 1, 2, 3, 4, or 5 groups independently selected from halogen, —OH, —NH₂, —N(H)(C₁₋₆alkyl), N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments is a compound of Formula (VI), wherein R¹ is C₂₋₉heterocycloalkyl optionally substituted with 1, 2, 3, 4, or 5 groups independently selected from halogen, —OH, —NH₂, —N(H)(C₁₋₆alkyl), N(C₁₋₆alkyl)₂, C₁₋₆alkyl, C₁₋₆haloalkyl, and C₁₋₆alkoxy. In some embodiments is a compound of Formula (VI), wherein R¹ is unsubstituted C₂₋₉heterocycloalkyl.

In some embodiments is a compound of Formula (VI), wherein R¹ is optionally substituted C₆₋₁₀aryl. In some embodiments is a compound of Formula (VI), wherein R¹ is C₆₋₁₀aryl optionally substituted with 1, 2, 3, 4, or 5 groups independently selected from halogen, —CN, —OH, —NH₂, —N(H)(C₁₋₆alkyl), N(C₁₋₆alkyl)₂, —C(O)OH, —C(O)O—C₁₋₆alkyl, —C(O)NH₂, —C(O)N(H)(C₁₋₆alkyl), —C(O)N(C₁₋₆alkyl)₂, —C(O)C₁₋₆alkyl, —S(O)₂C₁₋₆alkyl, —S(O)₂NH₂, —S(O)₂N(H)(C₁₋₆alkyl), —S(O)₂N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments is a compound of Formula (VI), wherein R¹ is C₆₋₁₀aryl optionally substituted with 1, 2, 3, 4, or 5 groups independently selected from halogen, —OH, —NH₂, —N(H)(C₁₋₆alkyl), N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments is a compound of Formula (VI), wherein R¹ is C₆₋₁₀aryl optionally substituted with 1, 2, 3, 4, or 5 groups independently selected from halogen, —OH, —NH₂, —N(H)(C₁₋₆alkyl), N(C₁₋₆alkyl)₂, C₁₋₆alkyl, C₁₋₆haloalkyl, and C₁₋₆alkoxy. In some embodiments is a compound of Formula (VI), wherein R¹ is unsubstituted C₆₋₁₀aryl.

In some embodiments is a compound of Formula (VI), wherein R¹ is optionally substituted C₂₋₉heteroaryl. In some embodiments is a compound of Formula (VI), wherein R¹ is C₂₋₉heteroaryl optionally substituted with 1, 2, 3, 4, or 5 groups independently selected from halogen, —CN, —OH, —NH₂, —N(H)(C₁₋₆alkyl), N(C₁₋₆alkyl)₂, —C(O)OH, —C(O)O—C₁₋₆alkyl, —C(O)NH₂, —C(O)N(H)(C₁₋₆alkyl), —C(O)N(C₁₋₆alkyl)₂, —C(O)C₁₋₆alkyl, —S(O)₂C₁₋₆alkyl, —S(O)₂NH₂, —S(O)₂N(H)(C₁₋₆alkyl), —S(O)₂N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments is a compound of Formula (VI), wherein R¹ is C₂₋₉heteroaryl optionally substituted with 1, 2, 3, 4, or 5 groups independently selected from halogen, —OH, —NH₂, —N(H)(C₁₋₆alkyl), N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉ heteroaryl. In some embodiments is a compound of Formula (VI), wherein R¹ is C₂₋₉heteroaryl optionally substituted with 1, 2, 3, 4, or 5 groups independently selected from halogen, —OH, —NH₂, —N(H)(C₁₋₆alkyl), N(C₁₋₆alkyl)₂, C₁₋₆alkyl, C₁₋₆haloalkyl, and C₁₋₆alkoxy. In some embodiments is a compound of Formula (VI), wherein R¹ is unsubstituted C₂₋₉heteroaryl.

In some embodiments is a compound of Formula (VI), wherein R¹ is —CH₃. In some embodiments is a compound of Formula (VI), wherein R¹ is

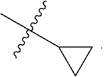 [SMILES: *C1CC1]

In some embodiments is a compound of Formula (VI), wherein R¹ is

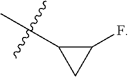 [SMILES: *C1CC1F]

In some embodiments is a compound of Formula (VI), wherein R¹ is

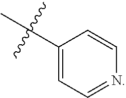 [SMILES: *c1ccncc1]

In some embodiments is a compound of Formula (VI), wherein R¹ is

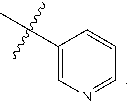 [SMILES: *c1cccnc1]

In some embodiments is a compound of Formula (VI), wherein R¹ is

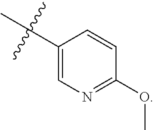 [SMILES: *c1ccc(OC)nc1]

In some embodiments is a compound of Formula (VI), wherein R¹ is

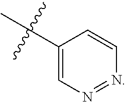 [SMILES: *c1ccnnc1]

In some embodiments is a compound of Formula (VI), wherein R¹ is

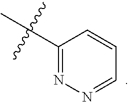 [SMILES: *c1cccnn1]

In some embodiments is a compound of Formula (VI), wherein R¹ is

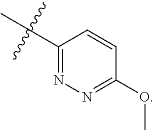 [SMILES: *c1ccc(OC)nn1]

In some embodiments is a compound of Formula (VI), wherein R¹ is

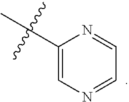 [SMILES: *c1cnccn1]

In some embodiments is a compound of Formula (VI), wherein R¹ is

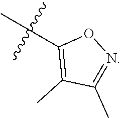 [SMILES: *c1onc(C)c1C]

In some embodiments the compound of Formula (VI) is selected from:

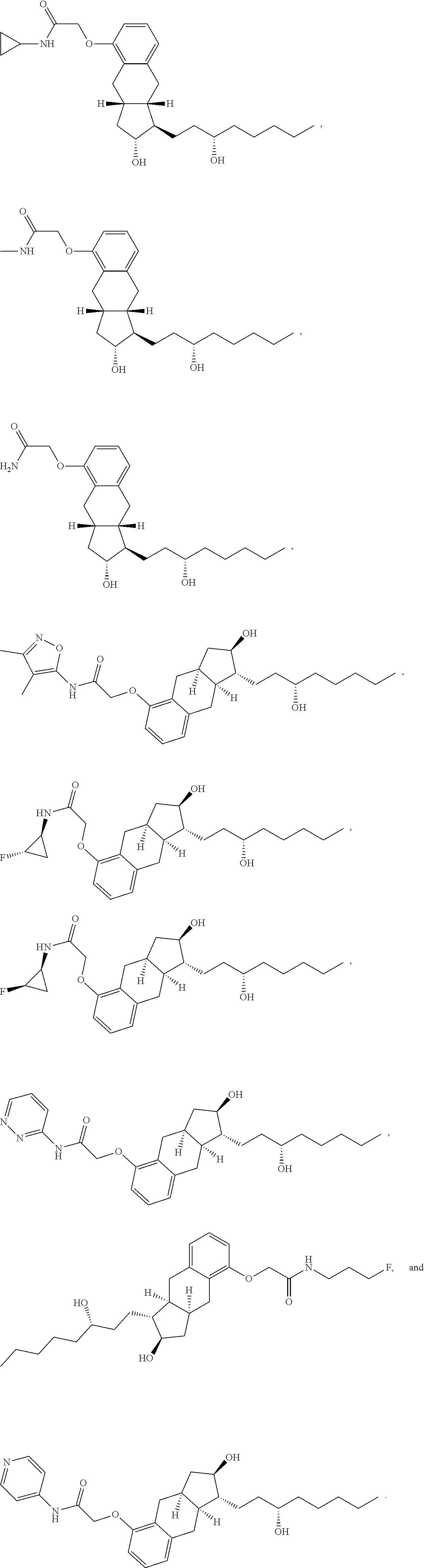

In some embodiments is a pharmaceutical composition comprising a compound of Formula (II), (III), (IV), (V), or (VI), or a pharmaceutically acceptable salt or solvate thereof, and a pharmaceutically acceptable excipient. In some embodiments is a pharmaceutical composition comprising a compound of Formula (II), (III), (IV), (V), or (VI), or a pharmaceutically acceptable salt or solvate thereof, and a pharmaceutically acceptable excipient, further comprising a peripherally restricted FAAH inhibitor. In some embodiments is a pharmaceutical composition comprising a compound of Formula (II), (III), (IV), (V), or (VI), or a pharmaceutically acceptable salt or solvate thereof, and a pharmaceutically acceptable excipient, further comprising a peripherally restricted FAAH inhibitor, wherein the peripherally restricted FAAH inhibitor is ASP-3652.

### Peripherally Restricted FAAH Inhibitors

In some embodiments, the pharmaceutical compositions described herein comprise a peripherally restricted FAAH inhibitor. In some embodiments, the peripherally restricted FAAH inhibitor is disclosed in US 2008/0306046 which is herein incorporated by reference in its entirety.

In some embodiments, the peripherally restricted FAAH inhibitor is a compound of Formula (X), or a pharmaceutically acceptable salt thereof:

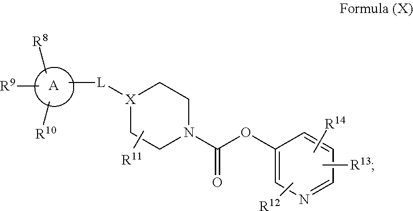

wherein:
ring A is a benzene ring, cyclopentane ring, cyclohexane ring, cycloheptane ring, or a 5- to 7 membered nitrogen-containing hetero ring;
L is a single bond, lower alkylene, lower alkenylene, —N(R¹⁵)—C(═O)—, —C(═O)—N(R¹⁵)—, -(lower alkenylene)-C(═O), —O—, or C(═O);
R¹⁵ is H or lower alkyl;
X is CH or N;
R⁸, R⁹, and R¹⁰ are each independently selected from:
(i) a group selected from the group consisting of H, halo, —CN, CF₃, lower alkyl, and —O-lower alkyl;
(ii) aryl optionally substituted with 1 to 5 groups independently selected from the group consisting of H, halo, —CN, CF₃, lower alkyl, and —O-lower alkyl;
(iii) nitrogen-containing heteroaryl optionally substituted with 1 to 5 groups independently selected from the group consisting of H, halo, —CN, —CF₃, lower alkyl, and —O-lower alkyl;
(iv) R¹⁶-(lower alkenylene)-O—;
(v) R¹⁶-(lower alkenylene)-N(R¹⁵)—; or
(vi) R¹⁷R¹⁸N—C(═O)—;
R¹⁶ is
(i) aryl optionally substituted with 1 to 5 groups independently selected from the group consisting of H, halo, —CN, —CF₃, lower alkyl, and —O-lower alkyl;
(ii) nitrogen-containing heteroaryl optionally substituted with 1 to 5 groups independently selected from the group consisting of H, halo, —CN, —CF₃, lower alkyl, and —O-lower alkyl; or (iii) 3- to 8-membered cycloalkyl;
R¹⁷ and R¹⁸ are each independently selected from H, lower alkyl, and 3- to 8-membered cycloalkyl; or R¹⁷ and R¹⁸ may form, together with the nitrogen atom bonded thereto, a 3- to 8-membered nitrogen-containing hetero ring;
R¹¹ is selected from H, lower alkyl, and oxo (═O); and
one of R¹², R¹³, and R¹⁴ is —C(═O)—O-(lower alkyl) or —CO₂H, and the others are H.

In some embodiments, the peripherally restricted FAAH inhibitor is 5-(((4-(4-((3-fluorobenzyl)oxy)phenoxy)piperidin-1-yl)carbonyl)oxy)nicotinic acid. In some embodiments, the peripherally restricted FAAH inhibitor is 5-(((4-(2-phenylethyl)piperidin-1-yl)carbonyl)oxy)nicotinic acid. In some embodiments, the peripherally restricted FAAH inhibitor is 5-(((4-(4-(2-cyclohexylethoxy)phenoxy)piperidin-1-yl)carbonyl)oxy)nicotinic acid. In some embodiments, the peripherally restricted FAAH inhibitor is 5-(((4-((E)-2-phenylvinyl)piperidin-1-yl)carbonyl)oxy)nicotinic acid. In some embodiments, the peripherally restricted FAAH inhibitor is 5-(((4-(3-(1-(6-methylpyridin-2-yl)piperidin-4-yl)propyl)piperidin-1-yl)carbonyl)oxy)nicotinic acid. In some embodiments, the peripherally restricted FAAH inhibitor is 5-(methoxycarbonyl)pyridin-3-yl 4-(2-phenylethyl)piperazine-1-carboxylate. In some embodiments, the peripherally restricted FAAH inhibitor is ASP-3652. In some embodiments, the peripherally restricted FAAH inhibitor is ASP-3652 which is 5-(((4-(2-phenylethyl)piperidin-1-yl)carbonyl)oxy)nicotinic acid.

### Methods

In some embodiments is a method of treating a CNS disease or disorder in a patient in need thereof comprising administering to the patient a pharmaceutical composition described herein comprising a fatty acid amide hydrolase (FAAH) cleavable prodrug of Formula (I), or a pharmaceutically acceptable salt or solvate thereof, and a pharmaceutically acceptable excipient; further comprising a peripherally restricted FAAH inhibitor. In some embodiments is a method of treating a CNS disease or disorder in a patient in need thereof comprising administering to the patient a pharmaceutical composition described herein comprising a fatty acid amide hydrolase (FAAH) cleavable prodrug of Formula (I), or a pharmaceutically acceptable salt or solvate thereof, and a pharmaceutically acceptable excipient; further comprising the peripherally restricted FAAH inhibitor ASP-3652. In some embodiments is a method of treating a CNS disease or disorder in a patient in need thereof comprising administering to the patient a pharmaceutical composition described herein comprising a fatty acid amide hydrolase (FAAH) cleavable prodrug of Formula (I), or a pharmaceutically acceptable salt or solvate thereof, and a pharmaceutically acceptable excipient; further comprising a peripherally restricted FAAH inhibitor, wherein the CNS disease or disorder is selected from multiple sclerosis, amyotrophic lateral sclerosis, Huntington's disease, Parkinson's disease, Alzheimer's disease, epilepsy, ischemic stroke, traumatic brain injury, autoimmune encephalomyelitis, schizophrenia, depression, mood disorders, attention deficit hyperactivity disorder, post-traumatic stress disorder, familial amyloidotic polyneuropathy, familial leptomeningeal amyloidosis, dementia, transitory focal neurological episodes, cognitive dysfunction, CNS amyloidosis, Degos disease, reversible cerebral vasoconstriction syndrome, Sneddon's syndrome, amyloid-beta-related angiopathy, Susac syndrome, and neurosarcoidosis. In some embodiments is a method of treating a CNS disease or disorder in a patient in need thereof comprising administering to the patient a pharmaceutical composition described herein comprising a fatty acid amide hydrolase (FAAH) cleavable prodrug of Formula (I), or a pharmaceutically acceptable salt or solvate thereof, and a pharmaceutically acceptable excipient; further comprising the peripherally restricted FAAH inhibitor ASP-3652, wherein the CNS disease or disorder is selected from multiple sclerosis, amyotrophic lateral sclerosis, Huntington's disease, Parkinson's disease, Alzheimer's disease, epilepsy, ischemic stroke, traumatic brain injury, autoimmune encephalomyelitis, schizophrenia, depression, mood disorders, attention deficit hyperactivity disorder, post-traumatic stress disorder, familial amyloidotic polyneuropathy, familial leptomeningeal amyloidosis, dementia, transitory focal neurological episodes, cognitive dysfunction, CNS amyloidosis, Degos disease, reversible cerebral vasoconstriction syndrome, Sneddon's syndrome, amyloid-beta-related angiopathy, Susac syndrome, and neurosarcoidosis.

In some embodiments is a method of treating a CNS disease or disorder in a patient in need thereof comprising administering to the patient a compound of Formula (II), or a pharmaceutically acceptable salt or solvate thereof. In some embodiments is a method of treating a CNS disease or disorder in a patient in need thereof comprising administering to the patient a pharmaceutical composition comprising a compound of Formula (II), or a pharmaceutically acceptable salt or solvate thereof, and a pharmaceutically acceptable excipient. In some embodiments is a method of treating a CNS disease or disorder in a patient in need thereof comprising administering to the patient a pharmaceutical composition comprising a compound of Formula (II), or a pharmaceutically acceptable salt or solvate thereof, and a pharmaceutically acceptable excipient, further comprising a peripherally restricted FAAH inhibitor. In some embodiments is a method of treating a CNS disease or disorder in a patient in need thereof comprising administering to the patient a pharmaceutical composition comprising a compound of Formula (II), or a pharmaceutically acceptable salt or solvate thereof, and a pharmaceutically acceptable excipient, further comprising the peripherally restricted FAAH inhibitor ASP-3652. In some embodiments, the CNS disease or disorder is selected from multiple sclerosis, amyotrophic lateral sclerosis, Huntington's disease, Parkinson's disease, and Alzheimer's disease.

In some embodiments is a method of treating a CNS disease or disorder in a patient in need thereof comprising administering to the patient a compound of Formula (III), or a pharmaceutically acceptable salt or solvate thereof. In some embodiments is a method of treating a CNS disease or disorder in a patient in need thereof comprising administering to the patient a pharmaceutical composition comprising a compound of Formula (III), or a pharmaceutically acceptable salt or solvate thereof, and a pharmaceutically acceptable excipient. In some embodiments is a method of treating a CNS disease or disorder in a patient in need thereof comprising administering to the patient a pharmaceutical composition comprising a compound of Formula (III), or a pharmaceutically acceptable salt or solvate thereof, and a pharmaceutically acceptable excipient, further comprising a peripherally restricted FAAH inhibitor. In some embodiments is a method of treating a CNS disease or disorder in a patient in need thereof comprising administering to the patient a pharmaceutical composition comprising a compound of Formula (III), or a pharmaceutically acceptable salt or solvate thereof, and a pharmaceutically acceptable excipient, further comprising the peripherally restricted FAAH inhibitor ASP-3652. In some embodiments, the CNS disease or disorder is selected from epilepsy, ischemic stroke, traumatic brain injury, and autoimmune encephalomyelitis.

In some embodiments is a method of treating a CNS disease or disorder in a patient in need thereof comprising administering to the patient a compound of Formula (IV), or a pharmaceutically acceptable salt or solvate thereof. In some embodiments is a method of treating a CNS disease or disorder in a patient in need thereof comprising administering to the patient a pharmaceutical composition comprising a compound of Formula (IV), or a pharmaceutically acceptable salt or solvate thereof, and a pharmaceutically acceptable excipient. In some embodiments is a method of treating a CNS disease or disorder in a patient in need thereof comprising administering to the patient a pharmaceutical composition comprising a compound of Formula (IV), or a pharmaceutically acceptable salt or solvate thereof, and a pharmaceutically acceptable excipient, further comprising a peripherally restricted FAAH inhibitor. In some embodiments is a method of treating a CNS disease or disorder in a patient in need thereof comprising administering to the patient a pharmaceutical composition comprising a compound of Formula (IV), or a pharmaceutically acceptable salt or solvate thereof, and a pharmaceutically acceptable excipient, further comprising the peripherally restricted FAAH inhibitor ASP-3652. In some embodiments, the CNS disease or disorder is selected from ischemic stroke, schizophrenia, depression, mood disorders, attention deficit hyperactivity disorder, post-traumatic stress disorder, and Alzheimer-type dementia.

In some embodiments is a method of treating a CNS disease or disorder in a patient in need thereof comprising administering to the patient a compound of Formula (V), or a pharmaceutically acceptable salt or solvate thereof. In some embodiments is a method of treating a CNS disease or disorder in a patient in need thereof comprising administering to the patient a pharmaceutical composition comprising a compound of Formula (V), or a pharmaceutically acceptable salt or solvate thereof, and a pharmaceutically acceptable excipient. In some embodiments is a method of treating a CNS disease or disorder in a patient in need thereof comprising administering to the patient a pharmaceutical composition comprising a compound of Formula (V), or a pharmaceutically acceptable salt or solvate thereof, and a pharmaceutically acceptable excipient, further comprising a peripherally restricted FAAH inhibitor. In some embodiments is a method of treating a CNS disease or disorder in a patient in need thereof comprising administering to the patient a pharmaceutical composition comprising a compound of Formula (V), or a pharmaceutically acceptable salt or solvate thereof, and a pharmaceutically acceptable excipient, further comprising the peripherally restricted FAAH inhibitor ASP-3652. In some embodiments, the CNS disease or disorder is selected from familial amyloidotic polyneuropathy, familial leptomeningeal amyloidosis, Alzheimer's disease, stroke, dementia, transitory focal neurological episodes, cognitive dysfunction, and CNS amyloidosis.

In some embodiments is a method of treating a CNS disease or disorder in a patient in need thereof comprising administering to the patient a compound of Formula (VI), or a pharmaceutically acceptable salt or solvate thereof. In some embodiments is a method of treating a CNS disease or disorder in a patient in need thereof comprising administering to the patient a pharmaceutical composition comprising a compound of Formula (VI), or a pharmaceutically acceptable salt or solvate thereof, and a pharmaceutically acceptable excipient. In some embodiments is a method of treating a CNS disease or disorder in a patient in need thereof comprising administering to the patient a pharmaceutical composition comprising a compound of Formula (VI), or a pharmaceutically acceptable salt or solvate thereof, and a pharmaceutically acceptable excipient, further comprising a peripherally restricted FAAH inhibitor. In some embodiments is a method of treating a CNS disease or disorder in a patient in need thereof comprising administering to the patient a pharmaceutical composition comprising a compound of Formula (VI), or a pharmaceutically acceptable salt or solvate thereof, and a pharmaceutically acceptable excipient, further comprising the peripherally restricted FAAH inhibitor ASP-3652. In some embodiments, the CNS disease or disorder is selected from Degos disease, reversible cerebral vasoconstriction syndrome, Sneddon's syndrome, amyloid-beta-related angiopathy, Susac syndrome, and neurosarcoidosis.

### Excipients

Suitable optional excipients for use in the pharmaceutical compositions described herein include any commonly used excipients in pharmaceutics and are selected on the basis of compatibility with the active pharmaceutical agent and the release profile properties of the desired dosage form. Excipients include, but are not limited to, binders, fillers, flow aids, disintegrants, lubricants, glidants, polymeric carriers, plasticizers, stabilizers, surfactants, and the like. A summary of excipients described herein, may be found, for example in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999), herein incorporated by reference in their entirety.

Binders impart cohesiveness to solid oral dosage form formulations: for powder filled capsule formulation, they aid in plug formation that can be filled into soft or hard shell capsules and for tablet formulation, they ensure the tablet remaining intact after compression and help assure blend uniformity prior to a compression or fill step. Materials suitable for use as binders in the solid dosage forms described herein include, but are not limited to, carboxymethylcellulose, methylcellulose (e.g., Methocel®), hydroxypropylmethylcellulose (e.g.

Hypromellose USP Pharmacoat-603, hydroxypropylmethylcellulose acetate stearate (Agoate HS-LF and HS), hydroxyethylcellulose, hydroxypropylcellulose (e.g., Klucel®), ethylcellulose (e.g., Ethocel®), and microcrystalline cellulose (e.g., Avicel®), microcrystalline dextrose, amylose, magnesium aluminum silicate, polysaccharide acids, bentonites, gelatin, polyvinyl pyrrolidone/vinyl acetate copolymer, crospovidone, povidone, starch, pregelatinized starch, tragacanth, dextrin, a sugar, such as sucrose (e.g., Dipac®), glucose, dextrose, molasses, mannitol, sorbitol, xylitol (e.g., Xylitab®), lactose, a natural or synthetic gum such as acacia, tragacanth, ghatti gum, mucilage of isapol husks, starch, polyvinyl pyrrolidone (e.g., Povidone® CL, Kollidon® CL, Polyplasdone® XL-10, and Povidone® K-12), larch arabogalactan, Veegum®, polyethylene glycol, waxes, sodium alginate, and the like.

Fillers or diluents increase bulk in the pharmaceutical formulation. Such compounds include e.g., lactose; starch; mannitol; sorbitol; dextrose; microcrystalline cellulose such as Avicel®; dibasic calcium phosphate; dicalcium phosphate dihydrate; tricalcium phosphate; calcium phosphate; anhydrous lactose; spray-dried lactose; pregelatinzed starch; compressible sugar, such as Di-Pac® (Amstar); hydroxypropylmethylcellulose; sucrose-based diluents; confectioner's sugar; monobasic calcium sulfate monohydrate; calcium sulfate dihydrate; calcium lactate trihydrate; dextrates; hydrolyzed cereal solids; amylose; powdered cellulose; calcium carbonate; glycine; kaolin; sodium chloride; inositol; bentonite; and the like.

Glidants improve the flow characteristics of a powder mixtures. Such compounds include, e.g., colloidal silicon dioxide such as Cab-o-Sil®; tribasic calcium phosphate, talc, corn starch, DL-leucine, sodium lauryl sulfate, magnesium stearate, calcium stearate, sodium stearate, kaolin, and micronized amorphous silicon dioxide (Syloid®) and the like.

Lubricants are compounds which prevent, reduce, or inhibit adhesion or friction of materials. Exemplary lubricants include, e.g., stearic acid; calcium hydroxide, talc; a hydrocarbon such as mineral oil, or hydrogenated vegetable oil such as hydrogenated soybean oil (Sterotex®), Lubritab®, Cutina®; higher fatty acids and their alkali-metal and alkaline earth metal salts, such as aluminum, calcium, magnesium, zinc, stearic acid, sodium stearates, magnesium stearate, glycerol, talc, waxes, Stearowet®, boric acid, sodium acetate, leucine, a polyethylene glycol or a methoxypolyethylene glycol such as Carbowax™, sodium oleate, glyceryl behenate (Compitrol 888®), glyceryl palmitostearate (Precirol®), colloidal silica such as Syloid™, Carb-O-Sil®, a starch such as corn starch, silicone oil, a surfactant, and the like. Hydrophilic lubricants include, e.g., sodium stearyl fumarate (currently marketed under the trade name PRUV®), polyethylene glycol (PEG), magnesium lauryl sulfate, sodium lauryl sulfate (SLS), sodium benzoate, sodium chloride, and the like.

Disintegrants facilitate breakup or disintegration of the pharmaceutical formulation after administration. Examples of disintegrants include a starch, e.g., a natural starch such as corn starch or potato starch, a pregelatinized starch such as National 1551 or Amijel®, or sodium starch glycolate such as Promogel® or Explotab®; a cellulose such as a wood product, microcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose; a cross-linked starch such as sodium starch glycolate; a cross-linked polymer such as crospovidone; a cross-linked polyvinyl pyrrolidone; alginate such as alginic acid or a salt of alginic acid such as sodium alginate; a clay such as Veegum® HV (magnesium aluminum silicate); a gum such as agar, guar, locust bean, Karaya, pectin, or tragacanth; sodium starch glycolate; bentonite; a natural sponge; a resin such as a cation-exchange resin; citrus pulp; sodium lauryl sulfate; sodium lauryl sulfate in combination starch; and the like.

Polymeric carriers include compounds such as polyvinyl pyrrolidone, e.g., polyvinyl pyrrolidone K12, polyvinyl pyrrolidone K17, polyvinyl pyrrolidone K25, or polyvinyl pyrrolidone K30, polyvinyl pyrrolidone vinyl acetate (PVPVA 64), hydroxypropylmethylcellulose (HPMC), hydroxypropylmethylcellulose acetylsuccinate (HPMC AS), and methylmethacrylate polymers (Eudragit polymers) and the like.

Stabilizers include compounds such as any anti-oxidation agents, e.g., butylated hydroxytoluene (BHT), sodium ascorbate, and tocopherol; buffers, acids, and the like.

Surfactants include compounds such as sodium lauryl sulfate, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), d-α-tocopheryl polyethylene glycol succinate (Vitamin E TPGS); and the like.

The aforementioned excipients are given as examples only and are not meant to include all possible choices. Other suitable excipient classes include coloring agents, granulating agents, preservatives, anti-foaming agents, plasticizers, and the like. Additionally, many excipients can have more than one role or function, or can be classified in more than one group; the classifications are descriptive only, and are not intended to limit any use of a particular excipient.

Disclosed pharmaceutical formulations are administered to patients (animals and humans) in need of such treatment in dosages that will provide optimal pharmaceutical efficacy. It will be appreciated that the dose required for use in any particular application will vary from patient to patient, not only with the particular pharmaceutical formulation selected, but also with the nature of the condition being treated, the age and condition of the patient, concurrent medication or special diets then being followed by the patient, and other factors, with the appropriate dosage ultimately being at the discretion of the attendant physician.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

The following examples are offered for purposes of illustration and are not intended to limit the scope of the claims provided herein. All literature citations in these examples and throughout this specification are incorporated herein by references for all legal purposes to be served thereby. The starting materials and reagents used for the synthesis of the compounds described herein may be synthesized or can be obtained from commercial sources, such as, but not limited to, Sigma-Aldrich, Acros Organics, Fluka, and Fischer Scientific.

As used above, and throughout the description of the invention, the following abbreviations, unless otherwise indicated, shall be understood to have the following meanings:
ACN or MeCN acetonitrile
AcOH acetic acid
Ac acetyl
BINAP 2,2′-bis(diphenylphosphino)-1,1′-binaphthalene
Bn benzyl
BOC or Boc tert-butyl carbamate
t-Bu tert-butyl
Cy cyclohexyl
DBA or dba dibenzylideneacetone
CDI 1,1-carbonyldiimidazole
DCE dichloroethane (ClCH₂CH₂Cl)
DCM dichloromethane (CH₂Cl₂)
DIPEA or DIEA diisopropylethylamine
DMAP 4-(N,N-dimethylamino)pyridine
DME 1,2-dimethoxyethane
DMF N,N-dimethylformamide
DMA N,N-dimethylacetamide
DMSO dimethylsulfoxide
Dppf or dppf 1,1′-bis(diphenylphosphino)ferrocene
EDC or EDCI N-(3-dimethylaminopropyl)-N′-ethylcarbodiimide
hydrochloride
eq equivalent(s)
Et ethyl
Et₂O diethyl ether
EtOH ethanol
EtOAc ethyl acetate
HATU 1-[bis(dimethylamino)methylene]-1H-1,2,3-triazolo[4,5-b]pyridinium 3-oxid hexafluorophosphate
HMPA hexamethylphosphoramide
HOBt 1-hydroxybenzotriazole
HPLC high performance liquid chromatography
KHMDS potassium bis(trimethylsilyl)amide
NaHMDS sodium bis(trimethylsilyl)amide
LiHMDS lithium bis(trimethylsilyl)amide
LAH lithium aluminum anhydride
LCMS liquid chromatography mass spectrometry
Me methyl
MeOH methanol
MS mass spectroscopy
Ms mesyl
MTBE methyl tert-butyl ether
NBS N-bromosuccinimide
NMM N-methyl-morpholine
NMP N-methyl-pyrrolidin-2-one
NMR nuclear magnetic resonance
Ph phenyl
PPTS pyridium p-toluenesulfonate
iPr/i-Pr iso-propyl
rt room temperature
TFA trifluoroacetic acid
TEA triethylamine
THE tetrahydrofuran
TLC thin layer chromatography

### Example 1: Synthesis of 3-(4-((5-chloro-2,2-dimethyl-2,3-dihydrobenzofuran-7-yl)methoxy)-2,3-dimethylphenyl)-N-(pyridazin-3-yl)propenamide (2)

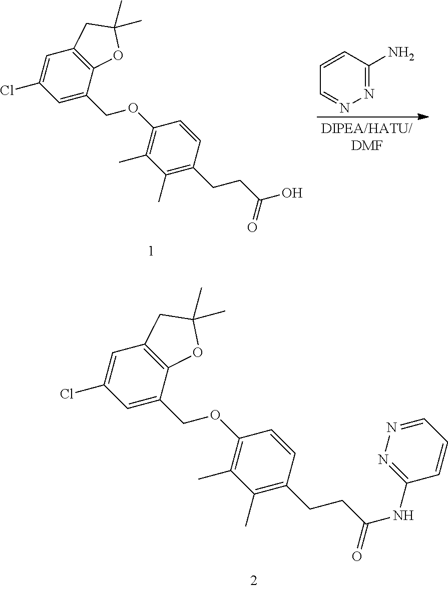 [SMILES: Cc1c(CCC(=O)O)ccc(OCc2cc(Cl)cc3c2OC(C)(C)C3)c1C ; Nc1cccnn1 ; Cc1c(CCC(=O)Nc2cccnn2)ccc(OCc2cc(Cl)cc3c2OC(C)(C)C3)c1C]

To a solution of compound 1 (50.0 mg, 128.5 μmol) in DMF (2 mL) at rt was added DIPEA (33.2 mg, 257.1 μmol), HATU (73.3 mg, 192.8 μmol), and pyridazin-3-amine (36.6 mg, 385.7 μmol). The mixture was stirred at rt overnight. Water (20 mL) was added and the mixture was extracted with EtOAc (10 mL*2). The combined organic layer was washed with water (10 mL*2), brine (20 mL), dried over Na₂SO₄, concentrated to dryness and purified by prep-HPLC to afford compound 2 (15 mg, 25.0% yield) as a yellow solid. LCMS: M+H=466.2.

### Example 2: Synthesis of 3-(4-((5-chloro-2,2-dimethyl-2,3-dihydrobenzofuran-7-yl)methoxy)-2,3-dimethylphenyl)-N-cyclopropylpropanamide (3)

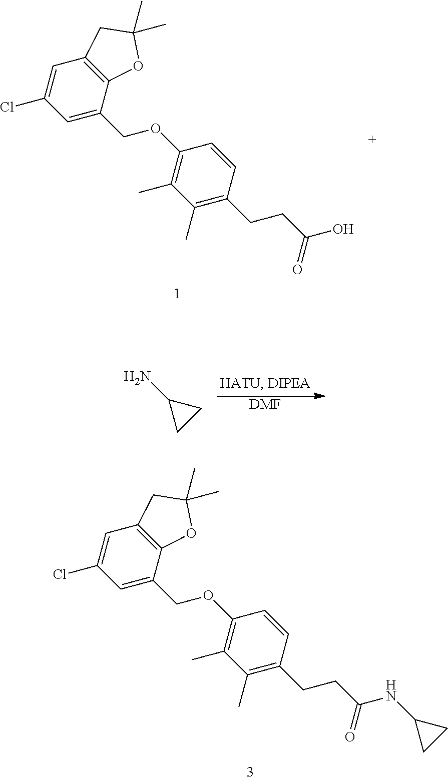

To a solution of compound 1 (50.0 mg, 0.13 mmol) in DMF (2 mL) at rt was added DIPEA (34.0 mg, 0.26 mmol), HATU (74.0 mg, 0.2 mmol), and cyclopropylamine (7.0 mg, 0.13 mol). The mixture was stirred at rt overnight. H₂O (20 mL) was added, the mixture was extracted with EtOAc (10 mL*2). The combined organic layer was washed with water (10 mL*2), brine (20 mL), dried over Na₂SO₄ and concentrated to dryness. The residue was purified by prep-HPLC to afford compound 3 (20 mg, 36.0% yield) as a white solid. LCMS: M+H=428.2.

### Example 3: Synthesis of 3-(4-((5-chloro-2,2-dimethyl-2,3-dihydrobenzofuran-7-yl)methoxy)-2,3-dimethylphenyl)-N-((1R,2R)-2-fluorocyclopropyl)propenamide (4) and 3-(4-((5-chloro-2,2-dimethyl-2,3-dihydrobenzofuran-7-yl)methoxy)-2,3-dimethylphenyl)-N-((1R,2S)-2-fluorocyclopropyl)propenamide (5)

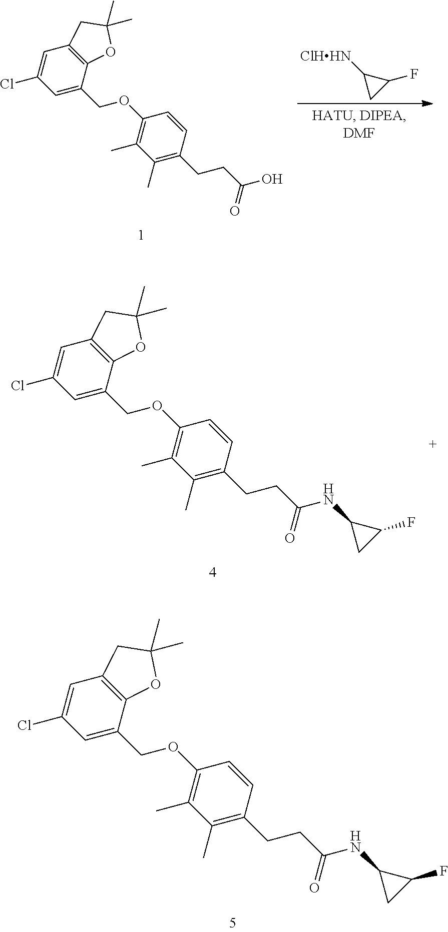

To a solution of compound 1 (150 mg, 0.39 mmol) in DMF (3 mL) at rt was added DIPEA (151 mg, 1.17 mmol), HATU (222 mg, 0.59 mmol), and 2-fluorocyclopropan-1-amine hydrochloride (47 mg, 0.42 mol). The reaction mixture was stirred at rt overnight. H₂O (20 mL) was added. The mixture was extracted with EtOAc (20 mL*2) and the combined organic layer was washed with water (20 mL*2), brine (20 mL), dried over Na₂SO₄ and concentrated to dryness. The residue was purified by prep-HPLC to afford compound 4 (30 mg, 17.3% yield) and compound 5 (30 mg, 17.3% yield) as a white solid. LCMS (compound 4): M+H=446.2; LCMS (compound 5): M+1=446.2.

### Example 4: Synthesis of 3-(4-((5-chloro-2,2-dimethyl-2,3-dihydrobenzofuran-7-yl)methoxy)-2,3-dimethylphenyl)-N-(3-fluoropropyl)propenamide (6)

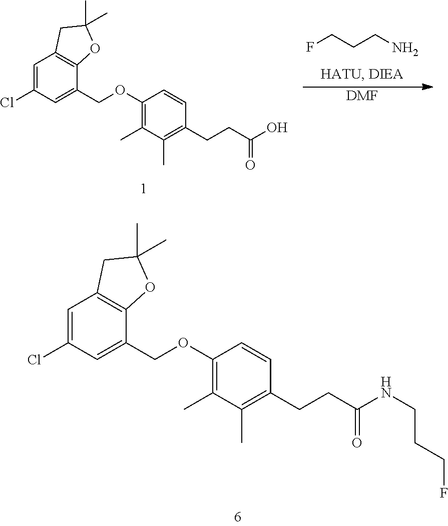

To a solution of compound 1 (200 mg, 514.29 μmol) and HATU (293.3 mg, 771.44 mol) in DMF (3 mL) was added DIEA (199.4 mg, 1.54 mmol) and 3-fluoropropan-1-amine (47.5 mg, 617.15 μmol). The mixture was stirred at room temperature 2 h. The mixture was filtered and purified by prep-HPLC to afford compound 6 (10 mg, 4.1% yield) as a white solid. LCMS: M+H=448.35.

### Example 5: Synthesis of 3-(4-((5-chloro-2,2-dimethyl-2,3-dihydrobenzofuran-7-yl)methoxy)-2,3-dimethylphenyl)-N-(pyridin-4-yl)propenamide (7)

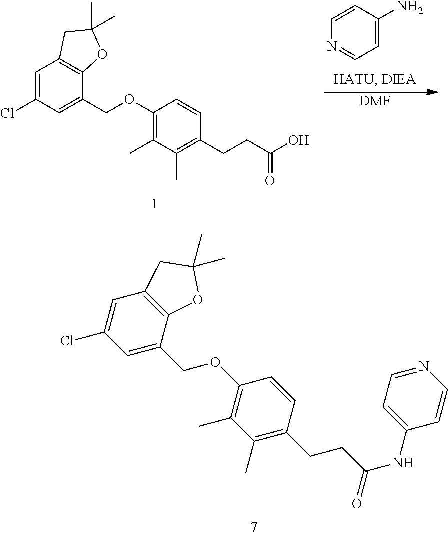

To a solution of compound 1 (50 mg, 128.57 μmol), pyridin-4-amine (12.1 mg, 128.57 mol) and HATU (73.3 mg, 192.86 μmol) in DMF (3 mL) was added DIEA (33.2 mg, 257.15 mol). The mixture was stirred at room temperature 2 h. The mixture was filtered and purified by prep-HPLC and RP-column to afford compound 7 (8 mg, 13.4% yield) as a white solid. LCMS: M+H=465.2.

### Example 6: Synthesis of 3-(4-((5-chloro-2,2-dimethyl-2,3-dihydrobenzofuran-7-yl)methoxy)-2,3-dimethylphenyl)-N-(3,4-dimethylisoxazol-5-yl)propenamide (8)

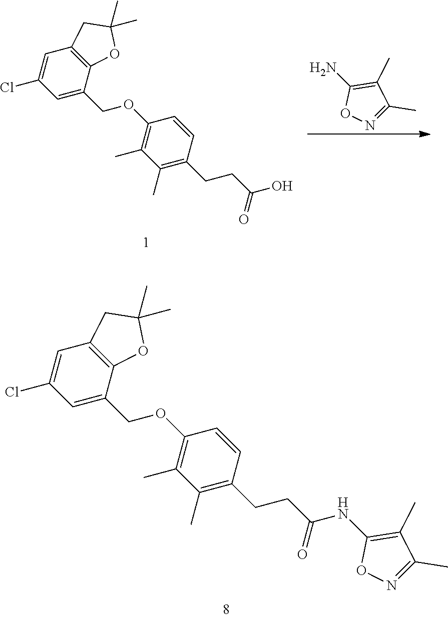

To a solution of compound 1 (50 mg, 128.57 umol) in DCM (5.0 mL) was added cat DMF and oxalyl chloride (129 mg, 1.0 mmol). The mixture was stirred at rt 2 h. The mixture was concentrated to dryness to afford acid chloride as colorless oil.

A solution of acid chloride (50 mg, 128.57 umol) in DCM (2.0 mL) was added to 3,4-dimethylisoxazol-5-amine (28 mg, 257.14 umol) and DIPEA (66 mg, 514.28 umol). The mixture was stirred at rt 1 h. Water (10 mL) was added and the mixture was extracted with DCM (10 mL*3). The combined organic phase was washed by brine (30 mL), dried over Na₂SO₄, concentrated in vacuum and purified by prep-HPLC to afford compound 8 (2 mg, 3.1% yield) as a white solid. LCMS: M−1=481.10.

### Example 7: Synthesis of 3-(4-((5-chloro-2,2-dimethyl-2,3-dihydrobenzofuran-7-yl)methoxy)-2,3-dimethylphenyl)propenamide (9)

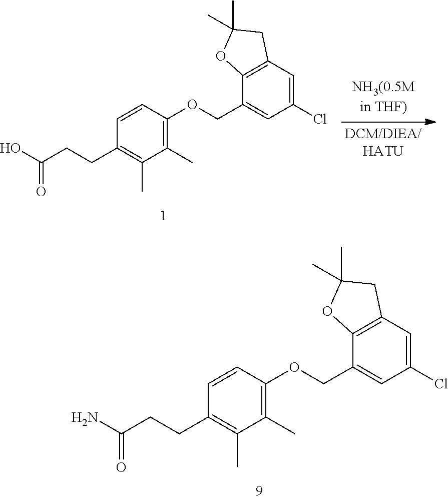 [SMILES: Cc1c(CCC(=O)O)ccc(OCc2cc(Cl)cc3c2OC(C)(C)C3)c1C ; Cc1c(CCC(N)=O)ccc(OCc2cc(Cl)cc3c2OC(C)(C)C3)c1C]

To a solution of compound 1 (100 mg, 257.14 μmol) in DCM (2 mL) at rt was added DIPEA (66.4 mg, 514.30 μmol), HATU (147.4 mg, 385.71 μmol) and NH₃ (36.0 mg, 1.028 mmol). The mixture was stirred at rt overnight. H₂O (20 mL) was added and extracted with DCM (10 mL*2). The combined organic layer was washed with water (10 mL*2), brine (20 mL), dried over Na₂SO₄, concentrated to dryness and purified by prep-HPLC to afford compound 9 (80 mg, 80.0% yield) as a white solid. LCMS: M+H=388.0.

### Example 8: Synthesis of 3-(4-((5-chloro-2,2-dimethyl-2,3-dihydrobenzofuran-7-yl)methoxy)-2,3-dimethylphenyl)-N-methylpropanamide (10)

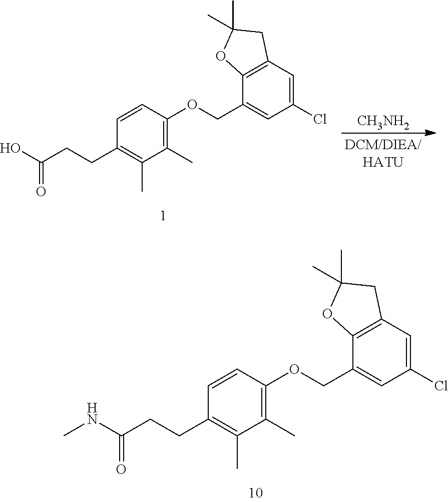 [SMILES: Cc1c(CCC(=O)O)ccc(OCc2cc(Cl)cc3c2OC(C)(C)C3)c1C ; "CH3NH2" ; CNC(=O)CCc1ccc(OCc2cc(Cl)cc3c2OC(C)(C)C3)c(C)c1C]

To a solution of compound 1 (100 mg, 257.1 μmol) in DCM (2 mL) at rt was added DIPEA (66.4 mg, 514.3 μmol), HATU (147.4 mg, 385.7 μmol), and CH₃NH₂ (31.9 mg, 1.0 mmol). The mixture was stirred at rt overnight. H₂O (20 mL) was added and the mixture was extracted with DCM (10 mL*2). The combined organic layer was washed with water (10 mL*2), brine (20 mL), dried over Na₂SO₄, concentrated to dryness and purified by prep-HPLC to afford product compound 10 (80 mg, 80.0% yield) as a white solid. LCMS: M+H=402.0.

### Example 9: Synthesis of 3-(4-((5-chloro-2,2-dimethyl-2,3-dihydrobenzofuran-7-yl)methoxy)-2,3-dimethylphenyl)-N-(2-hydroxyethyl)propenamide (11)

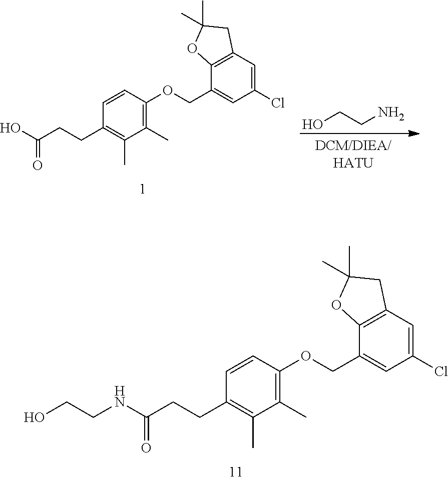

To a solution of compound 1 (100 mg, 257.1 μmol) in DCM (2 mL) at rt was added DIPEA (66.4 mg, 514.3 μmol), HATU (147.4 mg, 385.7 μmol) and 2-aminoethan-1-ol (62.8 mg, 1.03 mmol). The mixture was stirred at rt overnight. H₂O (20 mL) was added, the mixture was extracted with DCM (10 mL*2). The combined organic layer was washed with water (10 mL*2), brine (20 mL), dried over Na₂SO₄, concentrated to dryness and purified by prep-HPLC to afford compound 11 (70 mg, 63.0% yield) as a white solid. LCMS: M+H=431.2.

Compounds 12-57 in Table 1 were prepared as outlined in the preceding examples starting from the appropriate carboxylic acid.

TABLE 1

| | | LCMS |
|---|---|---|
| Compound | Structure | [M + H]⁺ |
| 12 | 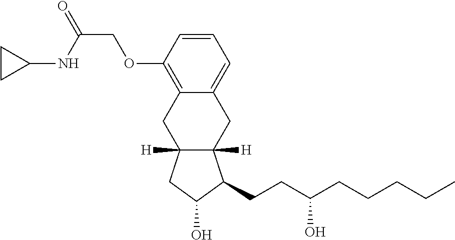 [SMILES: CCCCC[C@H](O)CC[C@@H]1[C@H]2Cc3cccc(OCC(=O)NC4CC4)c3C[C@H]2C[C@H]1O] | 452.0 (M + Na) |
| 13 | 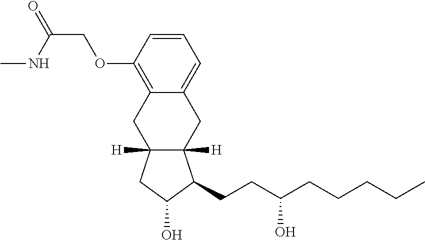 [SMILES: CCCCC[C@H](O)CC[C@@H]1[C@H]2Cc3cccc(OCC(=O)NC)c3C[C@H]2C[C@H]1O] | 426.0 (M + Na) |
| 14 | 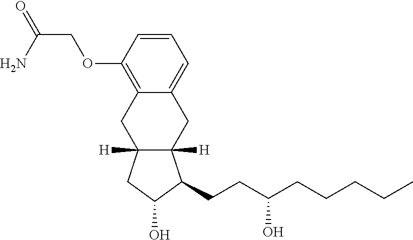 [SMILES: CCCCC[C@H](O)CC[C@@H]1[C@H]2Cc3cccc(OCC(N)=O)c3C[C@H]2C[C@H]1O] | 412.1 (M + Na) |
| 15 | 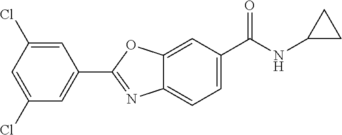 [SMILES: O=C(NC1CC1)c1ccc2nc(-c3cc(Cl)cc(Cl)c3)oc2c1] | 346.9 |
| 16 | 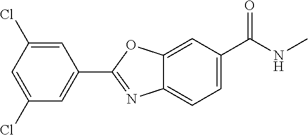 [SMILES: CNC(=O)c1ccc2nc(-c3cc(Cl)cc(Cl)c3)oc2c1] | 320.8 |
| 17 | 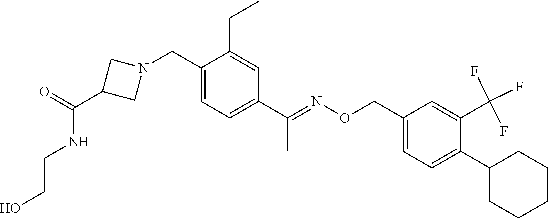 [SMILES: CCc1cc(/C(C)=N/OCc2ccc(C3CCCCC3)c(C(F)(F)F)c2)ccc1CN1CC(C(=O)NCCO)C1] | 560.0 |
| 18 | 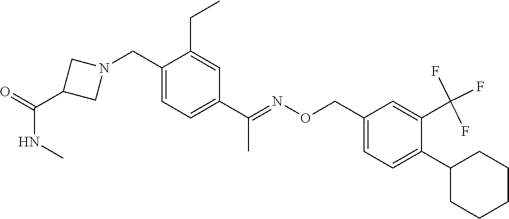 [SMILES: CCc1cc(/C(C)=N/OCc2ccc(C3CCCCC3)c(C(F)(F)F)c2)ccc1CN1CC(C(=O)NC)C1] | 530.0 |
| 19 | 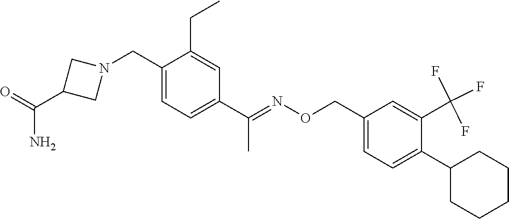 [SMILES: CCc1cc(/C(C)=N/OCc2ccc(C3CCCCC3)c(C(F)(F)F)c2)ccc1CN1CC(C(N)=O)C1] | 516.0 |
| 20 | 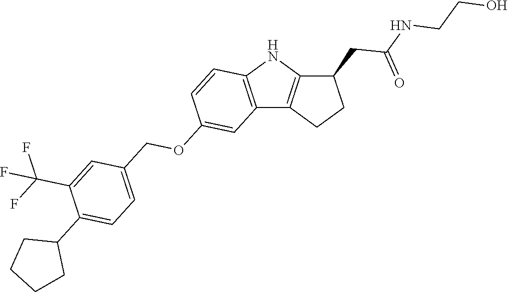 [SMILES: O=C(C[C@H]1CCc2c1[nH]c1ccc(OCc3ccc(C4CCCC4)c(C(F)(F)F)c3)cc21)NCCO] | 499.2 |
| 21 | 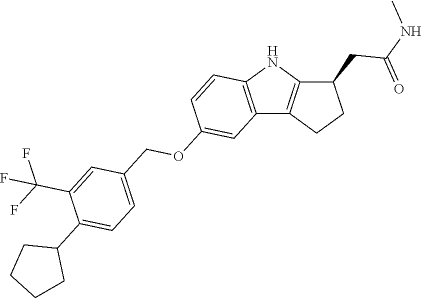 [SMILES: CNC(=O)C[C@H]1CCc2c1[nH]c1ccc(OCc3ccc(C4CCCC4)c(C(F)(F)F)c3)cc21] | 471.2 |
| 22 | 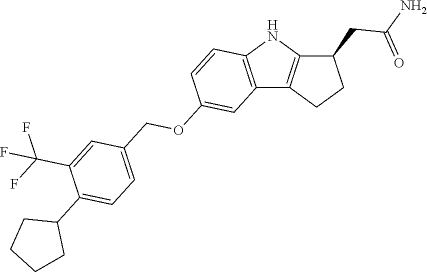 [SMILES: NC(=O)C[C@H]1CCc2c1[nH]c1ccc(OCc3ccc(C4CCCC4)c(C(F)(F)F)c3)cc21] | 455.1 |
| 23 | 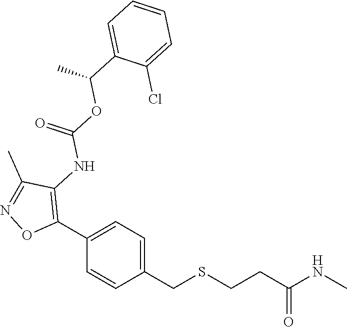 [SMILES: CNC(=O)CCSCc1ccc(-c2onc(C)c2NC(=O)O[C@H](C)c2ccccc2Cl)cc1] | 488.0 |
| 24 | 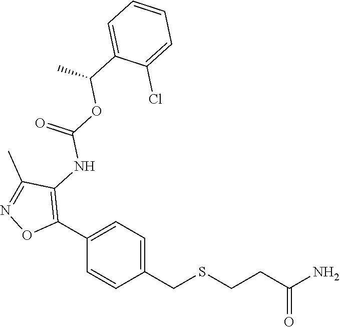 [SMILES: Cc1noc(-c2ccc(CSCCC(N)=O)cc2)c1NC(=O)O[C@H](C)c1ccccc1Cl] | 474.0 |
| 25 | 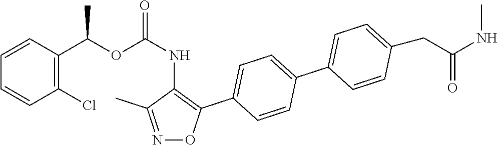 [SMILES: CNC(=O)Cc1ccc(-c2ccc(-c3onc(C)c3NC(=O)O[C@H](C)c3ccccc3Cl)cc2)cc1] | 502.3 |
| 26 | 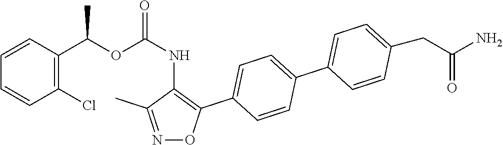 [SMILES: Cc1noc(-c2ccc(-c3ccc(CC(N)=O)cc3)cc2)c1NC(=O)O[C@H](C)c1ccccc1Cl] | 488.1 |
| 27 | 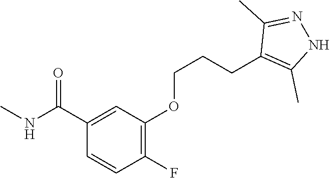 [SMILES: CNC(=O)c1ccc(F)c(OCCCc2c(C)n[nH]c2C)c1] | 306.1 |
| 28 | 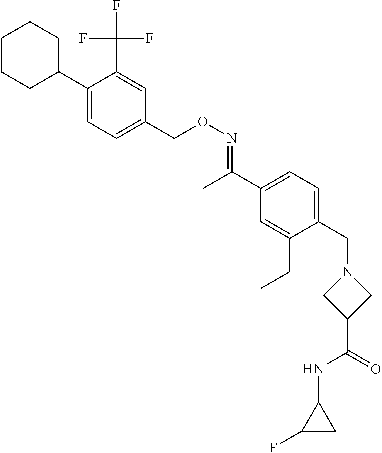 [SMILES: CCc1cc(/C(C)=N/OCc2ccc(C3CCCCC3)c(C(F)(F)F)c2)ccc1CN1CC(C(=O)NC2CC2F)C1] | 574.4 |
| 29 | 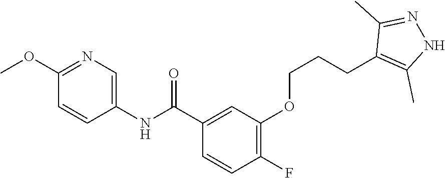 [SMILES: COc1ccc(NC(=O)c2ccc(F)c(OCCCc3c(C)n[nH]c3C)c2)cn1] | 399.2 |
| 30 | 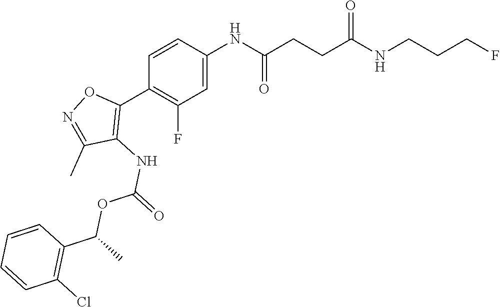 [SMILES: Cc1noc(-c2ccc(NC(=O)CCC(=O)NCCCF)cc2F)c1NC(=O)O[C@H](C)c1ccccc1Cl] | 549.2 |
| 31 | 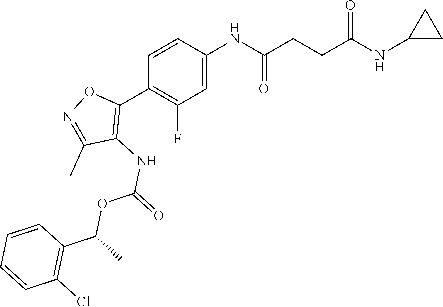 [SMILES: Cc1noc(-c2ccc(NC(=O)CCC(=O)NC3CC3)cc2F)c1NC(=O)O[C@H](C)c1ccccc1Cl] | 529.1 |
| 32 | 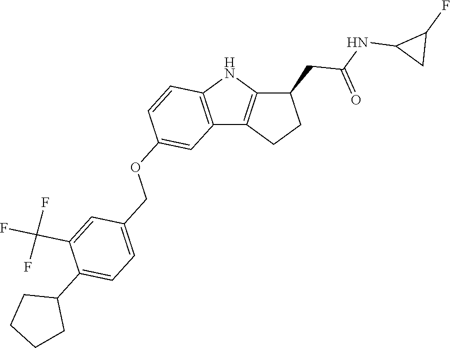 [SMILES: O=C(C[C@H]1CCc2c1[nH]c1ccc(OCc3ccc(C4CCCC4)c(C(F)(F)F)c3)cc21)NC1CC1F] | 515.3 |
| 33 | 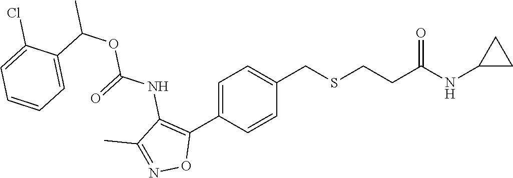 [SMILES: Cc1noc(-c2ccc(CSCCC(=O)NC3CC3)cc2)c1NC(=O)OC(C)c1ccccc1Cl] | 514.2 |
| 34 | 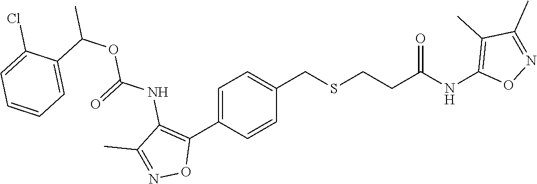 [SMILES: Cc1noc(NC(=O)CCSCc2ccc(-c3onc(C)c3NC(=O)OC(C)c3ccccc3Cl)cc2)c1C] | 569.2 |
| 35 | 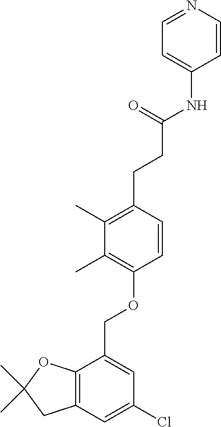 [SMILES: Cc1c(CCC(=O)Nc2ccncc2)ccc(OCc2cc(Cl)cc3c2OC(C)(C)C3)c1C] | 465.2 |
| 36 | 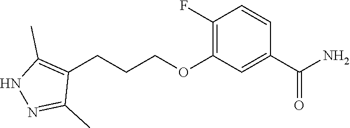 [SMILES: Cc1n[nH]c(C)c1CCCOc1cc(C(N)=O)ccc1F] | 292.2 |
| 37 | 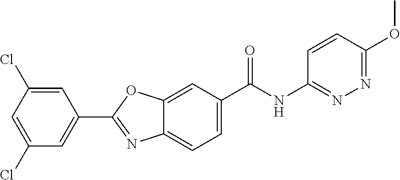 [SMILES: COc1ccc(NC(=O)c2ccc3nc(-c4cc(Cl)cc(Cl)c4)oc3c2)nn1] | 415.0 |
| 38 | 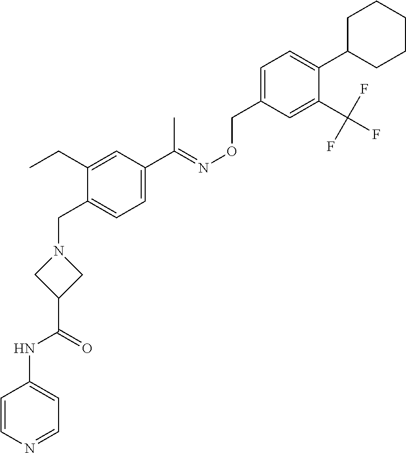 [SMILES: CCc1cc(/C(C)=N/OCc2ccc(C3CCCCC3)c(C(F)(F)F)c2)ccc1CN1CC(C(=O)Nc2ccncc2)C1] | 593.3 |
| 39 | 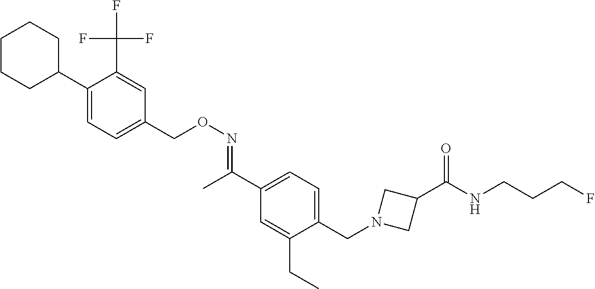 [SMILES: CCc1cc(/C(C)=N/OCc2ccc(C3CCCCC3)c(C(F)(F)F)c2)ccc1CN1CC(C(=O)NCCCF)C1] | 576.4 |
| 40 | 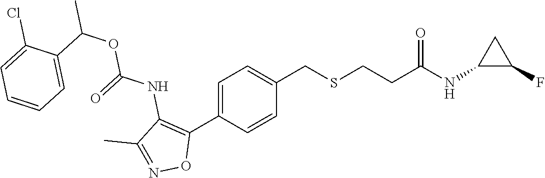 [SMILES: Cc1noc(-c2ccc(CSCCC(=O)N[C@@H]3C[C@H]3F)cc2)c1NC(=O)OC(C)c1ccccc1Cl] | 532.2 |
| 41 | 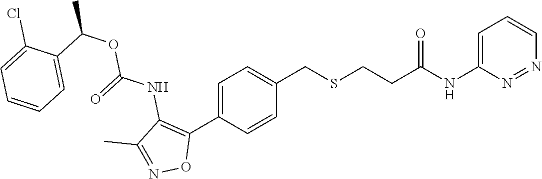 [SMILES: Cc1noc(-c2ccc(CSCCC(=O)Nc3cccnn3)cc2)c1NC(=O)O[C@H](C)c1ccccc1Cl] | 552.2 |
| 42 | 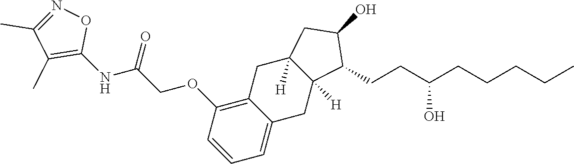 | 483.4 |
| 43 | 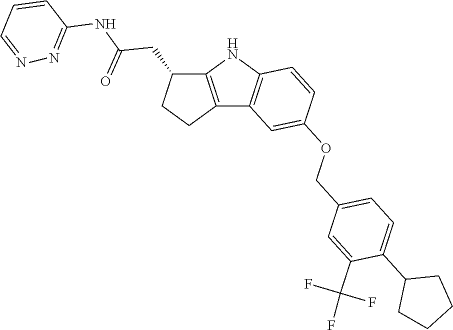 [SMILES: O=C(C[C@H]1CCc2c1[nH]c1ccc(OCc3ccc(C4CCCC4)c(C(F)(F)F)c3)cc21)Nc1cccnn1] | 533.2 (M − H) |
| 44 | 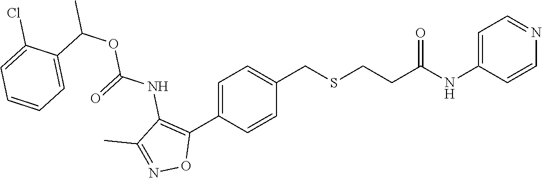 [SMILES: Cc1noc(-c2ccc(CSCCC(=O)Nc3ccncc3)cc2)c1NC(=O)OC(C)c1ccccc1Cl] | 551.1 |
| 45 | 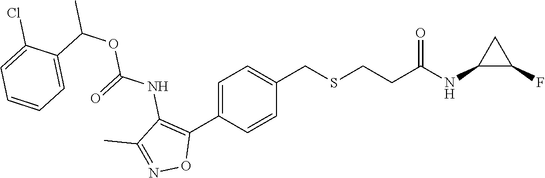 [SMILES: Cc1noc(-c2ccc(CSCCC(=O)N[C@H]3C[C@H]3F)cc2)c1NC(=O)OC(C)c1ccccc1Cl] | 532.2 |
| 46 | 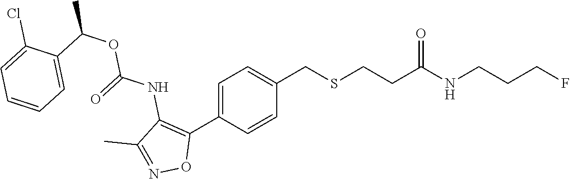 [SMILES: Cc1noc(-c2ccc(CSCCC(=O)NCCCF)cc2)c1NC(=O)O[C@H](C)c1ccccc1Cl] | 534.2 |
| 47 | 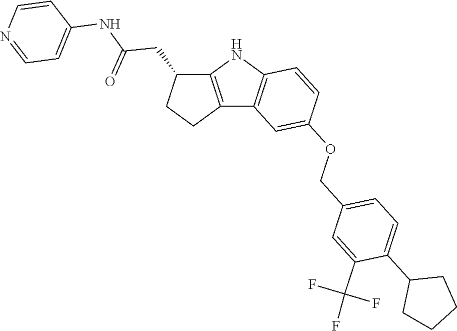 [SMILES: O=C(C[C@H]1CCc2c1[nH]c1ccc(OCc3ccc(C4CCCC4)c(C(F)(F)F)c3)cc21)Nc1ccncc1] | 534.3 |
| 48 | 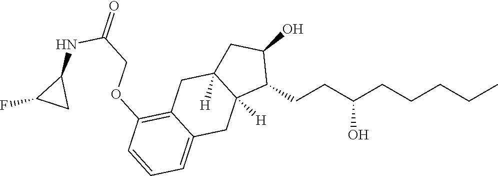 | 470.3 (M + Na) |
| 49 | 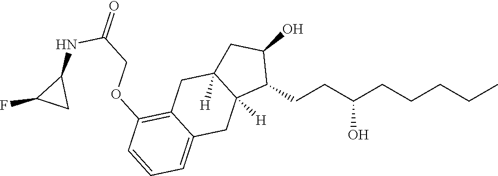 | 470.2 (M + Na) |
| 50 | 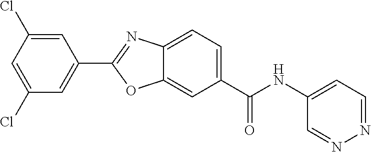 [SMILES: O=C(Nc1ccnnc1)c1ccc2nc(-c3cc(Cl)cc(Cl)c3)oc2c1] | 383.0 (M − H) |
| 51 | 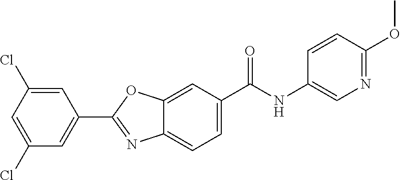 [SMILES: COc1ccc(NC(=O)c2ccc3nc(-c4cc(Cl)cc(Cl)c4)oc3c2)cn1] | 414.0 |
| 52 | 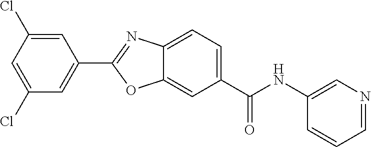 [SMILES: O=C(Nc1cccnc1)c1ccc2nc(-c3cc(Cl)cc(Cl)c3)oc2c1] | 384.0 |
| 53 | 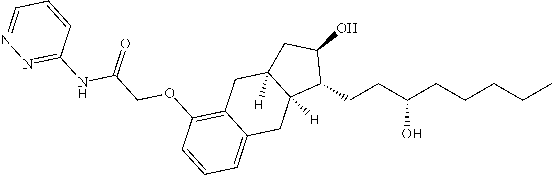 | 468.3 |
| 54 | 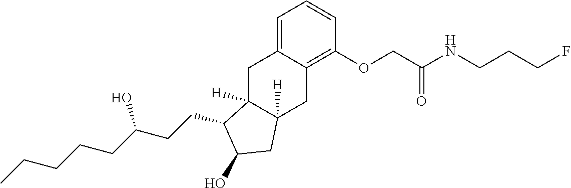 | 472.3 |
| 55 | 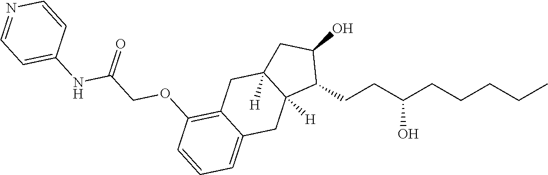 | 467.3 |
| 56 | 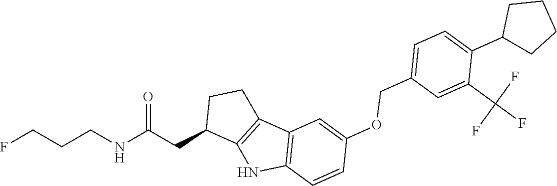 [SMILES: O=C(C[C@H]1CCc2c1[nH]c1ccc(OCc3ccc(C4CCCC4)c(C(F)(F)F)c3)cc21)NCCCF] | 515.3 |
| 57 | 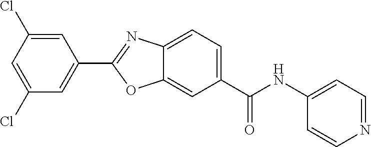 [SMILES: O=C(Nc1ccncc1)c1ccc2nc(-c3cc(Cl)cc(Cl)c3)oc2c1] | 384.0 |

### Example 10: FAAH Substrate Evaluation

Purified recombinant human FAAH (rhFAAH) was purchased from Cayman Chemical (Ann Arbor, MI, USA). The total volume for each incubation was 400 μL containing a final 0.5 ng/L rhFAAH, 1 μM test compound, 1.25% ethanol or 1 μM PF-3845 (FAAH inhibitor), and 0.1% bovine serum albumin in Tris-EDTA buffer at pH 8.0). The positive control was LL-341001. The incubation was conducted at the room temperature. At 0, 5, 15, 30 and 60 minutes, an aliquot of 30 μL reaction mixtures was removed and mixed with 300 μL acetonitrile containing 5 ng/mL terfenadine and 10 ng/mL tolbutamide as internal standards to quench the reaction. The resulting mixture was centrifuged at 4000 rpm, 4° C. for 15 minutes, and 100 μL supernatant was ready for LC-MS/MS analysis to measure the formation of acid metabolite.

### LC-MS/MS Analysis

Acquity Ultra Performance LC system from Waters was used for sample analysis. The chromatography was performed on a reverse phase Kinetex 2.6 μm C18 column, 2.1×30 mm, 100 Å. The mobile phase A comprised of 0.1% formic acid in water and mobile phase B comprised of 0.1% formic acid in acetonitrile with a 2-min run time at the flow rate of 0.8 mL/min for the acid metabolite from positive control or a 1.5 min run time at the flow rate of 0.9 mL/min for the acid metabolite of test compounds. The mass spectrometer (API-5500 and API Q Trap 4000 Applied Biosystems/MDS SCIEX Instruments, Framingham, MA, USA) was operated under ESI positive or negative ion MRM mode.

### Data Analysis

The formation of acid metabolite was monitored and quantified using one calibration point of 1 μM. The observed rate constant (ke) for the acid metabolite formation was calculated by plotting the metabolite concentration versus time of incubation with the slope being ke and is shown in Table 2.

TABLE 2

| Compound | ke | Compound | ke | Compound | ke |
|---|---|---|---|---|---|
| 2 | B | 3 | B | 4 | B |
| 5 | B | 6 | B | 7 | A |
| 8 | NT | 9 | A | 10 | A |
| 11 | A | 12 | C | 13 | D |
| 14 | B | 15 | D | 16 | D |
| 17 | A | 18 | B | 19 | D |
| 20 | D | 21 | D | 22 | B |
| 23 | B | 24 | B | 25 | D |
| 26 | D | 27 | NT | 28 | D |
| 29 | B | 30 | B | 31 | D |
| 32 | C | 33 | D | 34 | D |
| 35 | B | 36 | D | 37 | B |
| 38 | A | 39 | B | 40 | C |
| 41 | B | 42 | B | 43 | B |
| 44 | B | 45 | C | 46 | C |
| 47 | D | 48 | B | 49 | C |
| 50 | A | 51 | A | 52 | A |
| 53 | A | 54 | D | 55 | A |
| 56 | D | 57 | D | | |

A = ke is more than or equal to 1.0; B = ke is less than 1.0 and more than or equal to 0.1; C = ke is less than 0.1 and more than 0; D = ke is 0; NT = not tested.

### Example 11: In Vitro Stability Evaluation in Mouse Plasma

Male CD-1 mouse plasma is purchased from BioIVT (catalog #MSE00PLK2YNN) and thawed in a 37° C. water bath with pH adjusted to 7.4 on Study day. After a pre-warm period of 15 minutes in a 37° C. water bath, 398 μL plasma is spiked with an aliquot of 2 μL stock solution of the test compound or positive control (propantheline) in dimethyl sulfoxide (DMSO) to achieve a final concentration of 1 μM with 0.5% DMSO. After a thorough mix, the mixture is placed back to the 37° C. water bath for incubations. At 0, 15, 30, 60, and 120 minutes, an aliquot of 30 μL reaction mixtures is removed and mixed with 300 μL acetonitrile containing 5 ng/mL terfenadine and 10 ng/mL tolbutamide as internal standards to quench the reaction. The resulting mixture is centrifuged at 4000 rpm, 4° C. for 15 minutes, and 100 μL supernatant is removed and mixed with 100 μL water for liquid chromatography-tandem mass spectrometry (LC-MS/MS) analysis.

### LC-MS/MS Analysis

Shimadzu LC 30-AD HPLC system is used for sample analysis. The chromatography is performed on a reverse phase Kinetex 2.6 μm C18 column, 3.0×30 mm, 100 Å. The mobile phase A comprises of 0.1% formic acid in water and mobile phase B comprises of 0.1% formic acid in acetonitrile with a 2-min run time. The mass spectrometer (API-4000 and API Q Trap 4500 Applied Biosystems/MDS SCIEX Instruments, Framingham, MA, USA) is operated under electrospray ionization (ESI) positive or negative ion multiple reaction monitoring (MRM) mode.

### Data Analysis

Percent compound remaining at a specific time point is calculated based on the peak area ratios at time 0 (as 100%). The observed rate constant (k_{obs}) for the metabolism of test compounds is calculated by plotting the natural log of percentage compound remaining versus time of incubation with the slope being k_{obs}. The half-life (t₁/2) is determined according to the following equation: t_{1/2}=0.693/k_{obs}.

### Example 12: In Vivo Tissue Distribution Studies in Male CD-1 Mice

Male CD-1 mice (n=6 per group), 7-10 weeks old, are acclimated to the study room for a minimum of 3 days before dose administration in the studies. The test compounds are formulated in 1% N-methyl-2-pyrrolidone (NMP) and 1% Solutol in phosphate buffered saline (PBS) at 0.1 mg/mL clear solution and the dose volume was 10 mL/kg. The peripherally restricted FAAH inhibitor LL-650021 is formulated in 0.5% carboxymethyl cellulose in water at 0.1 mg/mL and the dose volume is 10 mL/kg. The concentrations of the formulation are determined to meet the acceptance criteria of within 20% of the target values.

The test compounds are administered to non-fasted mice at 1 mg/kg via subcutaneous (SC) injection or oral gavage (PO) with or without pretreatment of 1 mg/kg LL-650021 1 hour prior to test compound administration. At 1, 4, and 8 hours post-dose, the animals (n=2 per time point) are euthanized using CO₂ inhalation. A blood sample (0.3 mL) is collected from saphenous vein or other suitable site into pre-chilled K₂EDTA tube and placed on wet ice and brain and liver are harvested. The blood samples are centrifuged at 3200 g, 4° C. for 10 minutes and the plasma samples are transferred into polypropylene tubes, quick frozen over dry ice and kept at −60° C. or lower until analysis. The tissues are washed with cold saline, wiped dry, weighed, and then homogenized in 15 mM PBS (pH 7.4):methanol=2:1 buffer at the ratio of 1:10 (1 g tissue with 10 mL buffer resulting in 11-fold dilution). The tissue homogenates are kept at −60° C. or lower until analysis.

### Sample Extraction

The plasma and tissue homogenates are extracted by protein precipitation. An aliquot of 10-50 μL plasma or 40-50 μL tissue homogenates is protein precipitated by adding 200-800 μL acetonitrile containing internal standards (10 ng/mL LL-120001 and 100 ng/mL of celecoxib, dexamethasone, glyburide, labetalol, tolbutamide, and verapamil), vortex-mixed for 10 min at 800 rpm and centrifuged at 4000 rpm, 4° C. for 15 minutes. The supernatant is transferred to the 96-well plate and centrifuged at 4000 rpm, 4° C. for 5 minutes before injected for LC-MS/MS analysis, or 200 μL supernatant is transferred to the 96-well plate, evaporated to dryness under a stream of nitrogen at 25° C., reconstituted with 50 μL of 70% acetonitrile, vortex-mixed for 10 min at 800 rpm and centrifuged at 4000 rpm, 4° C. for 5 minutes before injected for LC-MS/MS analysis.

### LC-MS/MS Analysis

Acquity Ultra Performance LC system from Waters is used for sample analysis. The separations are performed on a ACQUITY UPLC BEH C18 column (50×2.10 mm; 1.7 m) at 50° C. with a flow rate of 0.6 mL/min. Mobile phase A consists of 2 mM ammonium acetate in methanol:water 5:95 and mobile phase B consists of 2 mM ammonium acetate in acetonitrile:water 95:5. Chromatography uses a linear gradient starting at 2% mobile phase B, 2% to 90% mobile phase B over 2.6 minutes, maintained at 90% B wash for 0.2 minutes, and a re-equilibration at 2% B for 0.2 minutes. An aliquot of 2-9 μL sample is injected. The mass spectrometer (API-6500+, Applied Biosystems/MDS SCIEX Instruments, Framingham, MA, USA) is operated under ESI in positive ion or negative ion MRM mode.

What is claimed is:

1. A pharmaceutical composition comprising a fatty acid amide hydrolase (FAAH) cleavable prodrug of Formula (I), or a pharmaceutically acceptable salt or solvate thereof, 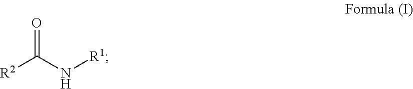
wherein:
R¹ is an amide prodrug moiety, wherein the prodrug of Formula (I) is enzymatically cleaved by fatty acid amide hydrolase (FAAH) in the brain to release 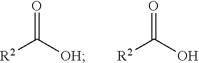
is a moiety that modulates a target in the brain; and a pharmaceutically acceptable excipient; further comprising a peripherally restricted FAAH inhibitor.

2. The pharmaceutical composition of claim 1, or a pharmaceutically acceptable salt or solvate thereof, wherein the target is selected from sphingosine-1-phosphate receptor 1 (S1P1), lysophosphatidic acid receptor 1 (LPA1), G-protein coupled receptor 120 (GPR120), prostacyclin (PGI2), and transthyretin (TTR).

3. The pharmaceutical composition of claim 2, or a pharmaceutically acceptable salt or solvate thereof, wherein the target is S1P1.

4. The pharmaceutical composition of claim 3, or a pharmaceutically acceptable salt or solvate thereof, wherein R² is selected from

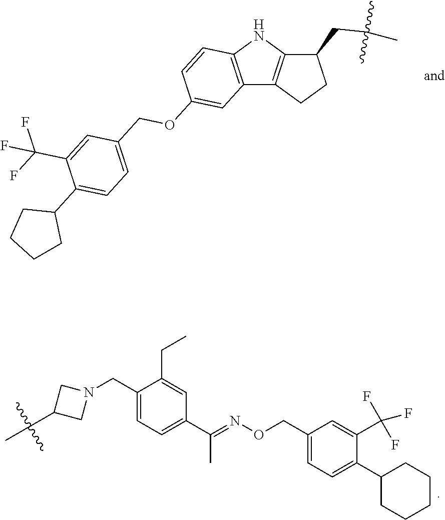 [SMILES: *C[C@H]1CCc2c1[nH]c1ccc(OCc3ccc(C4CCCC4)c(C(F)(F)F)c3)cc21 ; *C1CN(Cc2ccc(/C(C)=N/OCc3ccc(C4CCCCC4)c(C(F)(F)F)c3)cc2CC)C1]

5. The pharmaceutical composition of claim 2, or a pharmaceutically acceptable salt or solvate thereof, wherein the target is LPA1.

6. The pharmaceutical composition of claim 5, or a pharmaceutically acceptable salt or solvate thereof, wherein R² is selected from

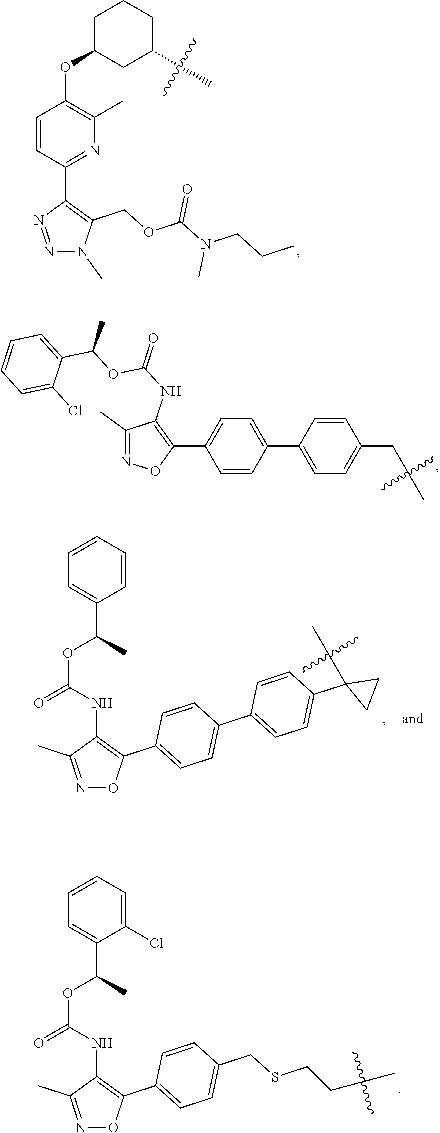

7. The pharmaceutical composition of claim 2, or a pharmaceutically acceptable salt or solvate thereof, wherein the target is GPR120.

8. The pharmaceutical composition of claim 7, or a pharmaceutically acceptable salt or solvate thereof, wherein R² is

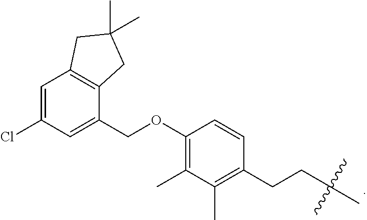 [SMILES: *CCc1ccc(OCc2cc(Cl)cc3c2CC(C)(C)C3)c(C)c1C]

9. The pharmaceutical composition of claim 2, or a pharmaceutically acceptable salt or solvate thereof, wherein the target is TTR.

10. The pharmaceutical composition of claim 9, or a pharmaceutically acceptable salt or solvate thereof, wherein R² is selected from

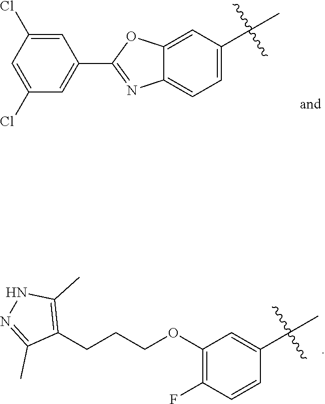 [SMILES: *c1ccc2nc(-c3cc(Cl)cc(Cl)c3)oc2c1 ; *c1ccc(F)c(OCCCc2c(C)n[nH]c2C)c1]

11. The pharmaceutical composition of claim 2, or a pharmaceutically acceptable salt or solvate thereof, wherein the target is PGI2.

12. The pharmaceutical composition of claim 11, or a pharmaceutically acceptable salt or solvate thereof, wherein R² is

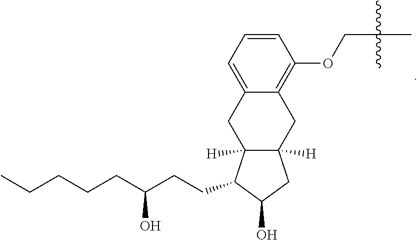 [SMILES: *COc1cccc2c1C[C@H]1C[C@@H](O)[C@H](CC[C@@H](O)CCCCC)[C@H]1C2]

13. The pharmaceutical composition of any one of claims 1-12, or a pharmaceutically acceptable salt or solvate thereof, wherein R¹ is selected from optionally substituted C₁₋₆alkyl, optionally substituted C₃₋₆cycloalkyl, optionally substituted C₂₋₉heterocycloalkyl, optionally substituted C₆₋₁₀aryl, and optionally substituted C₁₋₉heteroaryl.

14. The pharmaceutical composition of any one of claims 1-13, or a pharmaceutically acceptable salt or solvate thereof, wherein R¹ is selected from optionally substituted C₁₋₆alkyl, optionally substituted C₃₋₆cycloalkyl, optionally substituted C₂₋₉heterocycloalkyl, optionally substituted C₆₋₁₀aryl, and optionally substituted C₁₋₉heteroaryl, wherein C₁₋₆alkyl, C₃₋₆cycloalkyl, C₂₋₉heterocycloalkyl, C₆₋₁₀aryl, C₁₋₉heteroaryl are optionally substituted with 1, 2, 3, 4, or 5 groups independently selected from halogen, —CN, —OH, —NH₂, —N(H)(C₁₋₆alkyl), N(C₁₋₆alkyl)₂, —C(O)OH, —C(O)O—C₁₋₆alkyl, —C(O)NH₂, —C(O)N(H)(C₁₋₆alkyl), —C(O)N(C₁₋₆alkyl)₂, —C(O)C₁₋₆alkyl, —S(O)₂C₁₋₆alkyl, —S(O)₂NH₂, —S(O)₂N(H)(C₁₋₆alkyl), —S(O)₂N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl.

15. The pharmaceutical composition of any one of claims 1-14, or a pharmaceutically acceptable salt or solvate thereof, wherein R¹ is selected from unsubstituted C₁₋₆alkyl, unsubstituted C₃₋₆ cycloalkyl, unsubstituted C₂₋₉heterocycloalkyl, unsubstituted C₆₋₁₀aryl, and unsubstituted C₁₋₉heteroaryl.

16. The pharmaceutical composition of any one of claims 1-14, or a pharmaceutically acceptable salt or solvate thereof, wherein the R¹ is selected from

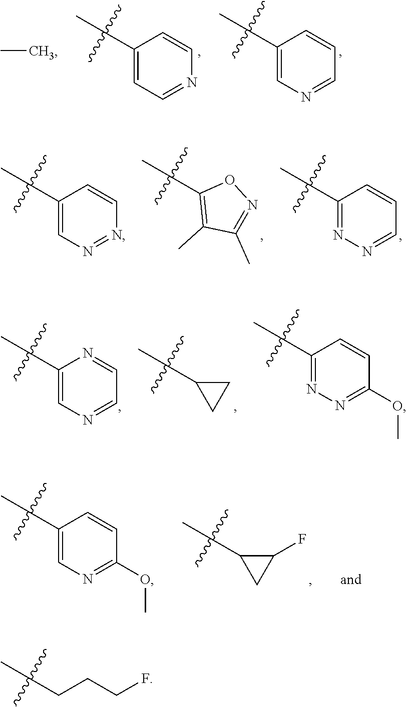

17. The pharmaceutical composition of claim 1 or claim 2, or a pharmaceutically acceptable salt or solvate thereof, wherein the fatty acid amide hydrolase (FAAH) cleavable prodrug of Formula (I) is selected from:

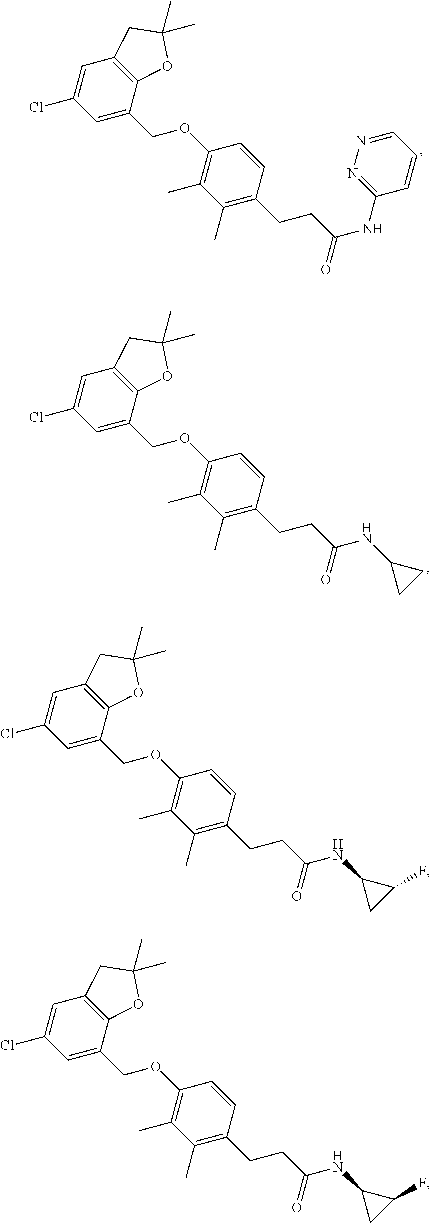 [SMILES: Cc1c(CCC(=O)Nc2cccnn2)ccc(OCc2cc(Cl)cc3c2OC(C)(C)C3)c1C ; Cc1c(CCC(=O)NC2CC2)ccc(OCc2cc(Cl)cc3c2OC(C)(C)C3)c1C ; Cc1c(CCC(=O)N[C@@H]2C[C@H]2F)ccc(OCc2cc(Cl)cc3c2OC(C)(C)C3)c1C ; Cc1c(CCC(=O)N[C@@H]2C[C@@H]2F)ccc(OCc2cc(Cl)cc3c2OC(C)(C)C3)c1C]

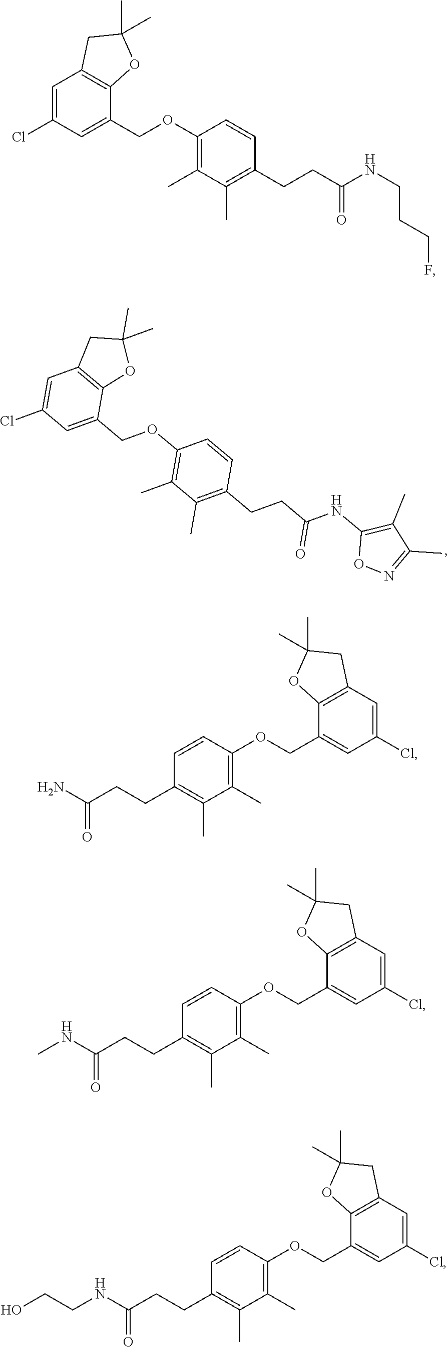

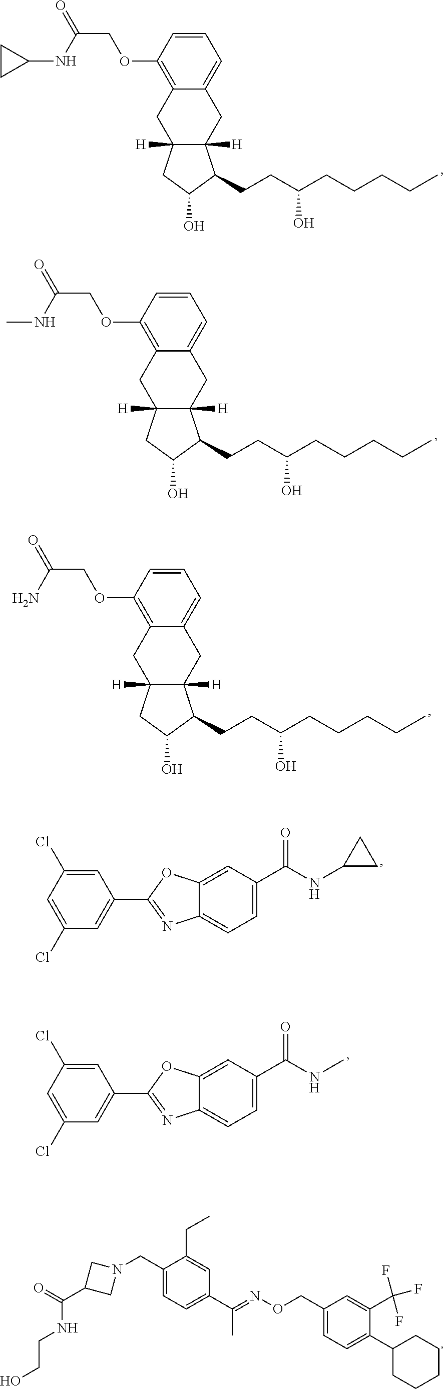

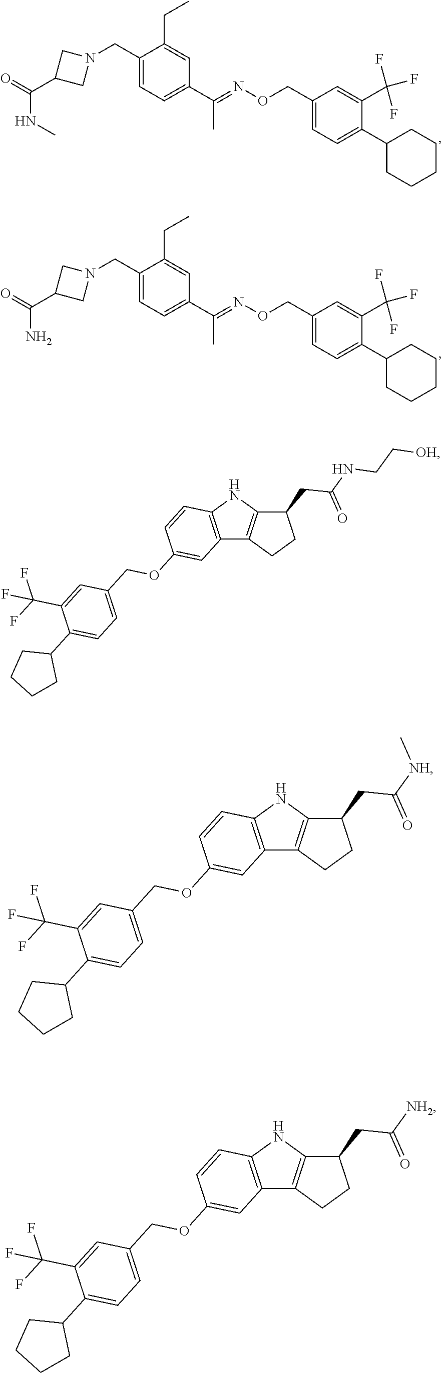 [SMILES: CCc1cc(/C(C)=N/OCc2ccc(C3CCCCC3)c(C(F)(F)F)c2)ccc1CN1CC(C(=O)NC)C1 ; CCc1cc(/C(C)=N/OCc2ccc(C3CCCCC3)c(C(F)(F)F)c2)ccc1CN1CC(C(N)=O)C1 ; O=C(C[C@H]1CCc2c1[nH]c1ccc(OCc3ccc(C4CCCC4)c(C(F)(F)F)c3)cc21)NCCO ; CNC(=O)C[C@H]1CCc2c1[nH]c1ccc(OCc3ccc(C4CCCC4)c(C(F)(F)F)c3)cc21 ; NC(=O)C[C@H]1CCc2c1[nH]c1ccc(OCc3ccc(C4CCCC4)c(C(F)(F)F)c3)cc21]

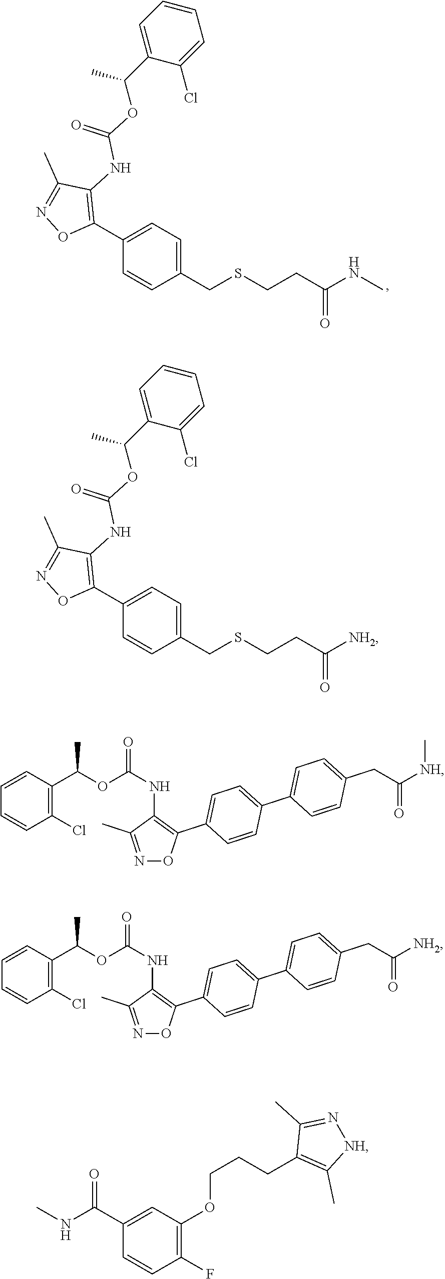

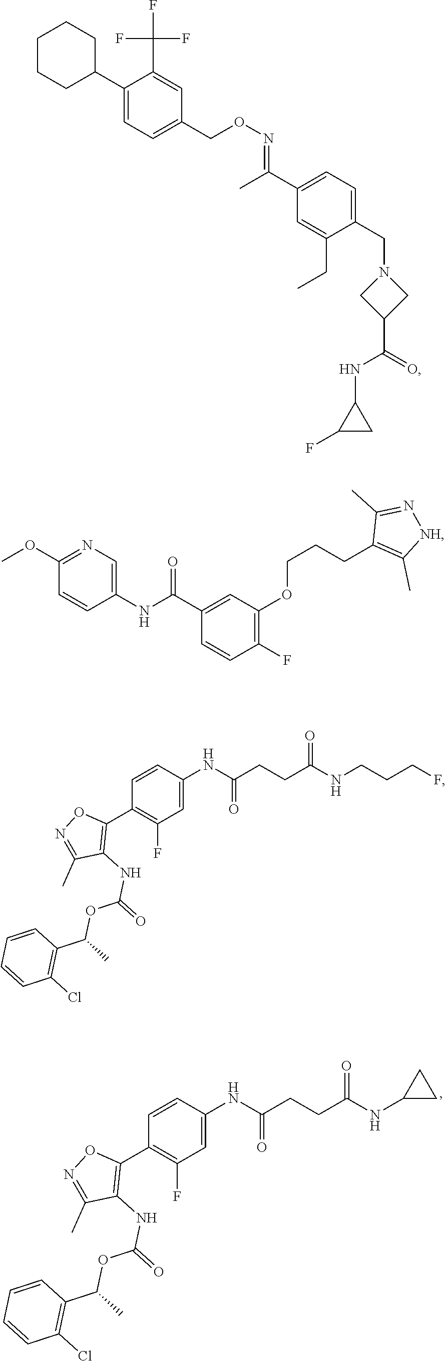 [SMILES: CCc1cc(/C(C)=N/OCc2ccc(C3CCCCC3)c(C(F)(F)F)c2)ccc1CN1CC(C(=O)NC2CC2F)C1 ; COc1ccc(NC(=O)c2ccc(F)c(OCCCc3c(C)n[nH]c3C)c2)cn1 ; Cc1noc(-c2ccc(NC(=O)CCC(=O)NCCCF)cc2F)c1NC(=O)O[C@H](C)c1ccccc1Cl ; Cc1noc(-c2ccc(NC(=O)CCC(=O)NC3CC3)cc2F)c1NC(=O)O[C@H](C)c1ccccc1Cl]

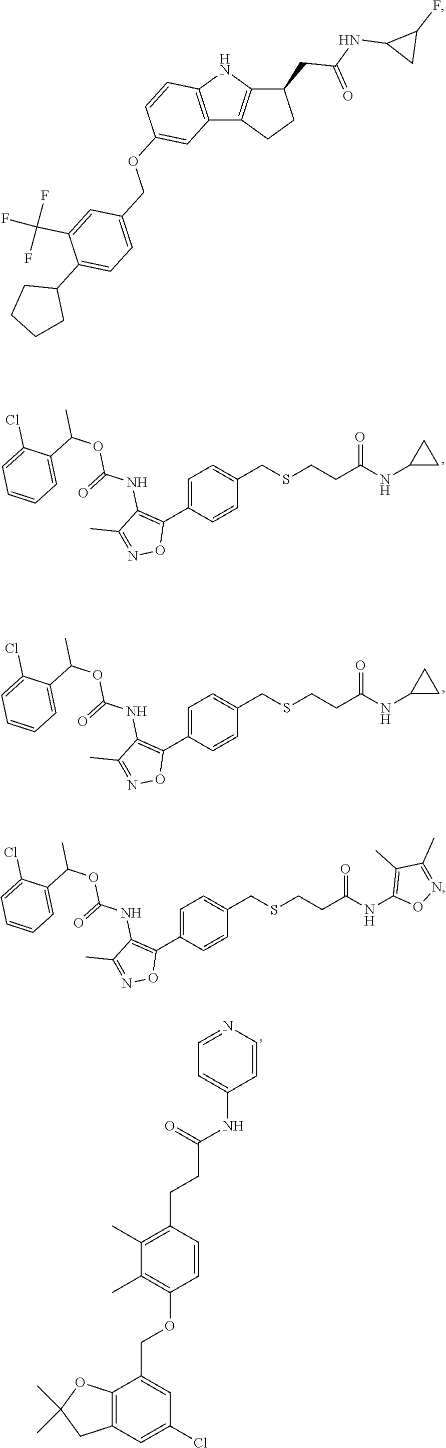 [SMILES: O=C(C[C@H]1CCc2c1[nH]c1ccc(OCc3ccc(C4CCCC4)c(C(F)(F)F)c3)cc21)NC1CC1F ; Cc1noc(-c2ccc(CSCCC(=O)NC3CC3)cc2)c1NC(=O)OC(C)c1ccccc1Cl ; Cc1noc(-c2ccc(CSCCC(=O)NC3CC3)cc2)c1NC(=O)OC(C)c1ccccc1Cl ; Cc1noc(NC(=O)CCSCc2ccc(-c3onc(C)c3NC(=O)OC(C)c3ccccc3Cl)cc2)c1C ; Cc1c(CCC(=O)Nc2ccncc2)ccc(OCc2cc(Cl)cc3c2OC(C)(C)C3)c1C]

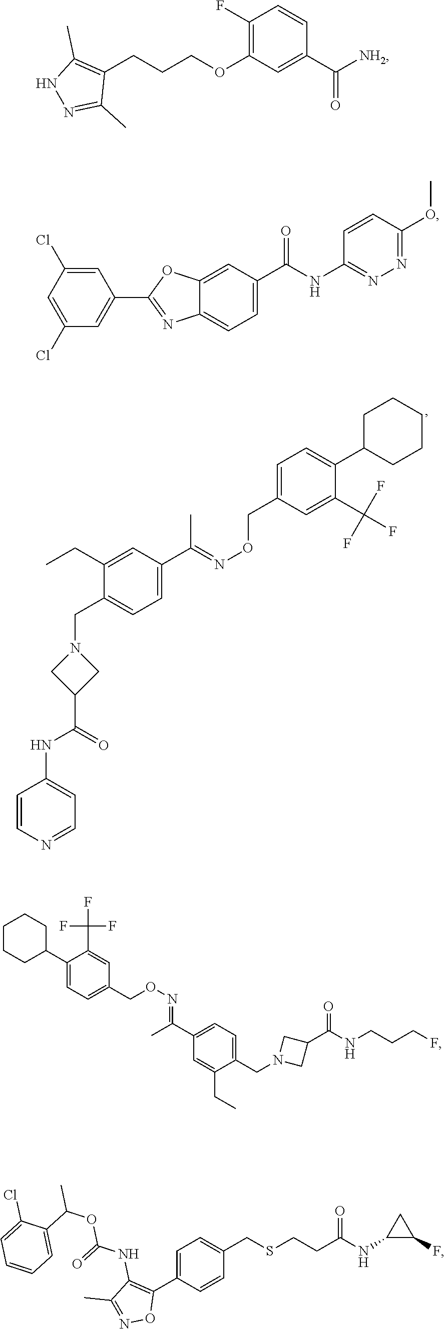 [SMILES: Cc1n[nH]c(C)c1CCCOc1cc(C(N)=O)ccc1F ; COc1ccc(NC(=O)c2ccc3nc(-c4cc(Cl)cc(Cl)c4)oc3c2)nn1 ; CCc1cc(/C(C)=N/OCc2ccc(C3CCCCC3)c(C(F)(F)F)c2)ccc1CN1CC(C(=O)Nc2ccncc2)C1 ; CCc1cc(/C(C)=N/OCc2ccc(C3CCCCC3)c(C(F)(F)F)c2)ccc1CN1CC(C(=O)NCCCF)C1 ; Cc1noc(-c2ccc(CSCCC(=O)N[C@@H]3C[C@H]3F)cc2)c1NC(=O)OC(C)c1ccccc1Cl]

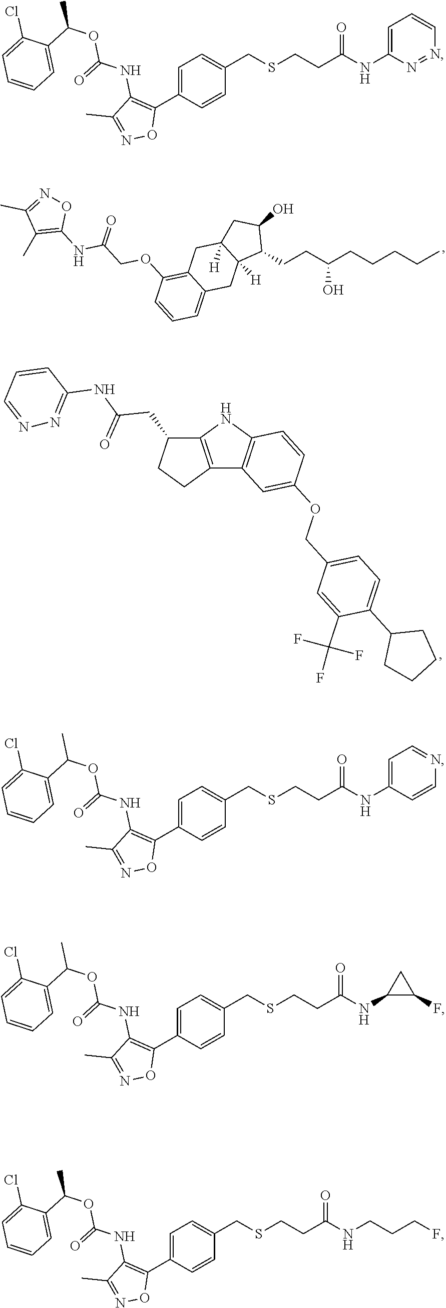

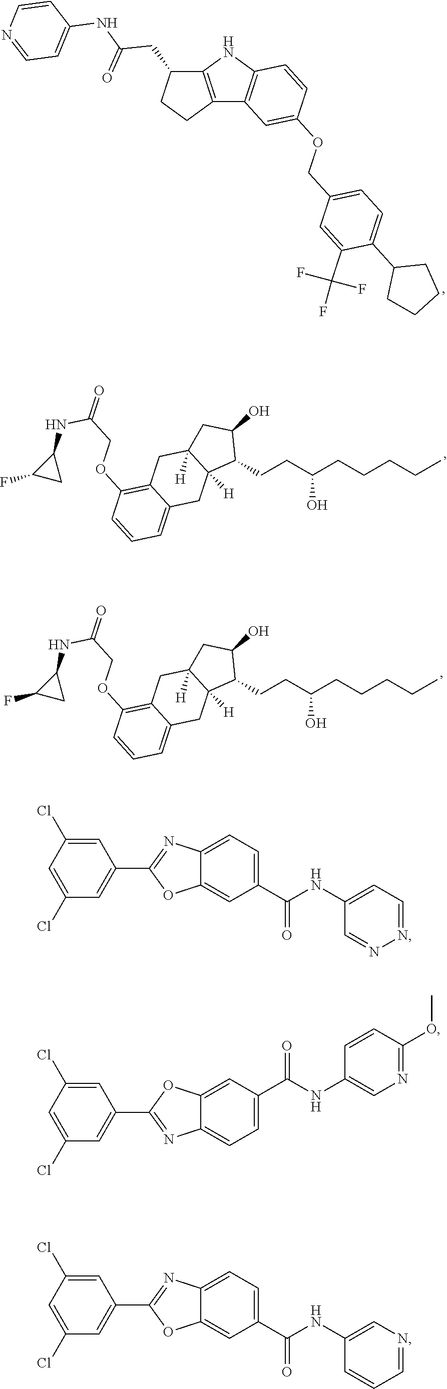

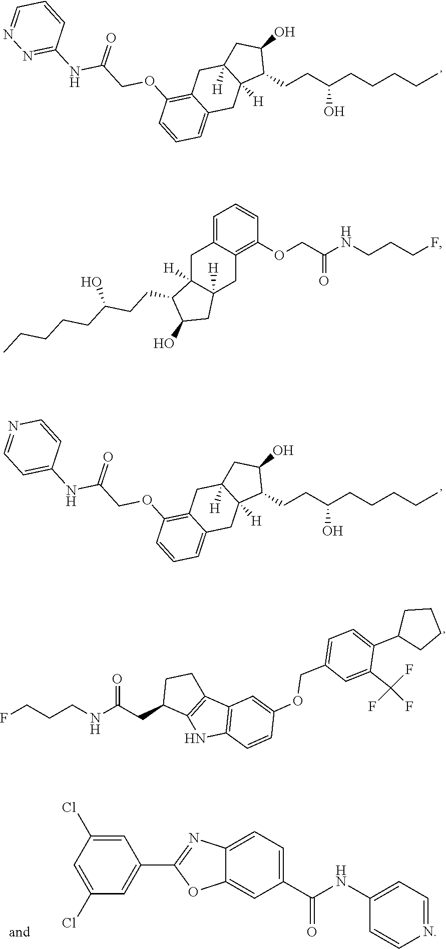

18. The pharmaceutical composition of any one of claims 1-17, or a pharmaceutically acceptable salt or solvate thereof, wherein the peripherally restricted FAAH inhibitor is ASP-3652.

19. A method of treating a CNS disease or disorder in a patient in need thereof comprising administering to the patient a therapeutically effective amount of a pharmaceutical composition of any one of claims 1-18, or a pharmaceutically acceptable salt or solvate thereof.

20. The method of claim 19, wherein the CNS disease or disorder is selected from multiple sclerosis, amyotrophic lateral sclerosis, Huntington's disease, Parkinson's disease, and Alzheimer's disease.

21. The method of claim 19, wherein the CNS disease or disorder is selected from epilepsy, ischemic stroke, traumatic brain injury, and autoimmune encephalomyelitis.

22. The method of claim 19, wherein the CNS disease or disorder is selected from ischemic stroke, schizophrenia, depression, mood disorders, attention deficit hyperactivity disorder, post-traumatic stress disorder, and Alzheimer-type dementia.

23. The method of claim 19, wherein the CNS disease or disorder is selected from familial amyloidotic polyneuropathy, familial leptomeningeal amyloidosis, Alzheimer's disease, stroke, dementia, transitory focal neurological episodes, cognitive dysfunction, and CNS amyloidosis.

24. The method of claim 19, wherein the CNS disease or disorder is selected from Degos disease, reversible cerebral vasoconstriction syndrome, Sneddon's syndrome, amyloid-beta-related angiopathy, Susac syndrome, and neurosarcoidosis.

25. A method of increasing the 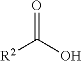
concentration in the brain of a patient comprising administering to the patient a pharmaceutical composition of any one of claims 1-18, or a pharmaceutically acceptable salt or solvate thereof; wherein the ratio of brain to periphery 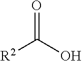
concentration is increased to greater than 1:1.

26. The method of claim 25, wherein the ratio of brain to periphery 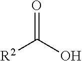
concentration is increased to greater than 2:1.

27. A compound, or a pharmaceutically acceptable salt or solvate thereof, of Formula (II): 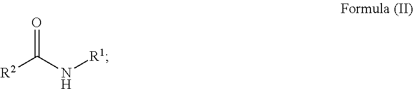
wherein:
R¹ is an amide prodrug moiety, wherein the amide prodrug moiety is enzymatically cleaved by fatty acid amide hydrolase (FAAH) in the brain to release 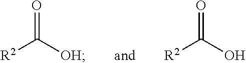
is a moiety that modulates S1P1 in the brain.

28. The compound of claim 27, or a pharmaceutically acceptable salt or solvate thereof, wherein
R² is selected from

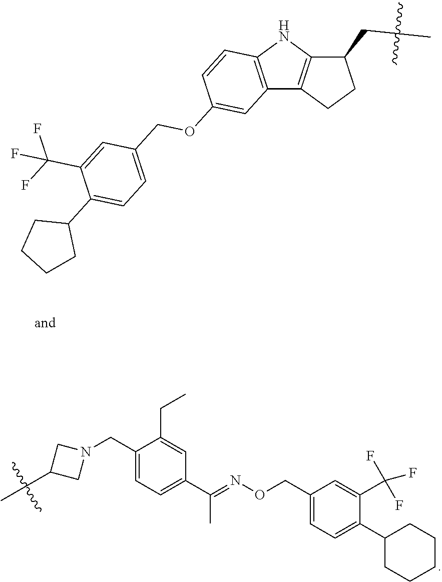 [SMILES: *C[C@H]1CCc2c1[nH]c1ccc(OCc3ccc(C4CCCC4)c(C(F)(F)F)c3)cc21 ; *C1CN(Cc2ccc(/C(C)=N/OCc3ccc(C4CCCCC4)c(C(F)(F)F)c3)cc2CC)C1]

29. A compound, or a pharmaceutically acceptable salt or solvate thereof, of Formula (III): 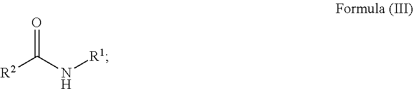
wherein:
R¹ is an amide prodrug moiety, wherein the amide prodrug moiety is enzymatically cleaved by fatty acid amide hydrolase (FAAH) in the brain to release 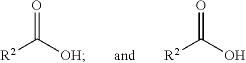
is a moiety that modulates LPA1 in the brain.

30. The compound of claim 29, or a pharmaceutically acceptable salt or solvate thereof, wherein
R² is selected from

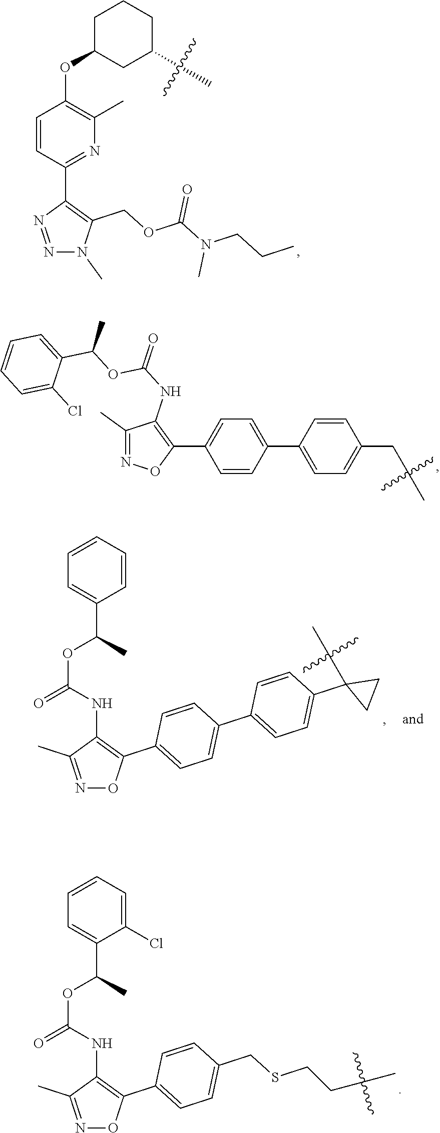

31. A compound, or a pharmaceutically acceptable salt or solvate thereof, of Formula (IV): 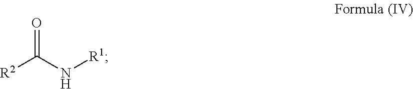
wherein:
R¹ is an amide prodrug moiety, wherein the amide prodrug moiety is enzymatically cleaved by fatty acid amide hydrolase (FAAH) in the brain to release 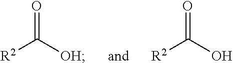
is a moiety that modulates GPR120 in the brain.

32. The compound of claim 31, or a pharmaceutically acceptable salt or solvate thereof, wherein
R² is

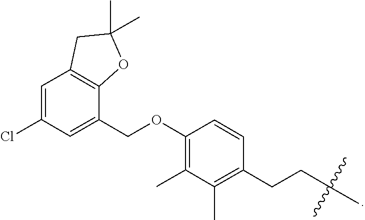 [SMILES: *CCc1ccc(OCc2cc(Cl)cc3c2OC(C)(C)C3)c(C)c1C]

33. The compound of any one of claims 27-32, or a pharmaceutically acceptable salt or solvate thereof, wherein R¹ is selected from optionally substituted C₁₋₆alkyl, optionally substituted C₃₋₆cycloalkyl, optionally substituted C₂₋₉heterocycloalkyl, optionally substituted C₆₋₁₀aryl, and optionally substituted C₁₋₉heteroaryl.

34. The compound of any one of claims 27-33, or a pharmaceutically acceptable salt or solvate thereof, wherein R¹ is selected from C₁₋₆alkyl, C₃₋₆cycloalkyl, C₂₋₉heterocycloalkyl, C₆₋₁₀ aryl, and C₁₋₉heteroaryl, wherein C₁₋₆alkyl, C₃₋₆cycloalkyl, C₂₋₉heterocycloalkyl, C₆₋₁₀aryl, and C₁₋₉heteroaryl are optionally substituted with 1, 2, 3, 4, or 5 groups independently selected from halogen, —CN, —OH, —NH₂, —N(H)(C₁₋₆alkyl), N(C₁₋₆alkyl)₂, —C(O)OH, —C(O)O—C₁₋₆alkyl, —C(O)NH₂, —C(O)N(H)(C₁₋₆alkyl), —C(O)N(C₁₋₆alkyl)₂, —C(O)C₁₋₆alkyl, —S(O)₂C₁₋₆alkyl, —S(O)₂NH₂, —S(O)₂N(H)(C₁₋₆alkyl), —S(O)₂N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl.

35. The compound of any one of claims 27-34, or a pharmaceutically acceptable salt or solvate thereof, wherein R¹ is selected from unsubstituted C₁₋₆alkyl, unsubstituted C₃₋₆cycloalkyl, unsubstituted C₂₋₉heterocycloalkyl, unsubstituted C₆₋₁₀aryl, and unsubstituted C₁₋₉heteroaryl.

36. A compound, or a pharmaceutically acceptable salt or solvate thereof, of Formula (V): 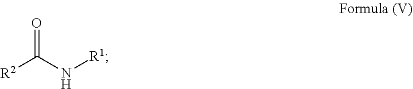
wherein:
R¹ is an amide prodrug moiety, wherein the amide prodrug moiety is enzymatically cleaved by fatty acid amide hydrolase (FAAH) in the brain to release 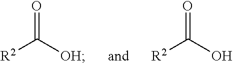
is a moiety that modulates TTR in the brain.

37. The compound of claim 36, or a pharmaceutically acceptable salt or solvate thereof, wherein
R² is

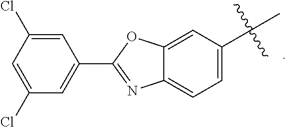 [SMILES: *c1ccc2nc(-c3cc(Cl)cc(Cl)c3)oc2c1]

38. The compound of claim 36 or claim 37, or a pharmaceutically acceptable salt or solvate thereof, wherein R¹ is selected from C₁₋₄alkyl, C₁₋₄haloalkyl, C₃₋₆cycloalkyl, C₆₋₁₀aryl, and C₁₋₉heteroaryl, wherein C₃₋₆cycloalkyl, C₆₋₁₀aryl, and C₁₋₉heteroaryl are optionally substituted with 1, 2, 3, 4, or 5 groups independently selected from halogen, —CN, —OH, —NH₂, —N(H)(C₁₋₆alkyl), N(C₁₋₆alkyl)₂, —C(O)OH, —C(O)O—C₁₋₆alkyl, —C(O)NH₂, —C(O)N(H)(C₁₋₆alkyl), —C(O)N(C₁₋₆alkyl)₂, —C(O)C₁₋₆alkyl, —S(O)₂C₁₋₆alkyl, —S(O)₂NH₂, —S(O)₂N(H)(C₁₋₆alkyl), —S(O)₂N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl.

39. The compound of any one of claims 36-38, or a pharmaceutically acceptable salt or solvate thereof, wherein R¹ is selected from unsubstituted C₁₋₄alkyl, unsubstituted C₃₋₆cycloalkyl, unsubstituted C₆₋₁₀aryl, and unsubstituted C₁₋₉heteroaryl.

40. The compound of claim 36, or a pharmaceutically acceptable salt or solvate thereof, wherein
R² is

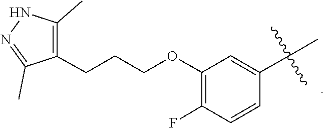 [SMILES: *c1ccc(F)c(OCCCc2c(C)n[nH]c2C)c1]

41. The compound of claim 40, or a pharmaceutically acceptable salt or solvate thereof, wherein R¹ is selected from —CH₃, C₁₋₆haloalkyl, C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with 1, 2, 3, 4, or 5 groups independently selected from halogen, —CN, —OH, —NH₂, —N(H)(C₁₋₆alkyl), N(C₁₋₆alkyl)₂, —C(O)OH, —C(O)O—C₁₋₆alkyl, —C(O)NH₂, —C(O)N(H)(C₁₋₆alkyl), —C(O)N(C₁₋₆alkyl)₂, —C(O)C₁₋₆alkyl, —S(O)₂C₁₋₆alkyl, —S(O)₂NH₂, —S(O)₂N(H)(C₁₋₆alkyl), —S(O)₂N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl.

42. The compound of claim 41, or a pharmaceutically acceptable salt or solvate thereof, wherein R¹ is selected from —CH₃, unsubstituted C₃₋₆cycloalkyl, unsubstituted C₂₋₉heterocycloalkyl, unsubstituted C₆₋₁₀aryl, and unsubstituted C₁₋₉heteroaryl.

43. A compound, or a pharmaceutically acceptable salt or solvate thereof, of Formula (VI): 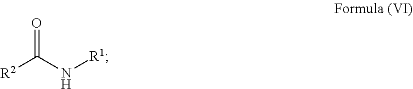
wherein:
R¹ is an amide prodrug moiety, wherein the amide prodrug moiety is enzymatically cleaved by fatty acid amide hydrolase (FAAH) in the brain to release 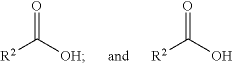
is a moiety that modulates PGI2 in the brain.

44. The compound of claim 43, or a pharmaceutically acceptable salt or solvate thereof, wherein
R² is

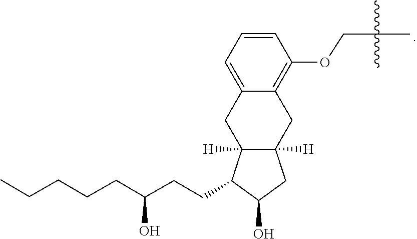 [SMILES: *COc1cccc2c1C[C@H]1C[C@@H](O)[C@H](CC[C@@H](O)CCCCC)[C@H]1C2]

45. The compound of claim 44, or a pharmaceutically acceptable salt or solvate thereof, wherein R¹ is selected from —CH₃, optionally substituted C₃₋₆cycloalkyl, optionally substituted C₂₋₉heterocycloalkyl, optionally substituted C₆₋₁₀aryl, and optionally substituted C₂₋₉heteroaryl, wherein C₃₋₆cycloalkyl, C₂₋₉heterocycloalkyl, C₆₋₁₀aryl, C₁₋₉heteroaryl are optionally substituted with 1, 2, 3, 4, or 5 groups independently selected from halogen, —CN, —OH, —NH₂, —N(H)(C₁₋₆alkyl), N(C₁₋₆alkyl)₂, —C(O)OH, —C(O)O—C₁₋₆alkyl, —C(O)NH₂, —C(O)N(H)(C₁₋₆alkyl), —C(O)N(C₁₋₆alkyl)₂, —C(O)C₁₋₆alkyl, —S(O)₂C₁₋₆alkyl, —S(O)₂NH₂, —S(O)₂N(H)(C₁₋₆alkyl), —S(O)₂N(C₁₋₆alkyl)₂, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl.

46. The compound of claim 45, or a pharmaceutically acceptable salt or solvate thereof, wherein R¹ is selected from —CH₃, unsubstituted C₃₋₆cycloalkyl, unsubstituted C₂₋₉heterocycloalkyl, unsubstituted C₆₋₁₀aryl, and unsubstituted C₁₋₉heteroaryl.

47. The compound of any one of claims 27-46, or a pharmaceutically acceptable salt or solvate thereof, wherein the R¹ is selected from

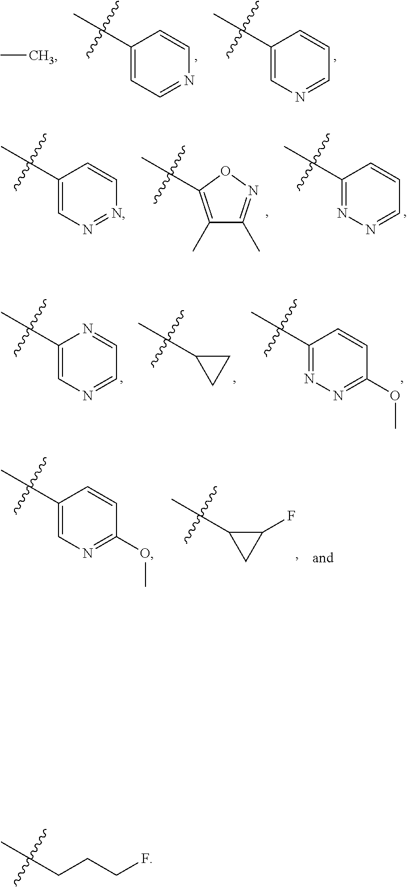

48. The compound of claim 27, or a pharmaceutically acceptable salt or solvate thereof, wherein the compound of Formula (II) is selected from:

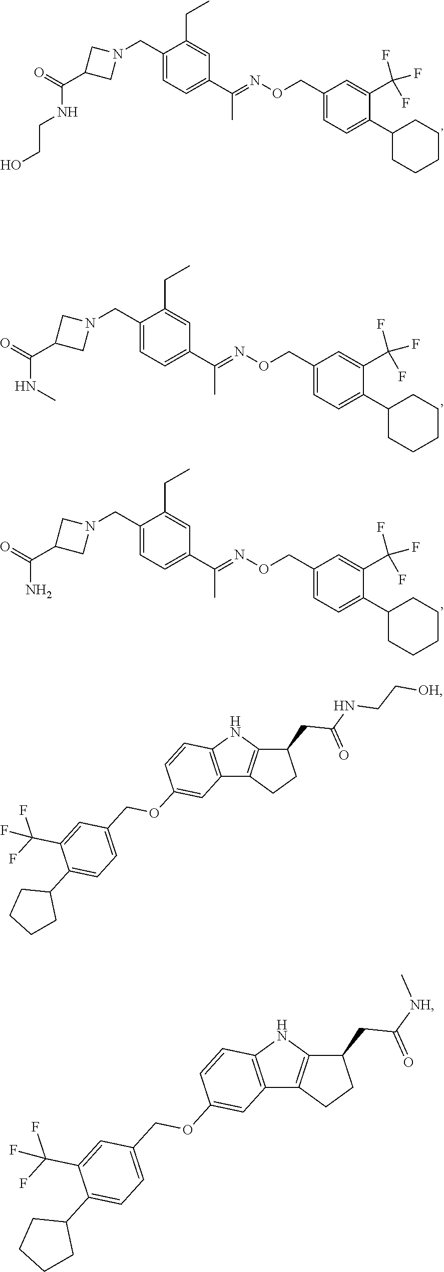

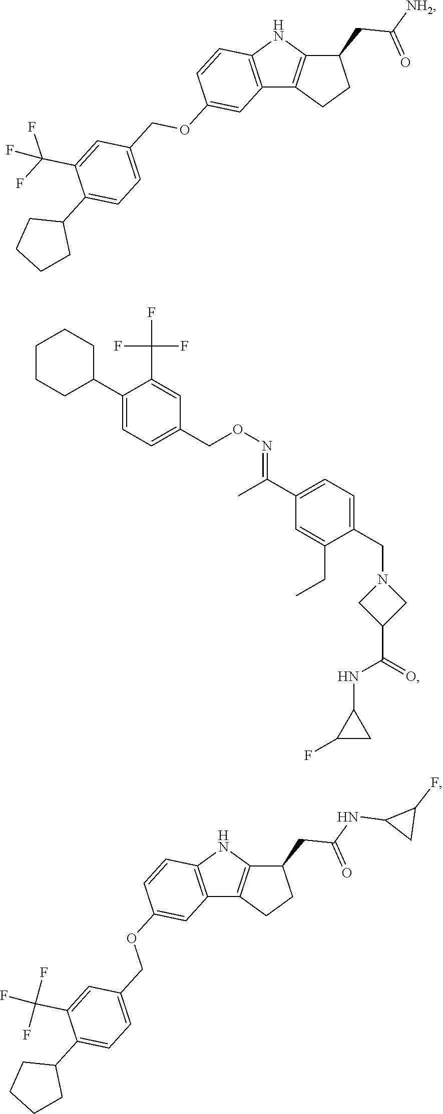 [SMILES: NC(=O)C[C@H]1CCc2c1[nH]c1ccc(OCc3ccc(C4CCCC4)c(C(F)(F)F)c3)cc21 ; CCc1cc(/C(C)=N/OCc2ccc(C3CCCCC3)c(C(F)(F)F)c2)ccc1CN1CC(C(=O)NC2CC2F)C1 ; O=C(C[C@H]1CCc2c1[nH]c1ccc(OCc3ccc(C4CCCC4)c(C(F)(F)F)c3)cc21)NC1CC1F]

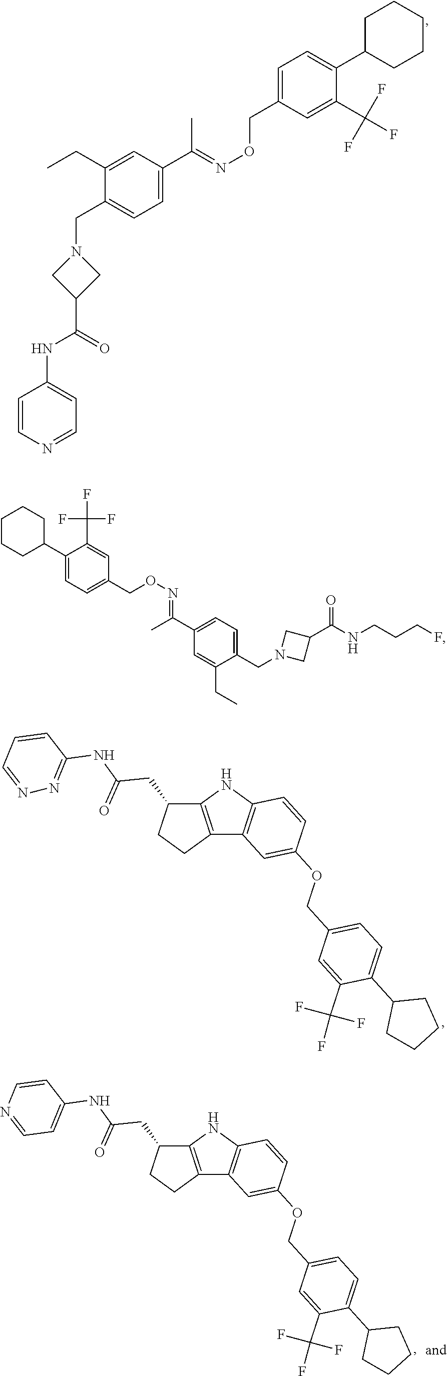 [SMILES: CCc1cc(/C(C)=N/OCc2ccc(C3CCCCC3)c(C(F)(F)F)c2)ccc1CN1CC(C(=O)Nc2ccncc2)C1 ; CCc1cc(/C(C)=N/OCc2ccc(C3CCCCC3)c(C(F)(F)F)c2)ccc1CN1CC(C(=O)NCCCF)C1 ; O=C(C[C@H]1CCc2c1[nH]c1ccc(OCc3ccc(C4CCCC4)c(C(F)(F)F)c3)cc21)Nc1cccnn1 ; O=C(C[C@H]1CCc2c1[nH]c1ccc(OCc3ccc(C4CCCC4)c(C(F)(F)F)c3)cc21)Nc1ccncc1]

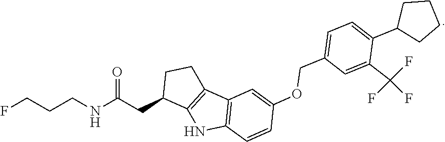 [SMILES: O=C(C[C@H]1CCc2c1[nH]c1ccc(OCc3ccc(C4CCCC4)c(C(F)(F)F)c3)cc21)NCCCF]

49. The compound of claim 29, or a pharmaceutically acceptable salt or solvate thereof, wherein the compound of Formula (III) is selected from:

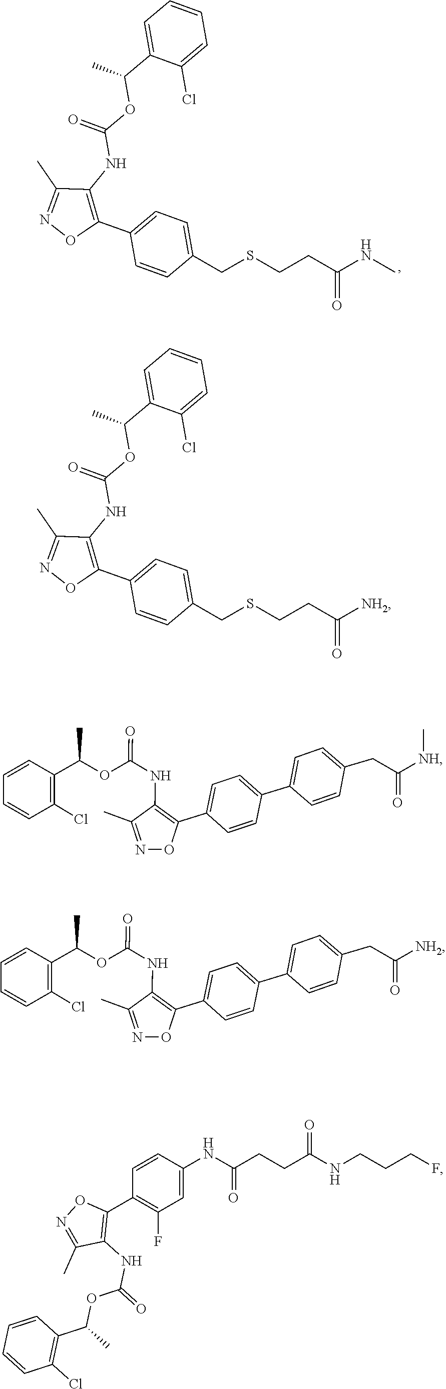

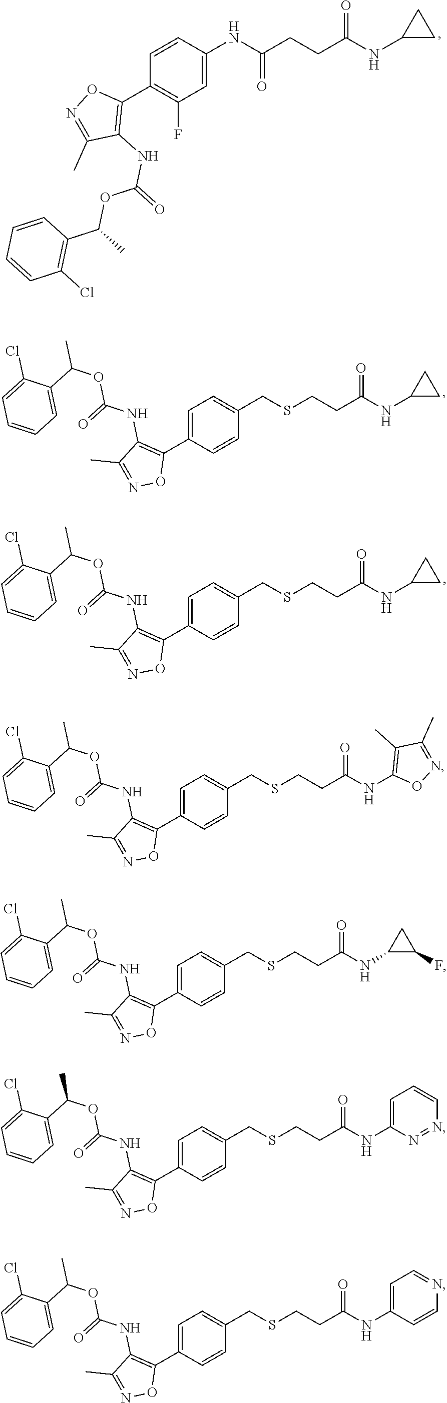

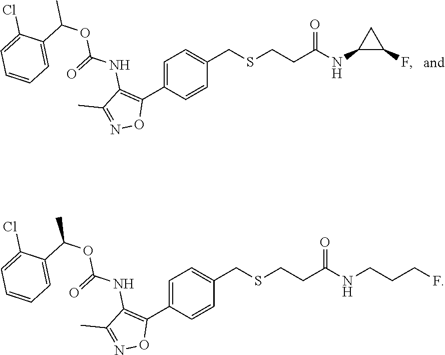 [SMILES: Cc1noc(-c2ccc(CSCCC(=O)N[C@H]3C[C@H]3F)cc2)c1NC(=O)OC(C)c1ccccc1Cl ; Cc1noc(-c2ccc(CSCCC(=O)NCCCF)cc2)c1NC(=O)O[C@H](C)c1ccccc1Cl]

50. The compound of claim 31, or a pharmaceutically acceptable salt or solvate thereof, wherein the compound of Formula (IV) is selected from:

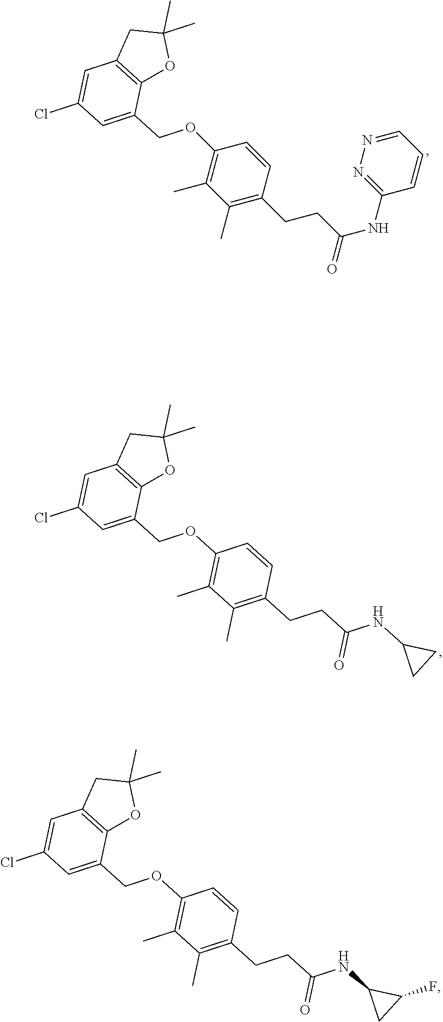 [SMILES: Cc1c(CCC(=O)Nc2cccnn2)ccc(OCc2cc(Cl)cc3c2OC(C)(C)C3)c1C ; Cc1c(CCC(=O)NC2CC2)ccc(OCc2cc(Cl)cc3c2OC(C)(C)C3)c1C ; Cc1c(CCC(=O)N[C@@H]2C[C@H]2F)ccc(OCc2cc(Cl)cc3c2OC(C)(C)C3)c1C]

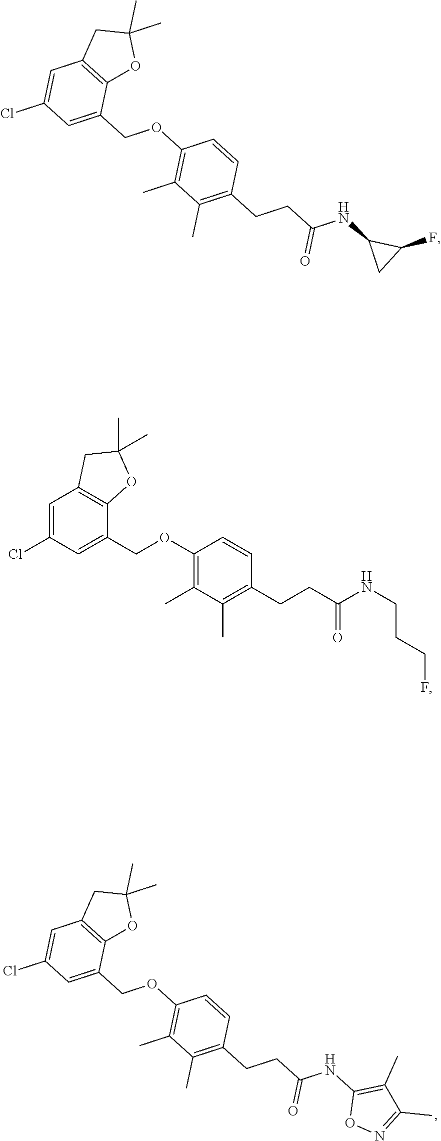 [SMILES: Cc1c(CCC(=O)N[C@@H]2C[C@@H]2F)ccc(OCc2cc(Cl)cc3c2OC(C)(C)C3)c1C ; Cc1c(CCC(=O)NCCCF)ccc(OCc2cc(Cl)cc3c2OC(C)(C)C3)c1C ; Cc1noc(NC(=O)CCc2ccc(OCc3cc(Cl)cc4c3OC(C)(C)C4)c(C)c2C)c1C]

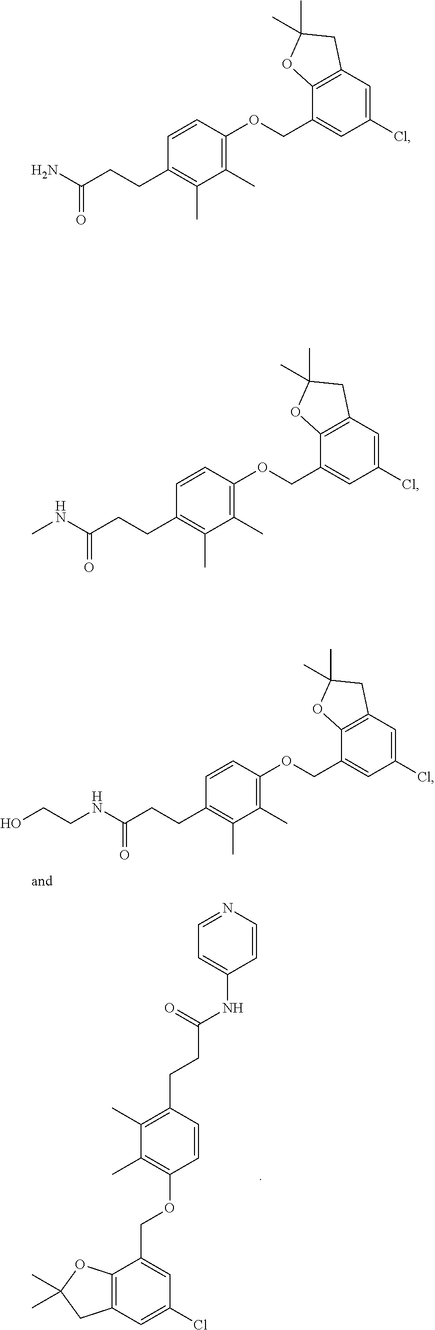 [SMILES: Cc1c(CCC(N)=O)ccc(OCc2cc(Cl)cc3c2OC(C)(C)C3)c1C ; CNC(=O)CCc1ccc(OCc2cc(Cl)cc3c2OC(C)(C)C3)c(C)c1C ; Cc1c(CCC(=O)NCCO)ccc(OCc2cc(Cl)cc3c2OC(C)(C)C3)c1C ; Cc1c(CCC(=O)Nc2ccncc2)ccc(OCc2cc(Cl)cc3c2OC(C)(C)C3)c1C]

51. The compound of claim 36, or a pharmaceutically acceptable salt or solvate thereof, wherein the compound of Formula (V) is selected from:

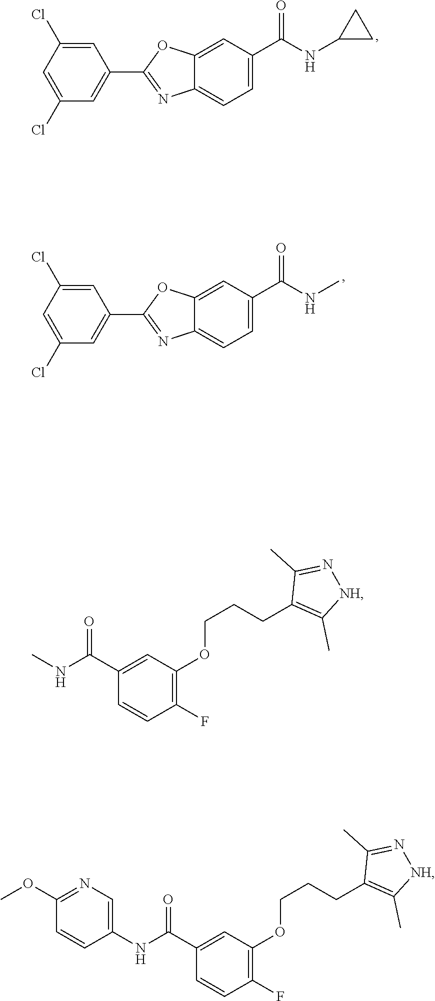

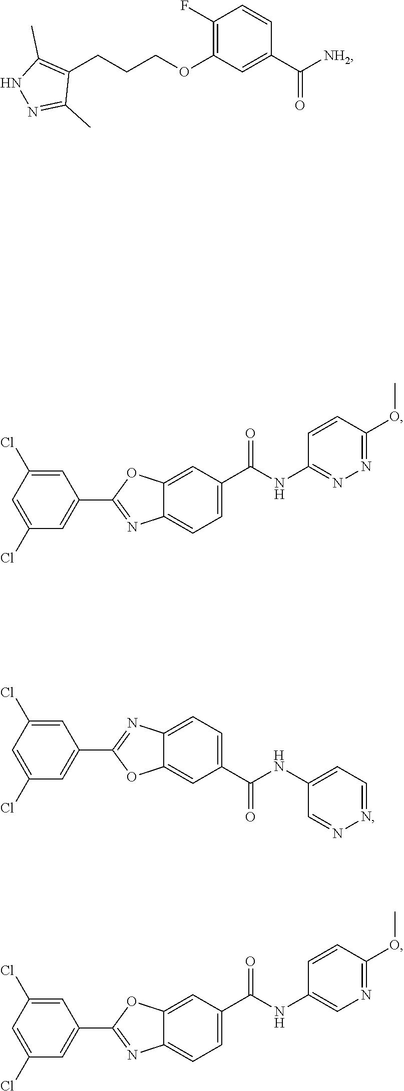 [SMILES: Cc1n[nH]c(C)c1CCCOc1cc(C(N)=O)ccc1F ; COc1ccc(NC(=O)c2ccc3nc(-c4cc(Cl)cc(Cl)c4)oc3c2)nn1 ; O=C(Nc1ccnnc1)c1ccc2nc(-c3cc(Cl)cc(Cl)c3)oc2c1 ; COc1ccc(NC(=O)c2ccc3nc(-c4cc(Cl)cc(Cl)c4)oc3c2)cn1]

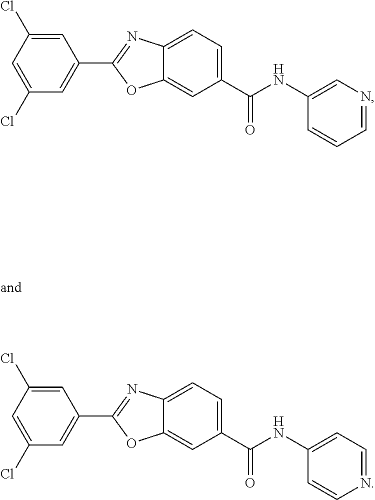 [SMILES: O=C(Nc1cccnc1)c1ccc2nc(-c3cc(Cl)cc(Cl)c3)oc2c1 ; O=C(Nc1ccncc1)c1ccc2nc(-c3cc(Cl)cc(Cl)c3)oc2c1]

52. The compound of claim 43, or a pharmaceutically acceptable salt or solvate thereof, wherein the compound of Formula (VI) is selected from:

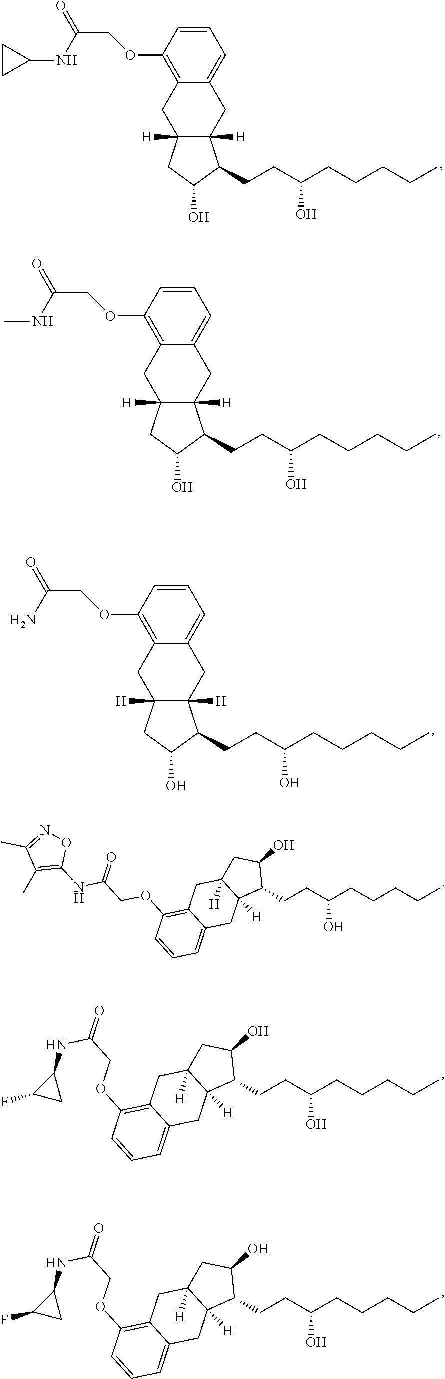

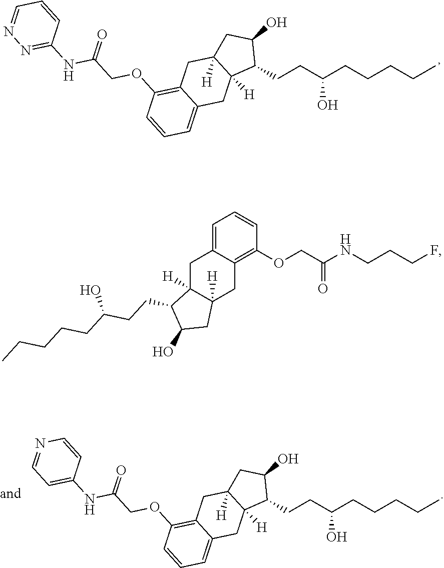

53. A pharmaceutical composition comprising a compound of any one of claims 27-52, or a pharmaceutically acceptable salt or solvate thereof, and a pharmaceutically acceptable excipient.

54. The pharmaceutical composition of claim 53 further comprising a peripherally restricted FAAH inhibitor.

55. The pharmaceutical composition of claim 54 further comprising a peripherally restricted FAAH inhibitor, wherein the peripherally restricted FAAH inhibitor is ASP-3652.

56. A method of treating a CNS disease or disorder in a patient in need thereof comprising administering to the patient a therapeutically effective amount of a pharmaceutical composition of any one of claims 53-55, or a compound of any one of claims 27-52, or a pharmaceutically acceptable salt or solvate thereof.

57. The method of claim 56, wherein the CNS disease or disorder is selected from multiple sclerosis, amyotrophic lateral sclerosis, Huntington's disease, Parkinson's disease, and Alzheimer's disease.

58. The method of claim 56, wherein the CNS disease or disorder is selected from epilepsy, ischemic stroke, traumatic brain injury, and autoimmune encephalomyelitis.

59. The method of claim 56, wherein the CNS disease or disorder is selected from ischemic stroke, schizophrenia, depression, mood disorders, attention deficit hyperactivity disorder, post-traumatic stress disorder, and Alzheimer-type dementia.

60. The method of claim 56, wherein the CNS disease or disorder is selected from familial amyloidotic polyneuropathy, familial leptomeningeal amyloidosis, Alzheimer's disease, stroke, dementia, transitory focal neurological episodes, cognitive dysfunction, and CNS amyloidosis.

61. The method of claim 56, wherein the CNS disease or disorder is selected from Degos disease, reversible cerebral vasoconstriction syndrome, Sneddon's syndrome, amyloid-beta-related angiopathy, Susac syndrome, and neurosarcoidosis.

62. A method of increasing the 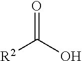
concentration in the brain of a patient comprising administering to the patient a pharmaceutical composition of any one of claims 53-55, or a compound of any one of claims 27-52, or a pharmaceutically acceptable salt or solvate thereof, wherein the ratio of brain to periphery 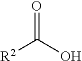
concentration is increased to greater than 1:1.

63. The method of claim 62, the ratio of brain to periphery 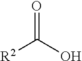
OH concentration is increased to greater than 2:1.
